(19) United States
(12) Patent Application Publication
(10) Pub. No.: US 2004/0137582 A1
(43) Pub. Date: Jul. 15, 2004

(54) Methods for manufacturing polysaccharide derivatives

(76) Inventors: Jonathan S. Dordick, Schenectady, NY (US); Lino Ferreira, Coimbra (PT)

Correspondence Address: ELMORE CRAIG, P.C., 209 MAIN STREET, N. CHELMSFORD, MA, 01863, US

(21) Appl. No.: 10/663,348

(22) Filed: Sep. 15, 2003

Related U.S. Application Data
(60) Provisional application No. 60/410,972, filed on Sep. 16, 2002.
(60) Provisional application No. 60/410,976, filed on Sep. 16, 2002.

(51) Int. Cl. A61K 31/715; C12P 19/04

(52) U.S. Cl. 435/101; 514/54

(57) ABSTRACT

The invention is based upon the discovery that insoluble, polysaccharides, such as inulin and dextran, can be enzymatically modified in an organic solvent. Thus, the invention relates to methods for making a high molecular weight polyhydroxy polymer, such as a polysaccharide, inulin or dextran derivative, comprising reacting an acyl donor and the polymer, such as inulin or dextran, to form an acyl ester of the polymer, such as inulin dextran, in a reaction medium comprising an organic solvent in the presence of a hydrolytic enzyme; methods for making a polymer, such as a polysaccharide, an inulin or dextran polymer, comprising reacting a polymerizable acyl donor and polyhydroxyl polymer in a reaction medium comprising an organic solvent in the presence of a hydrolytic enzyme thereby making an polymeric monomer, such as an inulin monomer, and polymerizing, preferably dimerizing, the monomer, thereby making a novel polymer, such as an inulin polymer. The invention further relates to novel products as can be produced by the processes described herein, pharmaceutical compositions containing them and the use of the novel polymers described herein in methods for the manufacture of a pharmaceutical composition or medicament. Further, the invention relates to a method of delivering an active agent to a patient comprising administering to the patient a pharmaceutical composition described herein.

### RELATED APPLICATIONS

This application claims the benefit of priority of U.S. Serial No. 60/410,972 filed on Sep. 16, 2002 and U.S. Serial No. 60/410,976, filed on Sep. 16, 2002, the contents of which are hereby incorporated by reference.

### BACKGROUND OF THE INVENTION

Hydrogels are three-dimensional, hydrophilic, polymeric networks capable of imbibing large amounts of water or biological fluids. These networks have been used as membranes for separating solutes, wound dressings, drug delivery systems, such as for gene therapy and protein controlled-released systems, and immobilization of enzymes and cells, among others.

In the last few years there have been considerable efforts made to develop hydrogels from sugars and dextran, a bacterial polysaccharide consisting essentially of α-1,6 linked D-glucopyranoside residues with a small percentage of α-1,3 linked side chains. For example, dextran-based hydrogels have been obtained either in a single step using bi-functional crosslinking agents such as isocyanate-type monomers, or in two-steps involving the derivatization of dextran with polymerizable double bonds followed by radical or UV polymerization of the dextran derivatives. Unfortunately, such methods have not been described as providing regioselectivity. Nonetheless, regioselectivity may be necessary to provide highly ordered, swellable and strong hydrogels.

Sugar-based polymers and enzymatic methods of making these sugar-based polymers have been described. See U.S. Pat. No. 5,854,030, to Dordick et al., incorporated herein by reference. Dordick et al. have described sugar-based polymers manufactured by first using enzymatic synthesis to make diacylated sugar intermediates, which can then be polymerized to make sugar-based polymers or hydrogels. The use of both enzymatic and chemical synthesis is known as chemoenzymatic synthesis.

Nonetheless, a need exists for novel hydrogels with tailored three dimensional properties.

### SUMMARY OF THE INVENTION

The invention is based upon the discovery that insoluble, polysaccharides, such as inulin and dextran, can be enzymatically modified in an organic solvent. Thus, in a first aspect, the invention relates to a method for making a high molecular weight polyhydroxy polymer, such as a polysaccharide, inulin or dextran derivative, comprising reacting an acyl donor and the polymer, such as inulin or dextran, to form an acyl ester of the polymer, such as inulin or dextran, in a reaction medium comprising an organic solvent in the presence of a hydrolytic enzyme. In a preferred embodiment, the hydrolytic enzyme is a lipase or a protease, such as an alkaline protease including subtilisin, a bacterial protease, and mixtures thereof. The reaction medium, or organic solvent, preferably solubilizes polymer. Preferred organic solvents include pyridine, dimethylformamide, morpholine, N-methylpyrrolidone and dimethylsulfoxide, particularly anhydrous or dried solvents. In a second aspect of the invention, the invention further relates to a method for making a polymer, such as a polysaccharide, an inulin or dextran polymer, comprising reacting a polymerizable acyl donor and polyhydroxyl polymer in a reaction medium comprising an organic solvent in the presence of a hydrolytic enzyme thereby making a polymeric monomer, such as an inulin monomer, and polymerizing, preferably dimerizing, the monomer, thereby making a novel polymer, such as an inulin polymer.

In a third aspect, the invention further relates to novel products of the processes described herein. Particularly preferred products include a polysaccharide polymer, such as an inulin polymer, characterized by a Degree of Substitution of at least about 10%, a Swelling Ratio at Equilibrium is at least about 2, and/or an average mesh size is between about 10 and 100 Å.

In a fourth aspect, the invention relates to a pharmaceutical composition comprising the novel polymers described herein, optionally, in combination with an active agent, such as a drug and to the use of the novel polymers described herein in methods for the manufacture of a pharmaceutical composition or medicament. In a fifth aspect, the invention relates to a method of delivering an active agent to a patient comprising administering to the patient a pharmaceutical composition described herein. The pharmaceutical compositions are particularly useful for oral delivery of active agents and for delivery of agents to the intestine. Preferred active agents include those to be absorbed in the intestines and/or treat inflammatory bowel disease or Crohn's disease.

In another embodiment, the invention relates to methods of conducting an enzymative reaction in anhydrous DMSO comprising contacting one or more enzymatic substrates, including but not limited to the polyhydroxy polymers and acyl donors described herein, with an alkaline protease, such as those described herein, under reaction conditions thereby conducting an enzymatic reaction. In a preferred embodiment, one or more reaction products are recovered from the reaction medium.

### BRIEF DESCRIPTION OF THE DRAWINGS

FIG. 1 shows conversion and DS as a function of time for the reaction of dextran with VA (the molar ratio of VA to dextran glucopyranose residues was 50%) either in the absence (Δ) or presence of 10 mg/mL of active (□) or thermally deactivated (◯) Proleather or 20 mg/mL of active (7) Lipase, at 50° C. Values were determined by ¹H NMR.

FIG. 2 shows influence of the incubation time of Proleather in DMSO (□) or DMSO plus dextran (◯), at 50° C., in its catalytic performance determined afterwards by the transesterification reaction of dextran with VA (the molar ratio of VA to dextran glucopyranose residues was 50%) for 24 h, at 50° C. Values were determined by titration (average±S.D., n=3).

FIG. 3 shows ¹H (A) and ¹³C NMR (B) spectra of dexT70-VA (DS=31.5%) in D₂O, at 25° C. The denotation x-Sy means that the proton/carbon x is adjacent to a substituted carbon y.

FIG. 4 shows ¹H-¹H COSY spectrum of dexT70-VA (DS=31.5%) in D₂O, at 25° C., showing the signals that are important for the assignment.

FIG. 5 shows ¹H-¹³C HMQC spectrum of dexT70-VA (DS=31.5%) in D₂O at 25° C.

FIG. 6 shows FTIR spectra of (A) dexT70, (B) dexT70-VA (DS=31.5%), and gels obtained from an initial monomer concentration of (C) dexT70-VA (DS=31.5%) of 8% and (D) 30%.

FIG. 7 shows experimental crosslinking density (ρₓ) as a function of the theoretical crosslinking density (ρ_{x,theor}) for dexT70-VA gels with different DS and initial monomer concentrations of 8% (Δ), 20% (◯) and 30% (□). The straight line in the graph connects equal ρ_{x,theor} and ρₓ values.

FIG. 8 shows correlation between ξ and V_{2,s} (between 0.031 and 0.295) for dexT70-VA gels with DS values ranging from 7.2 to 37.0% and obtained from initial monomer concentrations from 8 to 30%. The straight line indicates the linear regression of these data with r²=0.9899.

FIG. 9 shows (A) DSₜₒₜₐₗ and DS_{vinyl} obtained as a function of time for the reaction of DVA with Inulin (molar ratio of DVA to Inulin fructofuranoside residues was 0.5) at a concentration of 6.7% (w/v) in the presence of 10 mg/mL (□), 20 mg/mL(◯) and 40 mg/mL (⋄) Proleather shaken at 250 rpm at 50° C. DS values were determined by ¹H NMR (see text for details). (B) Mₙ and M_{w}/Mₙ as a function of DSₜₒₜₐₗ.

FIG. 10 shows (A) DS as a function of time for the reaction of dexT70 with DVA (initial molar ratio of DVA to dextran glucopyranose residues of 50%) either in the presence of active Proleather FG-F [10 mg/mL (Δ), 20 mg/mL (◯) and 30 mg/mL (□)] and in the absence (∇) or in the presence of thermally deactivated Proleather FG-F (⋄) (10 mg/mL). The arrows in the graph mean that the reaction mixtures gel after that reaction time. (B) Swelling ratio (SR; (M_{w}−M_{d})/M_{d}, where M_{w} is the weight of hydrogel in equilibrium swelling, and M_{d} is the weight of dried hydrogel) measurements of dexT70-DVA hydrogels prepared in the time-course transesterification reactions [Proleather concentration of 20 mg/mL (◯) and 30 mg/mL (□)] and after extensive washing procedures in milli-Q water for 10 days at 4° C. (C,D) SEM micrographs of dexT70-DVA hydrogel surfaces, obtained after 24 h (C) and 72 h (D) (Proleather concentration of 20 mg/mL). Values represent mean and standard deviation (n=3).

FIG. 11 (A,B,C,D) shows scanning electron micrographs of inner regions from the surface of swollen dexT70-DVA hydrogels in 10 mM citrate-phosphate pH 5.0, after being previously dried: DS 28% (A), 30% (B), 27% (C), 29% (D). Hydrogels were obtained either enzymatically (A,B) or chemically (C,D). E) Plot of pore size distribution (log differential intrusion) against diameter of dexT70-DVA hydrogels (in same conditions as previously), with different DS, obtained enzymatically. Average pore diameters (Ø) and porosities (P) are displayed.

FIG. 12 shows A) BSA (◯, ⋄) and lysozyme (□) release profiles in 10 mM PBS pH 7.4 (◯,□) or 10 mM citrate-phosphate pH 5.0 (⋄), at 37° C., from dexT70-DVA DS 31% hydrogels (average±SD, n=3). These networks were previously loaded with protein solutions of 1.25% (w/v, PBS) (◯,□) or 5% (w/v, pH 5.0 buffer) (⋄) for 5 days at 25° C. B) Swelling behaviour of dexT70-DVA DS 31% hydrogels during BSA (◯,⋄) and lysozyme (□) release in the same conditions as above (average+SD, n=3). The swelling index was determined by the ratio of W₁ and W₀ where W₁ is the weight of the hydrogel at time t and W₀ is the initial weight of the hydrogel (after protein loading). C) Relative lysozyme activity as a function of time, when released from dexT70-DVA DS 31% hydrogel in 10 mM PBS pH 7.4, at 37° C. (average±SD, n=3). The starting activity was taken as 100%.

FIG. 13 shows representative light micrographs of dexT70-DVA DS 31% hydrogel implanted subcutaneously and surrounding tissue at different times, stained with hematoxylin/eosin (A), Masson's trichrome (B,C) and periodic acid-schiff (D). (A) At day 2, a moderate inflammatory reaction is observed surrounding the hydrogel (H): granulocytes (arrow), mainly neutrophils, were adhered to the hydrogel and they were surrounded by fibrin (f) and exudate (e). (B) At day 5, a cell layer (arrow) formed essentially by fibroblasts was surrounding the hydrogel, while collagen (c) was underlaying it. Newly blood vessels (v) were also present at the surrounding tissue. Insert shows the adhesion of fibroblasts into hydrogel (hematoxylin/eosin staining). (C) At day 10, several layers of foam cells (fc) were surrounding the hydrogel and they were involved by fibroblast cells and collagen. (D) At day 20, the hydrogel was completely degraded and foam cells (fc) with internalised hydrogel particles were observed at the place originally occupied by the hydrogel. In A: original magnification×100; B and C:×50; D:×250; insert in B:×100.

FIG. 14 shows schematic representation of the direct (A) and indirect (transwell experiment) (B) contact assays. Both assays use growing cells instead subconfluent cells as in the extraction assay.

FIG. 15 shows A) The mitochondrial metabolic activity (mean±SD, n=6) of human skin fibroblasts cultured for 24 h with extracts from 92% (white bars) and 80% (black bars) dexT70-VA hydrogels with different DS values, as determined by the MTT assay (reported as percentage of the negative controls). Hydrogels denoted as extraction indicates that the hydrogels were immersed in citrate-phosphate buffer, 10 mM pH 7.0, for 2 days prior to autoclaving. B) The mitochondrial metabolic activity (mean±SD, n=6) of human skin fibroblasts cultured with dextran, dexT70-VA with different DS values, TEMED and APS, for 24 h, as determined by the MTT assay (reported as percentage of the negative controls). *p<0.01 and **p<0.00¹ in comparison with negative controls.

FIG. 16 shows A) The cell (human skin fibroblasts) proliferation inhibition index (CPII) (mean±SD, n=6) after 72 h for dextran, dexT70-VA with different DS values, TEMED and APS solutions, as determined by the MTT assay. B) The CPII (white bars, mean±SD, n=2) or CPII normalized by diameter of hydrogel (black bars) after 72 h, for dexT70-VA hydrogels with different DS values in direct contact with cells, as determined by the MTT assay. C, D, E and F) Micrographs from phase-contrast inverted light microscopy of human skin fibroblasts cultured for 72 h, under dexT70-VA hydrogels (D,E,F) or in the absence of hydrogel (C). In D, 92% DS 7.2%; E, 80% DS 7.2% and F, 80% DS 12.1%. Original magnification x 200. *p<0.01 and **p<0.001 in comparison with negative controls.

FIG. 17 shows the cell (human skin fibroblasts) proliferation inhibition index (CPII) (mean±SD, n=3) after 72 h for dexT70-VA hydrogels with different DS values in indirect contact with cells (transwell experiment, see text for further details), as determined by a MTT assay.

FIG. 18 shows A) Human skin fibroblasts adhesion (mean±SD, n=3) into dexT70-VA hydrogels with different DS values and initial water contents. Tissue culture polystyrene (TCPS) was used as control. B,C) Micrographs from phase-contrast inverted light microscopy of fibroblast adhesion after culturing for 24 h on TCPS (B) and 80% dexT70-VA DS 12.1% hydrogel (C). Original magnification×200.

FIG. 19 shows representative light micrographs of 80% dexT70-VA DS 7.2% hydrogel implanted subcutaneously and surrounding tissue (perpendicular slice) at different time points, stained with HE (A,B,G,H) and MT (C,D,E,F). Top side is skin; bottom side is underlying muscle (M). (A,B) At day 2, a moderate inflammatory reaction (IR) is seen surrounding the hydrogel (H): a fibrin layer (f) containing lymphocytes (close arrow) and fibroblasts (open arrow) is in contact with the hydrogel, while some exudate (e) is underlying it. (C,D) At day 10, macrophages and fibroblasts (MF) were surrounding the hydrogel (H) and these were evolved by a starting fibrous capsule (fc). (E,F) At day 30, a moderate foreign body reaction is seen surrounding the hydrogel (H): a layer of macrophages and fibroblasts (MF) is seen in the proximity of hydrogel which is surrounded by a fibrous capsule (fc; thickness of ca. 54 μm). (G,H) At day 40, a minimal foreign body reaction is surrounding the hydrogel (H) which is identified by a thin fibrous capsule (ca. 35 μm) with some macrophages (close arrow) and fibroblasts (open arrow). Some blood vessels (V) are also present in the surrounding tissue. In A,C,E and G: original magnification×5; B,F and H:×100; D:×50.

FIG. 20 shows DS obtained as a function of time for the reaction of VA with Inulin (molar ratio of VA to Inulin fructofuranoside residues was 50%) in a concentration of 6.7% (w/v) either in the absence (∇7) or presence of 10 mg/mL (□), 20 mg/mL(◯), 30 mg/mL (Δ) of Proleather as catalyst, or 20 mg/mL of thermally deactivated Proleather (⋄), at 50° C. Values were determined by titration (average±SD, n=3).

FIG. 21 shows Relationship between the theoretical and the obtained DS for Inul-VA as determined by ¹H NMR. The efficiency was calculated as the ratio of the obtained DS to the theoretical DS. FIG. 22 shows GPC chromatograms of Inulin (A) and Inul-VA (B,C,D).

Inul-VA samples were obtained either from original Inulin (B,C) or acetone-precipitated inulin (D) as starting polymers for the transesterification reaction. The DS for Inul-VA samples was 21.2% (B), 34.2% (C) and 17.7% (D).

FIG. 23 shows ¹H (A) and ¹³C (B) NMR spectra of Inul-VA (DS=28.7%) in D₂O, at 25° C.

FIG. 24 shows ₁H-₁H COSY spectrum of Inul-VA (DS=19.3%) in D₂O at 25° C., showing the ¹H-¹H correlations important for the assignment (see text for more details).

FIG. 25 shows ¹H-¹³C HMQC spectrum of Inul-VA (DS=19.3%) in D₂O at 25° C., showing the ¹H-¹³C correlations (see text for more details).

FIG. 26 shows variation of DS at hydroxyl groups (DSᵢ) in positions 6 (◯), 4 (□) and 3 (Δ) with the total degree of substitution in Inul-VA (DS).

FIG. 27 shows solid state CP/MAS ¹³C NMR spectra of Inul-VA (DS=28.7%) in the beginning (A) and after 24 h (B) of the polymerization reaction. The peaks labeled with asterisk represent spinning side bands.

FIG. 28 shows experimental crosslinking density (ρₓ) as a function of the theoretical crosslinking density (ρ_{x,}theor) for Inul-VA gels with same monomer concentration (40%,w/v)

FIG. 29 shows relationship between the mesh size (ξ) and the equilibrium polymer volume fraction (v_{2,s}) for all Inul-VA gels prepared in this work. The straight line indicates the linear regression of the data with r²=0.9923.

### DETAILED DESCRIPTION OF THE INVENTION

The invention is based upon the discovery that polysaccharides, such as inulin and dextran, can be enzymatically modified in an organic solvent.

Thus, in a first aspect, the invention relates to method for making a high molecular weight polyhydroxy polymer, such as a polysaccharide, inulin or dextran derivative, comprising reacting an acyl donor and the polymer, such as inulin or dextran, to form an acyl ester of the polymer, such as an acylated inulin or dextran, in a reaction medium comprising an organic solvent in the presence of a hydrolytic enzyme.

In a preferred embodiment, the hydrolytic enzyme is a lipase or a protease, such as an alkaline protease including subtilisin, a bacterial protease, and mixtures thereof. Examples include an alkaline protease, such as subtlisin and Proleather, bacterial protease, lipase, and mixtures thereof. Hydrolytic enzymes initiate the regioselective diacylation of the sugar molecules with organic acid derivatives. In one embodiment, a regiospecific enzyme is selected.

Hydrolytic enzymes have been found to retain their catalytic activity in organic solvents include, without limitation, Lipozyme, available from Novozymes; Bacterial protease, available under the trade name “Bioenzyme” from GIST-BROCADES; Subtilisin from Bacillus subtilis; Alkaline protease, available under the trade name “Proleather” from AMANO; Bacillus protease available under the trade name “Protease N” from AMANO; Lipase from Candida cylindracea, available from SIGMA; Lipase from porcine pancreas, available from SIGMA; and Lipase from Penicillium Sp., available under the trade name “Lipase G” from AMANO. Although specific to the substantially non-aqueous organic solvent, it should be noted that, as presently understood, the hydrolytic enzymes are non-specific to the acyl donor. A particularly preferred enzyme is Proleather.

The amount of hydrolytic enzyme provided to catalyze the regioselective acylation of polysaccharide is not critical, provided there is sufficient enzyme to initiate the acylation (about 10 mg/ml). By varying the amount of enzyme employed, however, the speed of the acylation can be affected. In general, increasing the amount of hydrolytic enzyme increases the speed of acylation.

The enzyme is preferably used in a dried form, such as a powder, granular form or particulate. Preferably, the enzyme is a lyophilizate or a pH-adjusted lyophilizate. In the instance of Proleather FG-F, it is beneficial to pH-adjust the enzyme to pH 8.0 prior to drying by, for example, flash-freezing in liquid nitrogen followed by lyophilization. Thus, the preferred enzymes are lyophilized or freeze-dried.

The reaction medium, or organic solvent, preferably solubilizes polysaccharide and the acyl donor. Preferred organic solvents include pyridine, dimethylformamide, morpholine, N-methylpyrrolidone and dimethylsulfoxide, particularly anhydrous or dried solvents. In one embodiment, the solubility of polysaccharide in the organic solvent is at least about 1 gram of polysaccharide per liter of solvent at 20° C., preferably between about 1 to 10 grams polysaccharide per liter of solvent at 20° C.

It is advantageous to use anhydrous or dried organic solvents. “Anhydrous” is defined herein to embrace a substantially water-free system. It is understood that a quantity of water is bound to the enzyme. In other embodiments, the water content of the reaction medium is less than about 5% by volume of reaction medium, preferably less than about 1% by volume, more preferably less than 0.25% by volume.

Polysaccharides, as that term is used herein, refer to oligosaccharides and polysaccharides having at least 4 saccharide units. Di- and trisaccharides are not contemplated herein. Preferred polysaccharides include naturally occurring molecules, such as inulin and dextran. Particularly preferred inulins have at least four fructose repeat units. Examples of polysaccharides that can be used include dextran and inulin, and other polysaccharides having a molecular weight of at least about 700 daltons or between about 700 daltons and about 140,000 daltons can be used. Alternatively, other polyhydroxy polymers can be used in the presence invention.

The acyl donor can be any acid, acyl halide, anhydride or ester that will react with the hydrolytic enzyme and the polysaccharide. In a preferred embodiment, the acyl donor will be a carboxylic acid or ester, preferably an aliphatic carboxylic acid or ester. In one preferred embodiment, the aliphatic ester is a methyl ester. However, in some instances, aromatic carboxyl acids, and derivatives thereof, can be used and as well as other acids, such as sulfonic, phosphinic, and phosphoric acids and derivatives can be used. Thus, the acid donor comprises an acyl group and at least one enzymatically-cleaved group, such as an alcohol residue.

A particularly preferred enzymatically cleaved group is a vinyloxy group, as can be found in divinyladipate and vinyl acrylate.

The acyl donor is also characterized by an acyl group. The acyl groups preferably possess a functional group that permits cross-linking between polysaccharides. For example, vinyl-containing acyl donors permit cross-linking via polymerization of the resulting vinyl-substituted polysaccharides. In other embodiments, the acyl donors can contain electrophilic or nucleophilic reactive groups that can be reacted to result in cross-linked polysaccharides. Examples include diamines, dithiols, diacids, aminoacids and mixtures thereof. In a preferred embodiment, the acyl group is a polymerizable moiety, such as an alkenyl or alkadienyl group. Preferred acyl donors include vinyl acrylate and vinylmethacrylate. In another embodiment, the acyl donor reacts with two molecules of polysaccharide, thereby cross-linking the polysaccharide without a subsquent reaction. In this embodiment, preferred acyl donors include are esters of di and tricarboxylic acids, such as citric acid, citrate esters, adipic acid, adipate esters and other bis carboxylic acids and esters. A particularly preferred acyl donor is divinyl adipate.

Preferably, the reaction mixture can be agitated, for example, at about 100-300 rpms in an orbital shaker or by other means. Often, the enzymatic reactions can be run at a temperature of from about 10° C. to about 60° C. The reaction can be conducted until the desired degree of acylation is achieved. Often, the reaction can be terminated between about 12 and 48 hours.

Once the reaction is complete, the reaction is terminated. Termination can be accomplished by inactivating the enzyme or by removing the enzyme, the latter being preferred. Methods of separating enzymes are generally known. Where the enzyme is present in the reaction medium as a solid, the enzyme can be removed by centrifugation, filtration or other means.

It is preferred that the enzymatic transesterification reaction is regiospecific. The polysaccharide derivative so obtained can be used as is or can be further purified, also by known methods.

In a second aspect, the invention further relates to a method for making a polymer, such as a polysaccharide, an inulin or dextran polymer, comprising reacting a polymerizable acyl donor and polyhydroxyl polymer in a reaction medium comprising an organic solvent in the presence of a hydrolytic enzyme thereby making an polymeric monomer, such as an inulin monomer, and polymerizing, preferably dimerizing, the monomer, thereby making a novel polymer, such as an inulin polymer.

In preparing the hydrogels of the present invention, the acylated polysaccharide can polymerized by mixing the polysaccharide olefinic monomer with a free-radical polymerization initiator. Any suitable free-radical initiator is contemplated for use in the present invention. Examples of suitable initiators ammonium persulfate in water, t-butyl peroxide, benzoyl peroxide or azobisisobutyrol-nitrile (AIBN) in non-aqueous solvents. Preferably, the initiator will comprise AIBN. The reaction conditions used herein are, generally, those known in the art. In one embodiment the polymerizable acyl donor comprises two terminally located vinyl groups.

As above, the polymers thus provided can be used as is or can be further purified by, for example, precipitation purification, washing and other methods.

In a third aspect, the invention further relates to novel products of the processes described herein. Particularly preferred products include a polysaccharide polymer, such as a crosslinked inulin or dextran polymer. The polymer is preferably characterized by a Degree of Substitution of at least about 5%, preferably at least about 10%, more preferably between about 10% and 50%. The DS is defined by:

$$D S = \frac{7 * x}{y} * 100$$

where x is the average integral of the protons from vinyl group (δ6.44-6.00 ppm) and y is the integral of all dextran protons (between 5.50 and 3.10 ppm).

Preferably, the polymers alternatively or additionally possess a Swelling Ratio at Equilibrium of at least about 2, preferably at least about 3, more preferably between about 3 and 40.

The swelling ratio at equilibrium (SRE) can be calculated according to:

$$SRE = \frac{{W}_{s} - {W}_{d}}{{W}_{d}}$$

Wherein, W_{d} is the dried weight and Wₛ is the swelled weight.

Preferably, the polymers alternatively or additionally possess an average mesh size is between about 10 and 100 Å, preferably between about 20 and 50 Å.

In yet another embodiment, preferred polymers are characterized by a Molecular Weight between crosslinks of at least about 300 g/mol, preferably between about 300 g/mol and 20,000 g/mol, and more preferably between about 500 g/mol and 5000 g/mol. One preferred polymer is a cross-linked inulin characterized by a DS of about 10%, an SRE of about 2, and a mesh size of about between 10-100 Angstroms whereby the polymer is crosslinked by a diester or whereby the polymer is crosslinked with a dimerized vinyl acrylate.

In a fourth aspect, the invention relates to a pharmaceutical composition comprising the novel polymers described herein, optionally, in combination with an active agent, such as a drug and to the use of the novel polymers described herein in methods for the manufacture of a pharmaceutical composition or medicament. One preferred pharmaceutical composition comprises an active agent and a cross-linked inulin polymer characterized by a DS of about 10%, an SRE of about 2, and a mesh size of about between 10-100 Angstroms. Dosages of active agents can be in the range of about 1-50% (w/w) active agent to total solids, preferably about 5-30%, and more preferably between about 5-10%.

In a fifth aspect, the invention relates to a method of delivering an active agent to a patient comprising administering to the patient a pharmaceutical composition described herein. The pharmaceutical compositions are particularly useful for oral delivery of active agents and for delivery of agents to the intestine. Preferred active agents include those to be absorbed in the intestines and/or treat inflammatory bowel disease or Crohn's disease.

Active agents that can be used in the claimed invention include, without limitation, analgesics; muscle relaxants; antacids; antihistamines; decongestants; anti-inflammatories; antibiotics; anti-virals; oral vaccines; probiotics; cancer chemotherapies; antimycotics; oral contraceptives; diuretics; antitussives; anesthetics; bioengineered pharmaceuticals; insulin; psycotherapeutic agents; hormones; cardiovascular agents; vitamins; minerals; small molecules; nucleic acids; proteins;

viral particles and nutraceuticals. In a particularly preferred embodiment, the active agent can be one or more cells or a tissue culture, such as in an implant at the site of a wound.

The active agents can be absorbed into or adsorbed onto the polymers described herein. In one embodiment, the active agent is dispersed within the polymer.

In a sixth aspect of the invention, it can be advantageous to conduct the polymerization reaction in the presence of the active agent, thereby physically entrapping the agent within the polymer network.

The pharmaceutical composition in accordance with the fourth aspect of the invention can be admininistered parenterally or enterally. Preferred methods of parenteral administration include administration of an injection containing the described polymeric composition in an injectable medium. Alternatively, the composition can be administered topically, such as by a cream, ointment or patch, at a wound site. Where the polymer hydrogel is selected to protect the drug, or inhibit absorption by the GI tract until the intestine, the route of administration can be enteral, such as an oral administration via, for example, a tablet or capsule.

The methods of the invention are particularly well suited for the targeted delivery of an active agent for absorption or action in the intestine, such as in the treatment or prevention of inflammatory bowel disorder or Crohn's disease.

### EXEMPLIFICATION

### Example 1

### Enzymatic Synthesis of Dextran-containing Hydrogels Using Biocatalysis nd Free Radical Polymerization

Overview

This Example describes a novel strategy to prepare dextran gels using a chemoenzymatic two-step procedure. In the first step, dextran was enzymatically derivitized with vinyl acrylate (VA). Since this polysaccharide is soluble only in the most polar organic solvents, the enzymatic reaction was carried out in dimethylsulfoxide (DMSO). Suprisingly, despite reports in the literature [15, 16] that showed the absence of enzyme activity in DMSO, this Example shows that DMSP supported the catalytic activity of “Proleather” FG-F and lipase AY, a protease and lipase from bacillus sp. And Candida rugosa, respectively. Furthermore the benefits of the enzyme-catalyzed system were evaluated by comparison to a similar chemical approach. In the second step, aqueous solutions of dextran acrylate were converted to hydrogels upon free radical polymerization. Gels with different equilibrium swelling ratios and physical properties were obtained. These dextran-based hydrogels may have special use as drug delivery matrices for colonic targeting, particularly because of their expected degradation by dextranases, which are known to be present in the colon [17].

Materials and Methods

Materials

The protease Proleather FG-F and lipase AY were generous gifts from Amano Enzyme Co. (Troy, Va., USA). Dextran (from Leuconostoc mesenteroides, dexT70, Mₙ=39940, M_{w}=70,000, according to the manufacturer's specification) was obtained from Fluka Chemie AG (Buchs, Switzerland). Vinyl acrylate (VA), 4-dimethylaminopyridine (4-DMAP), DMSO, N,N,N′,N′-tetramethylenediamine (TEMED), and ammonium persulfate (APS) were purchased from Aldrich (Milwaukee, Wis., USA). DMSO was dried over 3 Å molecular sieves overnight before use. Regenerated cellulose dialysis tubes with a MWCO of 50,000 Da were purchased from Spectrum (CA, USA). All other chemicals and solvents used in this work were of the highest purity commercially available.

Analytical Methods

¹H and ¹³C NMR spectra were recorded on a Varian Unity spectrometer (Palo Alto, Calif.) at 300 MHz and 75 MHz, respectively. ¹H NMR spectra were recorded in D₂O (60-100 mg in 0.7 mL) using a pulse angle of 90° and a relaxation delay of 30 s. The water signal, used as reference line, was set at δ 4.75 ppm and was suppressed by irradiation during the relaxation delay. The number of scans in the spectral acquisition was 16. ¹³C NMR spectra were recorded in D₂O using a pulse of 30° and relaxation delay of 1 s, and tert-butanol (tb) was used as reference and set at δ 31.2 ppm versus tetramethylsilane. Generally, the number of scans was 16,000. For quantitative ¹³C NMR, the decoupler was gated on during acquisition and off during delay, to suppress the nuclear Overhauser effect. The spectra were recorded in D₂O using a pulse of 90° and relaxation delay of 30 s. Bi-dimensional spectra were recorded on a Varian Unity 500 MHz spectrometer (Palo Alto, Calif.). ¹H-¹H COSY spectra were collected as a 1,024×416 matrix covering a 2,500 sweep width using 64 scans/increment. Before Fourier transformation, the matrix was zero filled to 2,048×2,048 and standard sine-bell weighting functions were applied in both dimensions. ¹H-¹³C HMQC spectra were collected as a 1,024×512 matrix covering sweep widths of 2,500 Hz and 11,500 Hz in the first and second dimensions, respectively, and using 64 scans/increments. Before Fourier transformation, the matrix was zero-filled to 2,048×2,048 and standard gaussian weighting functions were applied in both dimensions.

FTIR spectra were recorded with a Nicolet Magna-IR 550 spectrometer (Madison, Wis.). The dry samples were powdered, mixed with KBr, and pressed into pellets under reduced pressure. The FTIR spectra were obtained by recording 128 scans between 4000 and 450 cm⁻¹ with a resolution of 2 cm⁻¹.

Gel permeation chromatography (GPC) was performed with a Knauer WellChrom Maxi-Star K-1000 equipped with a Perkin Elmer LC-25 RI detector refractive index detector and three PL (Polymer Laboratories Inc., MA, USA) series columns (PL aquagel-OH Guard 8 mm, 50×7.5 mm, precolumn; and two PL aquagel-OH 40, 8 mm, 300×7.5 mm, with a exclusion limit of 2×10⁵). The eluent was 10 mM NaCl in Milli Q water at a flow rate of 1 mL/min. A calibration was obtained with dextran standards of narrow polydispersity in the molecular weight range from 11,600-147,600 Da. The GPC chromatograms were obtained from samples dissolved in 0.01 M NaCl in a concentration of 20 mg/mL.

The determination of the degree of substitution (DS, the amount of acrylate groups per 100 dextran glucopyranoside residues) by titration was performed according to Vervoort et al. [18]. Dext70-VA samples (50 mg) were dissolved in 0.1 N NaOH (4 mL) and stirred for 72 h, at 20° C., to obtain alkaline hydrolysis of the ester. The molar consumption of NaOH was determined by back titration with 0.1 N HCl after adding 2 drops of phenolphthalein solution as indicator.

Preparation of “Proleather” and Lipase

“Proleather” and lipase were “pH-adjusted” in the presence of 20 mM phosphate buffer at pH 8.0 and 7.5, respectively, following the procedure by Klibanov [19]. After flash-freezing in liquid nitrogen, the samples were lyophilized on a freeze drier (Labconco Corp., Kansas City, Mo.) for 48 h. The water contents of the lyophilized powders were determined with a Mettler LJ16 Moisture Analyzer (Mettler-Toledo AG, Switzerland) to be 5.6 and 7.9% (w/w) for Proleather and Lipase, respectively.

Thermally deactivated Proleather and Lipase were prepared by suspending the enzymes (10 mg/mL and 34 mg/mL for Proleather and Lipase, respectively) in 250 mL of 20 mM phosphate buffer pH 8.0 in a 500 mL round-bottomed flask fitted with a water-cooled condenser. The enzyme solutions were refluxed for 5 h, after which they were allowed to cool to room temperature, and the water removed by freeze drying. The proteolytic activity of Proleather and its thermally deactivated form were determined according to the manufacturer's specification based on the hydrolysis of casein, with a unit (U) defined as the amount of enzyme required to release 1 μg of tyrosine per min. The activities were 1.31±0.10 and 0.11±0.01 U per mg of enzyme, for active and deactivated Proleather, respectively. The hydrolysis activity of thermally deactivated Lipase were determined using glycerol trioleate as substrate [20], with a unit (U) defined as the amount of enzyme which releases 1 μmol of fatty acid per minute. The activities were 9.05±1.42 and 0.86±0.77 U per mg of enzyme, for active and deactivated Lipase, respectively.

Proleather was also pre-inactivated using phenylmethanesulfonyl fluoride (PMSF): 300 mg of enzyme were dissolved in phosphate buffer (20 mM, pH 8.0) and 0.2 mL of PMSF solution (1.74%, w/v, in ethanol) was added. The solution was shaken at 25° C. and 200 rpm for 24 h and then lyophilized.

Determination of Protein Solubility in DMSO

The concentration of Proleather and lipase dissolved in DMSO was determined by the BCA assay (Pierce, Rockford, Ill.). Enzymes were placed in the solvent and agitated for 1 h at 50° C., and undissolved particles were removed by centrifugation (10 min, 5000 rpm). Afterwards, the solutions were diluted 10× with water. In every case, it was confirmed that the residual solvent did not affect the assay. A calibration curve was prepared using BSA standards of known concentrations. The original protein content in 100 mg of enzyme powder was 16.26±0.50 and 6.06±0.11 mg for Proleather and Lipase, respectively.

Enzymatic Synthesis of dexT70-VA

Dextran (10 g) and a calculated amount of VA (0.60-3.01 g) were dissolved in DMSO (150 mL) and the reaction initiated by adding 1.5 g of “pH-adjusted” Proleather. The reaction mixtures were shaken at 50° C. (250 rpm) in a temperature-controlled New Brunswick Scientific C24 orbital shaker (Edison, N.J., USA) for 72 h, after which they were centrifuged at 4000 rpm for 10 min. The supernatants were precipitated in acetone (700 mL) and further centrifuged at 4000 rpm for 5 min. The precipitates were dissolved in water and dialyzed for 5 days against HCl aqueous solution, pH 3.0, and two more days against milli-Q water, at 4° C. Finally, the aqueous solutions were lyophilized for 48 h yielding a fluffy product (isolated yields of 45-56%).

In the lipase-catalyzed synthesis of dexT70-VA, dextran (1 g) and VA (0.301 g) were dissolved in DMSO (15 mL) and the reaction commenced by adding 300 mg of “pH-adjusted” enzyme. After reaction at 50° C. for a certain period of time, the reaction mixture was centrifuged (4000 rpm, 10 min) and the supernatant was dissolved in milli-Q water and dialyzed in the same conditions as stated before. After lyophilization the isolated yields were ca. 80%.

Reaction time courses of dexT70-VA synthesis either in the presence of active or thermally deactivated Proleather (10 mg/mL) or in the absence of enzyme were performed independently in 15 mL of anhydrous DMSO containing dextran (1 g) and VA (0.301 g) at 250 rpm and 50° C. The purification of the products was performed as described for the lipase synthesis of dexT70-VA.

To assess the stability of Proleather in DMSO or in DMSO plus dextran (1 g), the enzyme (150 mg) was incubated in each of these solutions (15 mL) for a specific time (up to 48 h) and then the acylation reaction initiated by adding dextran (1 g) and VA (0.301 g) or solely VA (0.301 g), respectively. The reaction mixtures were shaken at 50° C. (250 rpm) for 24 h, after which they were centrifuged at 4000 rpm for 10 min. The purification of the reaction mixtures was performed as described above.

Chemical Synthesis of dexT70-VA

Dextran (1 g) and VA (0.181 g) were dissolved in DMSO (15 mL) and the reaction commenced by adding 4-DMAP (200 mg). The reaction mixtures were shaken at 50° C. (250 rpm) for 72 h. The reactions were stopped by adding an equimolar amount of concentrated HCl to neutralize the 4-DMAP. The reaction mixtures were dialyzed for 10 days against milli-Q water at 4° C. Afterwards, the solution was lyophilized yielding 0.774 g (yield: 68.6%) of product. Dext70-VA (DS 11.6%). ¹H NMR (δ, D₂O, ppm): 8.02 (m, 2H, 4-DMAP), 6.83 (m, 2H, 4-DMAP), 6.46 (m, 1H, CH═CH₂), 6.22 (m, 1H, CH═CH₂), 6.01 (m, 1H, CH═CH₂), 5.42-5.12 (m, 3H, H1 in α-1,3 linkages, H1-S2 and H3-S3), 5.00 (d, 1H, H1-S3), 4.94 (d, 1H, H1) 4.10-3.80 (m, 2H, H6′ and H5), 3.78-3.60 (m, 2H, H6″ and H3), 3.60-3.30 (m, 2H, H2 and H4), 3.15-3.08 (m, 6H, 4-DMAP). ¹³C NMR (6, D₂O, ppm): 173.6-173.0 and 169.6-168.8 (C═O), 158.0 (4-DMAP), 143.1 (4-DMAP), 135.3 and 134.6 (CH₂═CH), 128.9 and 128.6 (CH₂═CH), 109.1 (4-DMAP), 99.2 (C1), 96.5 (C1-S2), 77.7 (C3-S3), 74.9 (C3 and C2-S2), 72.9 (C2), 72.6 (C3-S2), 71.7 (C5), 71.1 (C2-S3 and C4), 69.0 (C4-S3), 67.1 (C6). FTIR (KBr, cm⁻¹): 3391 (V_{O-H}), 2929 (V_{CH2}), 1727 (v_{C=O}), 1655 (VC_{N}⁺, 4-DMAP), 1574 (V_{COO}).

Preparation of dexT70-VA Hydrogels

DexT70-VA gels were obtained by free radical polymerization of aqueous solutions of dexT70-VA as a function of DS and monomer concentration. DexT70-VA (160, 400, and 600 mg) was dissolved in 1.8 mL of 0.2 M phosphate buffer, pH 8.0, and bubbled with nitrogen for 2 min. The polymerization reactions, performed in a closed plastic tube (diameter≈1.5 cm), were initiated by adding 100 μL APS (80 mg/mL in 0.2 M phosphate buffer, pH 8.0) and 100 μL TEMED solution (13.6% (v/v) in water; pH adjusted to 8.0 with 12 N HCl), and allowed to proceed for 24 h at 25° C. The gels were subsequently removed from the tube and immersed in ca. 50 mL of 0.01 M citrate-phosphate buffer, pH 7.0, for 5-15 days, changing the buffer daily, at 25 or 37° C. At regular intervals, the swollen gels were removed, blotted with filter paper to remove surface water, weighed, and returned to the same container until weight stabilization was observed. The gels were then dried at room temperature, under vacuum, in the presence of phosphorous pentoxide, and weighed to determine the dried weight, W_{d}. The swelling ratio at equilibrium (SRE) was calculated according to Eq. 1:

$$SRE = \frac{{W}_{s} - {W}_{d}}{{W}_{d}} \qquad ( 1 )$$

The molecular weight between the crosslinks (\overline{M}_{c}) was calculated from the equilibrium swelling theory of Flory and Rehner [21], modified by Peppas et al. [22] for the case of networks where the crosslinks were introduced in solution, according to Eqs. 2 and 3:

$$\begin{matrix}\frac{1}{{\overline{M}}_{c}} = \frac{2}{{\overline{M}}_{n}} - \frac{( \frac{\overline{v}}{{V}_{1}} ) [ ln ( 1 - {v}_{2 , s} ) + {v}_{2 , s} + {{χ}_{1} ( {v}_{2 , s} )}^{2} ]}{{v}_{2 , r} [ {( \frac{{v}_{2 , s}}{{v}_{2 , r}} )}^{1 / 3} - 0.5 ( \frac{{v}_{2 , s}}{{v}_{2 , r}} ) ]} & ( 2 ) \\ \frac{1}{{\overline{M}}_{c}} = \frac{2}{{\overline{M}}_{n}} - \frac{{( \frac{\overline{v}}{{V}_{1}} ) [ ln ( 1 - {v}_{2 , s} ) + {v}_{2 , s} + {{χ}_{1} ( {v}_{2 , s} )}^{2} ] [ 1 - \frac{1}{{χ}_{c}} {( \frac{{v}_{2 , s}}{{v}_{2 , r}} )}^{2 / 3} ]}^{3}}{{{v}_{2 , r} [ {( \frac{{v}_{2 , s}}{{v}_{2 , r}} )}^{1 / 3} - 0.5 ( \frac{{v}_{2 , s}}{{v}_{2 , r}} ) ] [ 1 + \frac{1}{{χ}_{c}} {( \frac{{v}_{2 , s}}{{v}_{2 , r}} )}^{1 / 3} ]}^{2}} & ( 3 )\end{matrix}$$

where \overline{Mₙ} is the number average molecular weight of dextran (39,940 Da), \overline{v} is the partial specific volume of dextran (0.62 cm³/g) [23], V₁ is the molar volume of water (18 cm³/g), χ_{c} is the Flory polymer-solvent interaction parameter (0.473 for dextran/water) [24], χ_{c} is the number of links of the chain (χ_{c}=2M_{c}/Mᵣ, where Mᵣ is the molecular weight of the dextran repeating unit, 162.14), V_{2,r} is the polymer fraction of the gel after gel formation and v_{2,s} is the polymer fraction at equilibrium swelling. v_{2,r} and v_{2,s} were calculated from the weight of the gels before exposure to the buffer solution and after equilibrium swelling, respectively, assuming volume additivity of water and dextran. The average mesh size was calculated according to [14]. The crosslinking density, ρₓ, was determined from \overline{M}_{c} calculated from Eq. 4 [22]:

$${ρ}_{x} = \frac{1}{\overline{v {M}_{c}}}$$

The theoretical crosslinking density was also calculated from Eq. 4, nevertheless the theoretical number average molecular weight between crosslinks (\overline{M}_{c, theor}) was calculated from Eq. 5:

$${\overline{M}}_{c , theor} = \frac{M r \times 100}{D S} \qquad ( 5 )$$

Results and Discussion

Enzymatic Synthesis of dexT70-VA

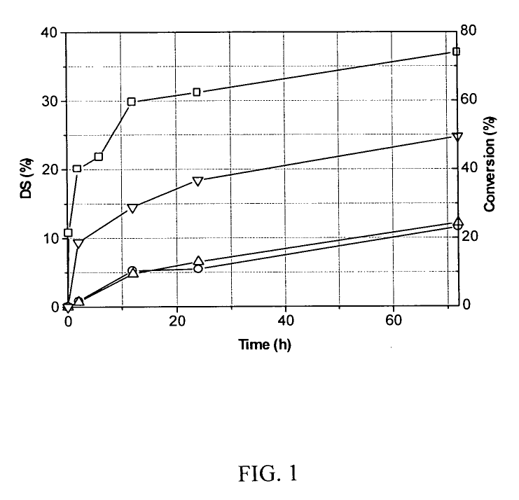

Conventional wisdom holds that enzymes are inactive in nearly anhydrous DMSO [15,25], and that such inactivity may be a direct result of protein solubilization in the organic milieu [26,27], which causes deleterious changes in the proteins' secondary and tertiary structures. Indeed, both Proleather and C. rugosa lipase are partially soluble in DMSO: 200 mg/mL of crude enzyme (corresponding to a protein concentration of 32.52 and 12.12 mg/mL of Proleather and lipase, respectively) initially suspended in DMSO resulted in soluble protein concentrations of 30.61+0.40 and 3.80±0.20 mg/mL for Proleather and lipase, respectively. Despite the solubilization of Proleather, the enzyme remained catalytically active in nearly anhydrous DMSO, as shown from the time-course reaction of dexT70 with VA either in the presence of active or thermally-deactivated Proleather or absence of enzyme (FIG. 1). In the presence of active Proleather, monomer conversion reached ca. 22 and 60% after 15 min and 12 h of reaction time, respectively. Thus, after 12 h, the turnover of the enzyme was ca. 4700 (mol dextran acylated on a per glucose moiety basis per mol enzyme present in the reaction mixture). In contrast, for the same time periods, either in the absence of enzyme or in the presence of thermally-deactivated enzyme, much lower conversions were obtained (0 and 10% for 15 min and 12 h of reaction time, respectively). We also evaluated the reactivity of PMSF-inactivated Proleather. After 12 h, the conversion of dextran with VA was ca. 14% (data not shown). This was a bit higher than the thermally deactivated enzyme, but far lower than in the presence of active enzyme. These results strongly suggest that the dextran acylation in DMSO was enzymatic. Similar results were obtained with C. rugosa lipase. In the presence of active Lipase, the efficiency of the coupling reaction was lower than that obtained by Proleather, however higher than the results obtained in the absence of enzyme or in the presence of thermally deactivated enzyme (DS of 14% in 72 h [Note; there is no reliable active site inhibitor of the lipase]). Interestingly, the Proleather-catalyzed reaction appears to stop after ca. 12 h, as noted by the slopes in FIG. 1, which are identical for the enzymatic reactions and the controls. This can be ascribed to the likely inactivation of the enzyme during incubation in DMSO.

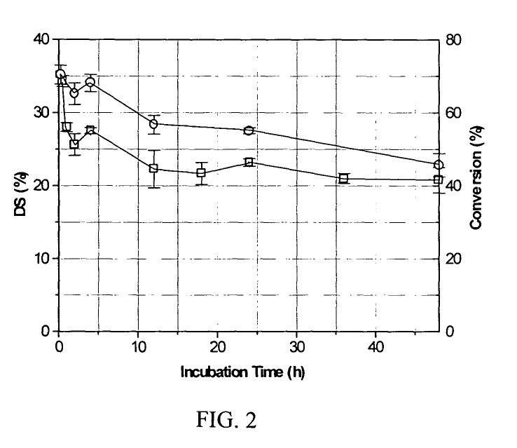

Enzyme activity and stability can be improved by adding polymers (e.g. polystyrene and ethylcellulose) into the reaction medium [28]. Such effects were ascribed either to the formation of complexes between enzyme and polymer or to the control of the water activity by the polymer, which leads to increased enzyme activity and stability [28]. We envisioned a similar “protection” mechanism for Proleather-catalyzed transesterification of dextran. To that end, Proleather was incubated either in DMSO or DMSO plus dextran as a function of time, followed by measuring the transesterification activity of the enzyme in 24 h reactions. In the absence of dextran, the enzyme activity dropped within the first 12 h of incubation, after which it remained unchanged up to 48 h (FIG. 2). In the presence of dextran, the stability is slightly improved (ca. 20%) up to 12 h and then decreases almost to the same level as depicted in FIG. 2. These results show that dextran promotes only a modest degree of enzyme stabilization in DMSO; however, the “protection” effect is not a dominant explanation for the catalytic performance of Proleather in DMSO.

Preparative-scale reactions for the synthesis of dexT70-VA with different DS values were performed in the presence of 10 mg/mL Proleather for 72 h, at 50° C. Table 1 presents the DS values obtained for dexT70-VA samples and the respective isolated yields. Most of the VA was attached to dextran (efficiency>71%) and that it was possible to control the DS by varying the molar ratio of VA to dextran (Table 1). Furthermore, reasonable yields were also obtained (>45%) using polymer purification involving an acetone precipitation step followed by dialysis.

TABLE 1
Degree of substitution obtained and isolated
yields for dexT70-VA monomers.

| | Theoretical | Obtained | Efficiency^{c} | Isolated yield |
|---|---|---|---|---|
| Entry | DS^{a} (%) | DS^{b} (%) | (%) | (%) |
| 1 | 10 | 7.2 | 71.4 | 55.6 |
| 2 | 20 | 15.1 | 75.7 | 47.8 |
| 3 | 30 | 22.4 | 74.6 | 50.6 |
| 4 | 40 | 31.5 | 78.9 | 45.3 |
| 5 | 50 | 37.0 | 74.1 | 47.3 |

^{a}Calculated as molar ratio of VA to dextran glucopyranose residues (×100).
^{b}Determined by ¹H NMR and calculated according to eq.6 (see text).
^{c}Calculated as the ratio of the obtained to the theoretical DS (×100).

GPC analyzes of dexT70 and dexT70-VA samples showed that the dextran peak was shifted to higher molecular weights with the introduction of acrylate groups in the dextran backbone and the enzyme did not degrade the dextran during the derivatization reaction (data not shown).

Characterization of dexT70-VA Obtained Enzymatically by NMR Spectroscopy

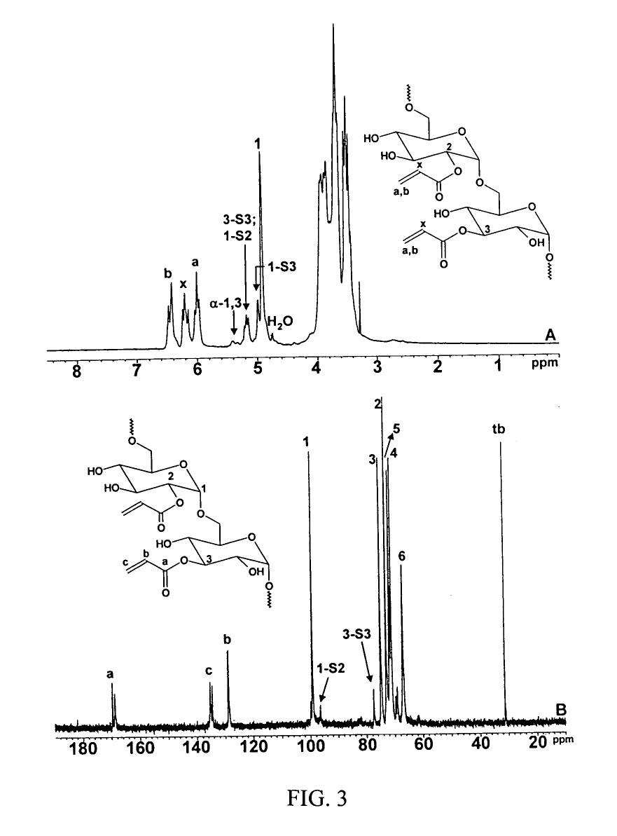

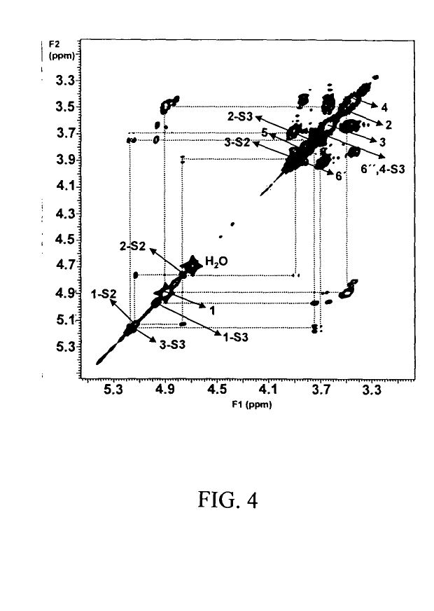

The ^{1H} and ¹³C NMR spectra of dexT70-VA are depicted in FIG. 3. In the ¹H NMR spectrum (A) the peaks between δ 5.50 and 3.10 ppm are attributed to protons of dextran with their assignments clearly shown by the ¹H-¹H COSY displayed in FIG. 4. Furthermore, the signals from the acrylate groups attached to the dextran backbone are observed at δ 6.00, 6.19 and 6.44 ppm. The DS was calculated using Eq. 6:

$$DS = \frac{7 * x}{y} * 100 \qquad ( 6 )$$

where x is the average integral of the protons from vinyl group (δ 6.44-6.00 ppm) and y is the integral of all dextran protons (between 5.50 and 3.10 ppm).

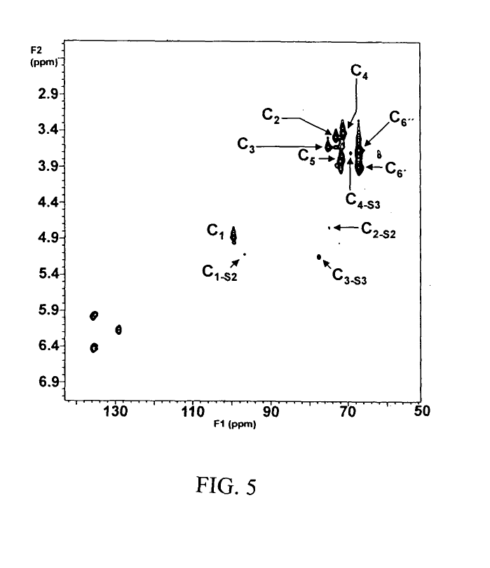

The synthesis of dexT70-VA was also confirmed by ¹³C NMR spectroscopy (FIG. 3B). The glucopyranosyl and acrylate carbons are displayed in the range of 99.9-67.1 and 169.7-128.7 ppm, respectively. All the signals from the acrylate group are doublets (Cₐ:169.7 and 168.8 ppm, C_{c}: 135.3 and 134.6 ppm, C_{b:130.0} and 128.7 ppm), which indicate the presence of two different positional isomers in dexT70-VA. The ester positions on the glucopyranosyl residues were determined [29] based on the additional signals presented in the ¹³C NMR spectrum (FIG. 3B) of dexT70-VA that varied from δ 99.9 to 67.1 ppm, and further confirmed by ¹H-¹H COSY (bi-dimensional NMR experiment showing the correlations among the protons) and ¹H-¹³C HMQC NMR (bi-dimensional NMR experiment showing the correlations between protons and carbons) spectra displayed in FIGS. 4 and 5, respectively.

The two positional isomers in the main dextran backbone are at positions 2 and 3 in the glucopyranosyl residues, and the respective ¹³C-NMR assignments are presented in Table 2. The confirmation of these two positional isomers comes from the ¹H-¹³C HMQC NMR spectrum (FIG. 5) where the ¹³C peaks at 74.6 ppm (modification at 2-position) and 77.4 ppm (modification at 3-position) are correlated with ¹H signals at 4.75 and 5.16 ppm, respectively. From ¹H-¹H COSY (FIG. 4) the signal at 4.75 has two cross-peaks at 5.14 ppm and 3.89 ppm corresponding to the vicinal protons at 1 and 3-positions, respectively, while the signal at 5.16 ppm has two cross-peaks at 3.75 and 3.69 ppm corresponding to the vicinal protons at positions 2 and 4, respectively. According to the ¹³C quantitative NMR results, the positional isomer ratio in dexT70-VA with DS of 31.5% is 43:57 at positions 2 and 3, respectively. These results indicate that the reactivity of hydroxyl groups at position 2 and 3 is nearly identical. No evidence was observed in the ¹H-¹H COSY spectrum of shared cross-peaks with both positional isomers, indicating that all the glucopyranosyl residues modified are mono-substituted. As a consequence, the relative reactivity of the hydroxyl groups is not influenced by substitution of other hydroxyl groups in the same glucopyranoside unit.

TABLE 2
¹³C NMR assignments of the glucopyranosyl ring carbons (δ, ppm) on
dexT70-VA with DS 31.5%.

| | dexT70-VA | |
|---|---|---|
| dexT70 | 2-substituted | 3-substituted |

| | Obs. | Obs. | | Obs. | |
|---|---|---|---|---|---|
| Carbon | signal | signal | Δδ | signal | Δδ |
| 1 | 99.2 | 96.4 | −2.8 | 99.2 | |
| 2 | 72.9 | 74.6 | +1.7 | 71.2 | −1.7 |
| 3 | 74.9 | 72.6 | −2.3 | 77.4 | +2.5 |
| 4 | 71.1 | 71.1 | | 69.0 | −2.1 |
| 5 | 71.7 | 71.7 | | 71.7 | |
| 6 | 67.0 | 67.0 | | 67.0 | |

Candida rugosa lipase was also examined for its ability to catalyze the transesterification of dextran with VA to assess whether any changes in the substitution pattern could occur by an enzyme from a different source. The results obtained by NMR showed that the substitution pattern changed slightly. Two positional isomers at positions 2 and 3 were obtained; however, the regioisomer at position 3 was more highly favored (a ratio of 28:72 for isomers at positions 2 and 3, respectively). The results obtained seem to indicate that both enzymes could not distinguish perfectly the two secondary hydroxyl groups at 2 and 3 positions. Nonetheless, the regioselectivity of the lipase demonstrates clear enzymatic transformation, when compared to the chemical route (see below).

Dextran is composed by glucopyranoside residues as repeating units “protected” at positions 1 and 6, and therefore, lacks primary hydroxyl groups. For this reason, it is instructive to compare the substitution pattern achieved by Proleather- and Lipase-catalyzed transesterification of dextran with the results reported in the literature for enzyme-catalyzed transesterification reactions involving protected glucose molecules. Therisod and Klibanov [30] reported that the Candida cylindracea lipase (reclassified as C. rugosa lipase)-catalyzed transesterification of 6-O-butyrylglucose with 2,2,2-trichoroethylbutyrate in tetrahydrofuran displayed comparable reactivity toward the C-2 and C-3 hydroxyls (a ratio of ca. 60:40). Acylation at C-2 and C-3 was also reported by Macmanus and Vulfson [31] for the Pseudomonas cepacia lipase-catalyzed transesterification of 6-O-trityl-D-glucose with vinyl acetate (acyl donor and solvent) to give a C-2:C-3 ratio of 85:15. Hence, the enzymatic substitution pattern achieved for dexT70-VA using Proleather and Lipase, favoring the synthesis of the regioisomer at position 3 to the one in position 2, is somewhat different from those previously reported for protected glucose molecules. This might be due to the nature of the acylating agent used [32], the nature of DMSO as opposed to more nonpolar solvents, or to the specific 3-D architecture of dextran in solution, which may favor the acylation at position 3, or to a combination of each of these factors.

Chemical Synthesis of dexT70-VA

Dextran was also modified chemically with VA using 4-DMAP as catalyst in DMSO at 50° C., following a methodology previously reported by van Dijk-Wolthuis et al. [9]. The results obtained by NMR spectroscopy showed that the incorporation efficiency of VA in the dextran backbone was 38.6%; lower than that found in the reaction using Proleather and Lipase as catalysts. Furthermore, traces of the 4-DMAP were observed after extensive dialysis (10 days); representing ca. one molecule of 4-DMAP for each acrylate group attached to dextran. This highlights the difficulty in removing the base catalyst from the reaction mixture. Nevertheless, the dexT70-VA synthesized using 4-DMAP as catalyst showed a similar substitution pattern (the ratio of isomers at positions 2 and 3 was 53:47) as that achieved by Proleather, although clearly distinct from lipase catalysis.

Preparation and Characterization of dexT70-VA Gels

The acrylate groups in dexT70-VA were polymerized using APS and TEMED as free radical initiators. Aqueous solutions of dexT70-VA in several concentrations (8, 20 and 30% (w/v)), yielding different DS values, were polymerized at 25° C., and gelation was observed within ca. 5 min. The appearance of the resulting hydrogels was different depending on the DS and the initial dexT70-VA concentrations. Specifically, hydrogels obtained from initial dexT70-VA concentrations of 20 and 30% (w/v) were generally transparent; however, the opacity increases when DS increases. When the initial dexT70-VA concentration was 8% (w/v), the hydrogels obtained were transparent when DS values ranged from 7.2 to 15.1% and opaque for DS values higher than 15.1%.

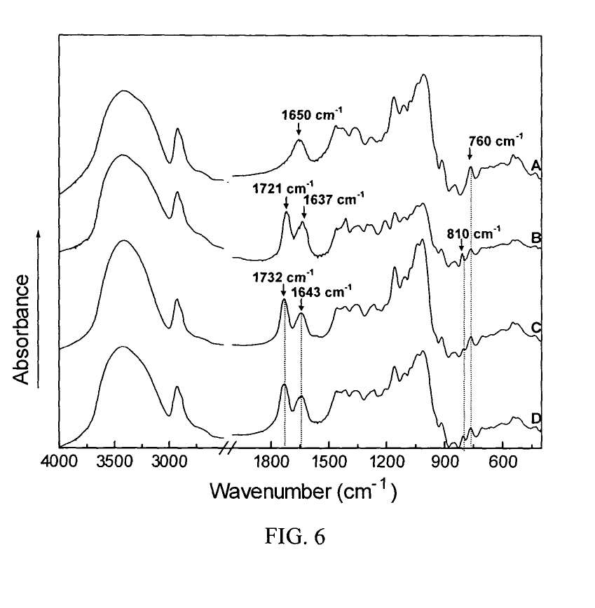

The hydrogels were further characterized by FTIR to assess the degree of polymerization (DP) of the acrylate groups. FIG. 6 shows the FTIR spectra of native dexT70, the dexT70-VA “monomer” (DS=31.5%), and the chemoenzymatically-generated hydrogels. In the dexT70-VA monomer (spectrum B) the absorption at 1721 and 810 cm⁻¹ are assigned to the stretching of the carbonyl group and to the twisting of the acrylate double bond, respectively. The absorption at 1637 cm⁻¹ (stretching) also indicates the presence of double bonds; however, it is overlaid with an absorption peak of the original dextran at 1650 cm⁻¹. The same absorption peaks were observed in the dexT70-VA gels (spectra C and D); however, the decrease in peak intensity at 810 cm⁻¹ indicates that solely some of the double bonds underwent polymerization. The relative decrease of the ratio of absorption at 810 cm⁻¹ and 760 cm⁻¹ (dextran) was used to calculate the conversion of the acrylate groups for the different gels. The results are presented in Table 3 (see DP values) and reveal that the conversion of the acrylate groups depends on the DS of dexT70-VA and its initial concentration (see below).

TABLE 3
Network properties of dexT70-VA gels as a function of the initial monomer
concentration and the degree of substitution.

| | W₀^{a} | | | | | \overline{M}_{c}^{e} | \overline{M}_{c}^{e} | | □ₓ^{g} |
|---|---|---|---|---|---|---|---|---|---|
| | (%, w/ | DS^{b} | DP | SRE^{d}, | SRE^{d}, | Eq. 2 | Eq. 3 | ξ^{f f} | (10⁻³) |
| Gel | v) | (%) | (%)^{c} | 25° C. | 37° C. | (g/mol) | (g/mol) | (A⁰) | (mol/cm³) |
| 1 | | 7.2 | 100.0 | 31.53 | —^{h} | 12389.1 | 12482.1 | 181.95 | 0.129 |
| 2 | | 15.1 | 100.0 | 14.36 | —^{h} | 4177.1 | 4464.0 | 84.74 | 0.361 |
| 3 | 8 | 22.4 | 100.0 | 11.01 | —^{h} | 2314.9 | 2668.3 | 60.36 | 0.605 |
| 4 | | 31.5 | 75.8 | 9.78 | —^{h} | 1714.6 | 2092.2 | 51.55 | 0.773 |
| 5 | | 37.0 | 54.8 | 9.41 | —^{h} | 1543.4 | 1928.0 | 48.91 | 0.837 |
| 6 | | 7.2 | —^{h} | 7.32 | 8.56 | 2248.6 | 2531.9 | 52.02 | 0.643 |
| 7 | | 15.1 | —^{h} | 4.39 | 4.99 | 659.6 | 1000.4 | 28.29 | 1.614 |
| 8 | 20 | 22.4 | —^{h} | 3.99 | 4.24 | 513.2 | 857.0 | 25.51 | 1.891 |
| 9 | | 31.5 | 28.7 | 3.72 | 3.88 | 424.2 | 769.0 | 23.73 | 2.098 |
| 10 | | 37.0 | 37.7 | 3.61 | 3.69 | 392.9 | 737.6 | 23.07 | 2.188 |
| 11 | | 7.2 | —^{h} | 5.62 | —^{h} | 1748.3 | 2021.7 | 43.05 | 0.824 |
| 12 | | 15.1 | —^{h} | 3.13 | —^{h} | 435.0 | 751.3 | 22.35 | 2.149 |
| 13 | 30 | 22.4 | —^{h} | 2.74 | —^{h} | 314.6 | 630.0 | 19.62 | 2.583 |
| 14 | | 31.5 | 26.1 | 2.58 | —^{h} | 269.5 | 584.2 | 18.87 | 2.762 |
| 15 | | 37.0 | 35.4 | 2.39 | —^{h} | 221.3 | 533.6 | 17.71 | 3.023 |

^{a}Initial monomer concentration.
^{b}Degree of substitution, i.e., the amount of vinyl groups per 100 fructose units.
^{c}Degree of polymerization obtained by FTIR using the A810/A760 ratio.
^{d}Swelling ratio at equilibrium.
^{e}Molecular weight between the crosslinks.
^{f}Average mesh size using \overline{M}_{c} from Eq. 3.
^{g}Crosslinking density.
^{h}Not calculated.

We proceeded to evaluate the structural properties of the crosslinked hydrogels. One important parameter is the average molecular weight of the polymer chain between two neighboring crosslinks (\overline{M}_{c}) [1]. These junctions may be chemical crosslinks, physical entanglements, crystalline regions, or even polymer complexes [1]. Parameters derived from \overline{M}_{c} include the crosslinking density (ρₓ) and the average mesh pore size (ξ). This latter parameter provides a measure of the space available between the macromolecular chains available for solute diffusion. \overline{M}_{c} was calculated by equilibrium swelling theory for the case of networks where the crosslinks were introduced in solution [22]. Eq. 2 applies to isotropic, loosely crosslinked networks, where the number of repeat units between crosslinks is large enough so that the chains can be represented by a Gaussian distribution (usually comprising 100 or more repeat units), while Eq. 3 applies to isotropic, highly crosslinked networks with a moderate degree of swelling. The \overline{M}_{c} values were calculated using these different theoretical analyses after the determination of the polymer volume fraction before (v₂,r) and after (v_{2,s}) swelling and the values are given in Table 3. The values of \overline{M}_{c} calculated from Eq. 2 are lower than those calculated from Eq. 3 for all gels, and this effect is more pronounced for dexT70-VA gels with high DS and obtained from higher initial monomer concentrations. The \overline{M}_{c} values are too small (fewer than 100 repeat units, considering that the molecular weight of each unit is 162.14 gmol-⁻¹) to assume a Gaussian distribution of the polymer chain lengths. Therefore Flory-Rehner analysis (Eq. 2) cannot be applied, and it must be replaced with Eq. 3, which takes into account deviations from the Gaussian distribution.

From \overline{M}_{c} values determined by Eq. 3, ξ was calculated (Table 3). SRE, \overline{M}_{c}, and ξ decrease as a function of monomer concentration from 8 to 30% (w/v), while maintaining a constant DS of dexT70-VA. The decrease of both parameters for higher dexT70-VA concentrations is likely due to an increase in the number of intermolecular crosslinks and physical entanglements formed (see below), which restricts network expansion upon swelling [9]. It should be mentioned that intramolecular crosslinks do not contribute to the elasticity of the network and therefore do not contribute for SRE, \overline{M}_{c}, and ξ [4]. Hence, gels obtained from lower initial monomer concentrations (gels 1-5) have a higher contribution of intramolecular linkages, whereas intermolecular crosslinks (and physical entanglements) are predominantly formed by more concentrated solutions (gels 11-15). However, keeping the same concentration of the initial monomer, the values of SRE, \overline{M}_{c} and ξ decrease as DS of the monomer increases in the polymerization solutions, which is mainly due to the increased number of intermolecular crosslinks favored by the high number of acrylate groups attached to dextran.

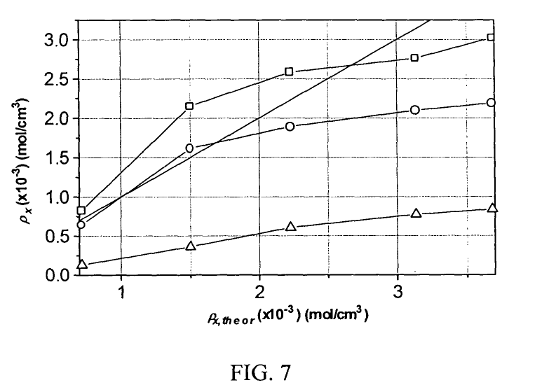

The aforementioned explanations were confirmed by comparing the results of FIG. 7 and the results of degree of polymerization (DP) obtained by FTIR presented in Table 3. ρₓ values for gels 1-5 is lower than the ρ_{x,theor} which confirms mainly the formation of intramolecular crosslinks. Not surprisingly, as the DS of dexT70-VA increases contributions from intermolecular crosslinks increase slightly as shown by the increase in ρₓ (FIG. 7). The DP of gels 1-3 was 100% (total polymerization of the acrylate groups), while for gels 4 and 5, the DP was 75.8 and 54.8%, respectively, indicating the existence of unreacted acrylate groups. These results show that gels obtained from dexT70-VA with high DS present a higher contribution from intermolecular crosslinks than gels with low DS despite the fact that some of the acrylate groups did not react.

The properties of gels 11-15 are clearly different from gels 1-5. ρₓ values are higher than ρ_{x,theor} for gels 11-13 (FIG. 7) but lower for gels 14-15. The results indicate that gels 11-13 present intermolecular crosslinks and physical entanglements that increase ρₓ relative to the expected value calculated by ρ_{x,theor} (which does not take into account the effect of physical entanglements). For gels 14-15 one would expect the same contribution of the intermolecular crosslinks and physical entanglements; however ρₓ is lower than ρ_{x,theor}. This is likely due to the low degree of polymerization (<40%) of the acrylate groups (Table 3).

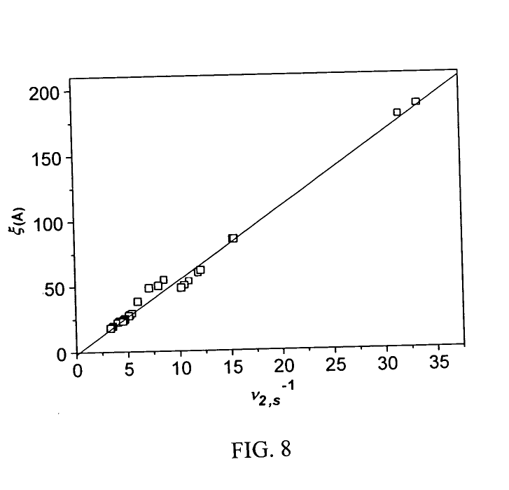

A major proposed application of dexT70-VA gels is as drug delivery carriers. The determination of ξ serves as a useful measure of the nature of the network on drug diffusion [33]. Establishment of a correlation between ξ and v_{2,s} will make it easier to predict which drug may be loaded in the gel by the simple determination of the swelling characteristics of the gel. According to the literature, the correlation between v_{2,s} and ξ depends on the polymer concentration and its physicochemical properties [33]. For all dexT70-VA gels prepared (see Table 3) a linear regression with a predetermined exponent was fit (see Eq. 7). The best fit (FIG. 8) showed that ξ is related to v_{2,s} through a power of −1(r²=0.9899, k₁=−1.56 and k₂=5.56):

ξ=k₁+k₂v_{2,s}ⁿ (7)

A similar power law fit was reported by Canal & Peppas [33] for PVA gels. In theory, this correlation can be used to determine the ξ of dexT70-VA hydrogels with different crosslinking densities by only a single determination of the swelling of the hydrogel and, therefore, to predict the influence of the hydrogel on drug diffusion.

### REFERENCES FOR EXAMPLE 1

[1] Peppas NA, Bures P, Leobandung W, Ichikawa H. Hydrogels in pharmaceutical formulations. Eur J Pharm Biopharm 2000;50:27-46.

[2] Murphy SM, Hamilton CJ, Tighe BJ. Synthetic hydrogels:5. Transport processes in 2-hydroxyethyl methacrylate copolymers. Polymer 1988;29:1887-1893.

[3] Draye J-PD, Delaey B, van de Voorde A, van den Bulcke A, de Reu B, Schacht E. In vitro and in vivo biocompatibility of dextran dialdehyde cross-linked gelatin hydrogel films. Biomaterials 1998; 19:1677-1687.

[4] Hennink WE, Talsma H, Borchert JCH, de Smedt SC, Demeester J. Controlled release of proteins from dextran hydrogels. J Control Release 1996;39:47-55.

[5] Patil NS, Dordick JS, Rethwisch DG. Macroporous poly(sucrose acrylate) hydrogel for controlled release of macromolecules. Biomaterials 1996:17:2343-2350.

[6] Novick SJ, Dordick JS. Preparation of active and stable biocatalytic hydrogels for use in selective transformations. Chem Mater 1998;10:955-958.

[7] Jen AC, Wake MC, Mikos AG. Review: Hydrogels for cell immobilization. Biotechnol Bioeng 1996;50:357-364.

[8] Brondsted H, Hovgaard L, Simonsen L. Dextran hydrogels for colon-specific drug delivery II. Synthesis and characterization. Eur J Pharm Biopharm 1996;42(1):85-89.

[9] van Dijk-Wolthuis WNE, Franssen 0, Talsma H, van Steenbergen MJ, Kettenes-van den Bosch JJ, Hennink WE. Synthesis, characterization, and polymerization of glycidyl methacrylate derivatized dextran. Macromolecules 1995;28:6317-6322.

[10] Kim S-H, Chu C-C. Synthesis and characterization of dextran-methacrylate hydrogels and structural study by SEM. J Biomed Mater Res 2000;49:517-527.

[11] Zhang Y, Won C-Y, Chu C-C. Synthesis and characterization of biodegradable network hydrogels having both hydrophobic and hydrophilic components with controlled swelling behavior. J Polym Sci A: Polym Chem 1999;37: 4554-4569.

[12] Kim S-H, Won C-Y, Chu C-C. Synthesis and characterization of dextran-maleic acid based hydrogel. J Biomed Mater Res 1999;46:160-170.

[13] van Dijk-Wolthuis WNE, Kettenes-van den Bosch JJ, van der Kerk-van Hoof A, Hennink WE. Reaction of dextran with glycidyl methacrylate: an unexpected transesterification. Macromolecules 1997;30:3411-3413.

[14] Martin BD, Linhardt RJ, Dordick JS. Highly swelling hydrogels from ordered galactose-based polyacrylates. Biomaterials 1998; 19:69-76.

[15] Zaks A, Klibanov AM. Enzymatic catalysis in nonaqueous solvents. J Biol Chem 1988;263(7):3194-3201.

[16] Kwon OH, Imanishi Y, Ito Y. Catalytic activity and conformation of chemically modified subtilisin Carlsberg in organic media. Biotechnol Bioeng 1999;66(4):265-270.

[17] Hovgaard L, Brøndsted H. Dextran hydrogels for colon-specific drug delivery. J Control Release 1995;36:159-166.

[18] Vervoort L, van den Mooter G, Augustijns P, Busson R, Toppet S, Kinget R. Inulin hydrogels as carriers for colonic drug targeting: I. Synthesis and characterization of methacrylated inulin and hydrogel formation. Pharm Res 1997;14(12):1730-1737.

[19] Klibanov AM. Enzymes that work in organic solvents. CHEMTECH 1986; 16:354-359.

[20] Ferreira, L. MSc dissertation. Coimbra University, Portugal., 1997.

[21] Flory PJ, Rehner R. Statistical mechanics of cross-linked polymer networks. II. Swelling. J Chem Phys 1943;11(11):521-526.

[22] Peppas NA, Moynihan HJ, Lucht LM. The structure of highly crosslinked poly(2-hydroxyethyl methacrylate) hydrogels. J Biomed Mater Res 1985;19:397-411.

[23] van Dijk-Wolthuis WNE, Hoogeboom JAM, an Steenbergen MJ, Tsang SKY, Hennink WE. Degradation and release behavior of dextran-based hydrogels. Macromolecules 1997;30:4639-4645.

[24] Gekko K. Solution properties of dextran and its ionic derivatives. In ACS Symposium series vol. 150; Brant, D.A., Ed., American Chemical Society: Washington, D.C., 1981, 415-438.

[25] Almarsson Ö, Klibanov AM. Remarkable activation of enzymes in nonaqueous media by denaturing organic cosolvents. Biotechnol Bioeng 1996;49:87-92.

[26] Jackson M, Mantsch HH. Beware of proteins in DMSO. Biochim Biophys Acta 1991;1078:231-235.

[27] Chin JT, Wheeler SL, Klibanov AM. On protein solubility in organic solvents. Biotechnol Bioeng 1994;44:140-145.

[28] Otamiri M, Adlercreutz P, Mattiasson B. Effect on ester synthesis in toluene by immobilized chymotrypsin by addition of polymers to reaction medium. Biotechnol Appl Biochem 1991;13:54-64.

[29] Yoshimoto K, Itatani Y, Tsuda Y. ¹³C-Nuclear Magnetic Resonance (NMR) spectra of O-acylglucoses. Additivity of shift parameters and is application to structure elucidations. Chem Pharm Bull 1980;28(7):2065-2076.

[30] Therisod M, Klibanov AM. Regioselective acylation of secondary hydroxyl groups in sugars catalyzed by lipases in organic solvents. J Am Chem Soc 1987;109:3977-3981.

[31] MacManus DA, Vulfson EN. Substituent effects on the regioselectivity of enzymatic acylation of 6-O-alkylglycopyranosides using Pseudomonas cepacia lipase. Carbohydr Res 1995;279:281-291.

[32] Rich JO, Bedell BA, Dordick JS. Controlling enzyme-catalyzed regioselectivity in sugar ester synthesis. Biotechnol Bioeng 1995;45:426-434.

[33] Canal T, Peppas NA. Correlation between mesh size and equilibrium degree of swelling of polymeric networks. J Biomed Mater Res 1989;23:1183-1193.

[34] Chaudhary, A. K.; Beckman, E. J.; Russell, A. J. Biotechnol Bioeng 1997, 55, 227. Brazwell, E. M.; Filos, D. Y.; Morrow, C. J. J Polym Sci Part A: Polym Chem 1995, 33, 89.

### Example 2

Biocatalytic Synthesis of Biocompatible and Degradable Dextran-Based Hydrogels

Overview

This example describes a biocatalytic strategy to obtain unique macropourous and ordered dextran-based hydrogels, which involves a single step transesterification reaction between dextran (Mw>6 kDa) and divinylapidate (DVA) in neat DMSO. The resulting hydrogenls were degradable at physiologic pHs, biocompatible (without substation fibrous capsule formation), sustained protein release for several days and had superior mechanical properties as compared to dextran-based hydrogels obtained chemically from similar starting materials.

The site-selective proliferation of cells and the modulation of cellular function at implantable sites requires the controlled release of biologically active macromolecules, including growth factors¹⁻³, and plasmid DNA⁴. These molecules must be incorporated into biocompatible, and ultimately erodable/biodegradable, matrices that provide for useful materials properties while maintaining biologically-relevant functionality, including potential use as scaffolds to deliver cells to specific anatomic sites, create and maintain a space for tissue development, and guide tissue formation before being degraded⁵. This has served as the driving force for the synthesis of a wide range of natural and synthetic polymers with biocompatible and erodable/biodegradable properties⁶⁻⁷, including aliphatic polyesters, such as poly(glycolic acid) (PGA) and poly(lactic acid) (PLA) and their copolymers, polyanhydrides, among others. However PGA and PLA are stiff materials that make them unsuitable as matrices for soft tissue engineering⁶. In addition, due to the release of lactic or glycolic acid⁸, or the hydrolysis of anhydrides, the resulting drop in pH often results in deactivation of proteins through unfolding and aggregation². Alternative natural polymers have been used, for example bovine collagen, which has potential risks for disease transmission (e.g., spongiform encephalopathies)⁹ and undesirable immune response, and polysaccharides. The latter may be an alternative to the aforementioned polymers especially for soft tissue engineering applications.

Dextrans, in particular, are ideal natural products for biomedical applications¹⁰. These glucose-based polymers are available in a wide range of sizes and contain a high density of hydroxyl groups that makes the polymer highly hydrophilic and capable of being further functionalized chemically or enzymatically. Finally, dextran is biocompatible and can be degraded in the human digestive system through the action of dextranases. Native dextrans are not hydrogels; however, crosslinking with various agents has resulted in the formation of dextran-based hydrogels¹¹⁻¹⁴. Unfortunately, these chemical routes lack sufficient regioselectivity, thereby resulting in irregular 3D orientation and large pore size distributions¹⁵, which can result in dense regions that are unable to release entrapped bioactive molecules¹⁶, and ultimately in poor mechanical properties. Furthermore the heterogeneity of the hydrogel structure may limit biological performance¹⁷.

Materials and Methods

Methods

Enzyme Screening for the Transesterification reaction of dexT70 with DVA

The enzymes were “pH-adjusted” prior to use in the presence of 20 mM phosphate buffer at pH 8.0 (“Proleather”, Protease S, and subtilisin Carlsberg) or at pH 7.5 (Proteases A, N, and P, and Lipases A, AY, M, PS, and Porcine Pancreas). After being flash-frozen in liquid nitrogen, the samples were lyophilized on a Labconco freeze-drier (Labconco Corp., Kansas City, Mo.) for 48 h. Proleather thermally deactivated or inhibited by PMSF were prepared as previously²⁰. Reactions were performed in 15 mL of anhydrous DMSO (0.06% of water content as measured by Karl-Fischer titration) containing dextran M_{w} 70 kDa (1 g; Fluka Chemie AG, Buchs, Switzerland), 0.204 M DVA (TCI America, Portland, Oreg.) and 300 mg of “pH-adjusted” enzymes (except for subtilisin Carlsberg, which was employed at 150 mg). The reaction mixtures were shaken at 50° C. and 250 rpm for 72 h (except for Proleather FG-F and lipases AY and PS, which was 48 h). The purification of the products was performed as below.

Proleather FG-F Catalyzed Synthesis of dexT110-DVA, dexT70-DVA and dexT40-DVA

Dextran (1 g) (dexT40: M_{w}=40 kDa; dexT70: M_{w}=70 kDa; and dexT 110:M_{w}=110 kDa, according to the manufacturer's specification; Fluka Chemie AG, Buchs, Switzerland) and a calculated amount of DVA (0.123-1.224 g) were dissolved in DMSO (15 mL) and the reaction initiated by adding 300 mg of “pH-adjusted” Proleather. The reaction mixtures were shaken at 50° C. (250 rpm) for 72 h. In the reaction mixtures that did not gel, the solutions were centrifuged at 4,000 rpm for 10 min and the supernatants mixed with water (3:7, v/v) and dialyzed (MWCO of 50,000 or 1,000; Spectrum, Calif.) against HCl aqueous solution pH 3.0 for 7 days and 3 more days against milli-Q water, at 4° C. Afterwards the aqueous solutions were freeze-dried for 48 h (isolated yields between 60 and 85%). In reaction mixtures that did gel, the resulting gels were separated from the glass beakers and immersed in milli-Q water, at 4° C., for 10 days, changing the water daily. Afterwards, the gels were cut in disc-shape sections with a thickness of 0.4-0.6 cm, dried at room temperature, under vacuum in the presence of phosphorous pentoxide until constant weight (isolated yields between 72 and 87%). The DS of dextran derivatives (either in the gel or non-gel form) was determined by FTIR in a spectrometer Nicolet Magna-IR 550 using KBr pellets, taking into account the ratio of the absorption bands at 1730 cm⁻¹ (ester, v (C═O)) and 760 cm⁻¹ (dextran). The ratio (A₁₇₃₀/A₇₆₀) values were then converted into DS using a calibration curve of dextran M_{w} 6 kDa derivatized with different concentrations of DVA (water-soluble compounds), where DS was calculated using ¹H NMR spectroscopy. In this case, DS=[(7×x)/(4×y)]×100, where x is the integral of the adipate protons in the range of δ2.47−1.64 ppm and y is the integral of all dextran protons between δ5.56−3.20 ppm.

Proleather Transesterification Reaction of Sugars or Inulin with DVA

Reactions were performed in 5 mL of anhydrous solvents containing 0.1 M of sugar, 0.2 M DVA and 75 mg of “pH-adjusted” Proleather FG-F. The reaction mixtures were shaken at 50° C. and 250 rpm. Periodically, 100 μL aliquots were removed, centrifuged at 4000 rpm for 5 min, and the supernatant analyzed by Gas Chromatography³⁹. The extent of the enzymatic acylation was calculated from the decrease in the concentration of the sugar substrate. In case of inulin reactions, they were performed in 15 mL of anhydrous DMSO containing 17 mM inulin (M_{w}=4.2 kDa, Fluka Chemie AG, Buchs, Switzerland), 200 mM DVA, and 300 mg of “pH-adjusted” Proleather FG-F. The reaction mixtures were shaken at 50° C. (250 rpm) in orbital shaker for 140 h, after which they were centrifuged at 4,000 rpm for 10 min. The supernatants were mixed with water (3:7, v/v) and dialyzed using a regenerated cellulose dialysis tube with a 1,000 MWCO for 2 days, at 4° C., against milli-Q water. Afterward the aqueous solutions were lyophilized for 48 h. The conversion and isolated yield were 59 and 62%, respectively.

Chemical Synthesis of dexT70-DVA

Dextran (dexT70, 1 g) and a calculated amount of DVA (0.123-1.224 g) were dissolved in DMSO (15 mL) and the reaction commenced by adding 4-DMAP (200 mg). The reaction mixtures were shaken at 50° C. (250 rpm) for 72 h. In the reaction mixtures that gel, the resulting gels were separated from the glass beakers and immersed in milli-Q water adjusted to pH 3.0, for 10 days at 4° C., changing the water daily. Afterwards, the hydrogels were dried at room temperature, under vacuum, until constant weight.

Scanning Electron Microscopy

Swollen dexT70-DVA hydrogels were quickly frozen in a glass container using liquid nitrogen and freeze-dried for 48 h. Fractured pieces of 0.6-0.9 cm in length, corresponding to half-diameter of each hydrogel, were mounted into aluminium stud and gold coated by plasma vapor deposition. The surface and cross-section of hydrogels were recorded by a field emission scanning electron microscope (JEOL model JSM-5310), at 15.0-20.0 kV. Analyses of the digitized images were performed using Scion Image (Scion Corporation, Maryland), according to a methodology described elsewhere¹⁵. The diameter of a pore was obtained by averaging the major and minor axes of the pore.

Mercury Intrusion Porosimetry

Mercury porosimetry (Micromeritics Poresizer 9320) was used to determine the bulk density, skeletal density, porosity and pore size distribution. All the samples were degassed before analysis at a vacuum pressure below 50 mm Hg. High pressure runs (from 25 up to 30,000 psia) were made with an equilibration time of 20 s and a maximum intrusion volume of 0.0500 mL/g. The porograms (intruded volume versus pressure) obtained were converted into pore diameter distribution curves (cumulative and differential) according to the Washburn⁴⁰ equation, pd=−4r cos θ, where p is the pressure required to force mercury into a pore of entry diameter d, r is the surface tension (485 dyncm⁻¹), and θ is the contact angle between mercury and the sample (130°). The porosity of hydrogels was calculated from the equation: porosity=((skeletal density-bulk density)/skeletal density)×100.

Protein Release Studies

Bovine serum albumin and lysozyme from chicken egg white (Sigma, St. Louis, Mo.) were loaded onto dexT70-DVA DS 31% hydrogel by absorption from solution. The hydrogels were incubated with 6 mL of protein solution (either 1.25% or 5% (w/v)) for 5 days, at 25° C. Afterwards the hydrogels were rinsed twice with 10 mL of buffer solution (pH 7.4 or 5.0) and finally incubated in 4 mL of the same buffer (with 0.02% of sodium azide), at 37° C., under agitation (100 rpm). The incubation medium was changed several times to maintain negligible solution concentration. The BCA assay (Pierce, Rockford, Ill.) was used to determine the concentration of protein released from hydrogels. The absorbance was measured at 540 nm using a 96-well plate spectrophotometer (STL Spectra III, Austria). To determine the remaining protein content in the interior of the hydrogel after release, the hydrogels were powdered and incubated in 10 mM PBS (adjusted to pH 9-O-9.5 with 0.1 M NaOH) upon complete degradation. In case of hydrogels used for the uptake and release at pH 5.0, they were incubated in TCA 5%, at 37° C., upon complete degradation (ca. 48 h). The solutions were then freeze-dried for 24 h and resuspended in 4 mL of PBSTEU (phosphate buffer saline with 0.02% of Tween 80, 1 mM of EDTA and 6 M of urea) for 2-3 h, at 37° C. Using PBSTEU, any noncovalently bound BSA aggregates will be dissolved². The protein concentration was determined after using protein calibration curves in the same conditions as used for each sample. The enzymatic activity of lysozyme was determined using Micrococcus lysodeikticus bacterial cells as a substrate⁴¹.

In vivo Biocompatibility Studies

European community guidelines (n° 86/609/CE; corresponding to decree n° 1005/92 of Portuguese legislation) for the care and use of laboratory animals were observed. Male Wistar rats (eight to twelve-week-old) were anesthetized, with Ketalar® (50 mgKg⁻¹, Parke-Davis) and an area of the front was shaved and washed with Betadine®. Two incisions along the spine (ca. 1 cm in length) were made and two subcutaneous pockets were created. The hydrogels (3 mm×2 mm, 3 mm thickness), previously UV-sterilized and swollen in citrate-phosphate pH 5.0, were placed into the pocket away from the incision (ca. 0.5 cm) and the skin was closed with Mersilk® non-absorbable suture (3 Øs, Ethicon). The area was washed with Betadine® dermic solution and the rats kept warm with a heating pad for 1 h after the surgical procedure and finally transferred to a cage. Three rats were used for each time point. As control, subcutaneous pockets were made without implants. At certain times, the rats were sacrificed and the implants with surrounding tissue where carefully dissected and fixed in 4% (v/v) neutral buffered formalin, for at least 3 days. The blocks were sliced perpendicular or cross-sectional to the implanted hydrogels. The implant and adjacent tissue were oriented and placed in processing cassettes, taken through a graded ethanol series (Shandon Citadel 1000) and embebbed in paraffin. The samples were then sectioned using a microtome (Shandon Retraction AS 325), and finally deparaffinized and stained either with hematoxylin/eosin, periodic acid-schiff or Masson's trichrome ⁴².

Results and Discussion

Biocatalytic Synthesis Of Dextran-based Hydrogels

Dextran is soluble in water or very polar organic solvents such as DMSO; however, only in the latter can enzyme-catalyzed transesterification of dextran be performed without undesirable ester hydrolysis. To that end, we proceeded to identify enzymes with biocatalytic activity in DMSO. Eleven enzymes, chosen from a group of hydrolases that are known to catalyze the acylation of simple sugars or other polysaccharides in organic media, were screened for their abilities to catalyze the acylation of dextran (m.w. 70,000 (dexT70)) with DVA (Table 4).

TABLE 4
Enzyme screening for the transesterification reaction of dexT70 with DVA.

| | | | Reaction | |
|---|---|---|---|---|
| Entry | Enzyme | Enzyme source | time (h) | Conversion (%)^{c} |
| 1 | Proleather FG-F^{a} | Bacillus subtilis | 48 | 71.4 (gel)^{d} |
| 2 | Protease A^{a} | Aspergillus oryzae | 72 | 4.2^{e} |
| 3 | Protease N^{a} | Bacillus subtilis | 72 | 4.3^{e} |
| 4 | Protease P^{a} | Aspergillus melleus | 72 | 8.2^{e} |
| 5 | Protease S^{a} | Bacillus stearothermophilus | 72 | <1.0^{e} |
| 6 | Subtilisin Carlsberg^{b} | Bacillus licheniformis | 72 | 7.2^{e} |
| 7 | Lipase A^{a} | Aspergillus niger | 72 | 7.1^{e} |
| 8 | Lipase AY^{a} | Candida rugosa | 48 | 62.6 (gel)^{d} |
| 9 | Lipase M^{a} | Mucor javanicus | 72 | 25.0^{e} |
| 10 | Lipase PS^{a} | Pseudomonas cepacia | 48 | 58.4 (gel)^{d} |
| 11 | Lipase Porcine | Porcine Pancreas | 72 | 3.0^{e} |
| | Pancreas, type II^{b} | | | |

^{a}Obtained from Amano Enzyme Co. (Troy, VA).
^{b}Obtained from Sigma (St. Louis, MO).
^{c}The conversion is defined as the percentage of DVA molecules incorporated into dextran through single or double ester bonds taking into account the initial molar ratio of DVA to dextran glucopyranose residues in the reaction mixture.
^{d}Determined by Fourier Transform Infrared using KBr pellets.
^{e}Determined by back titration with 0.1 N HCl using hpenolphthalein as indicator

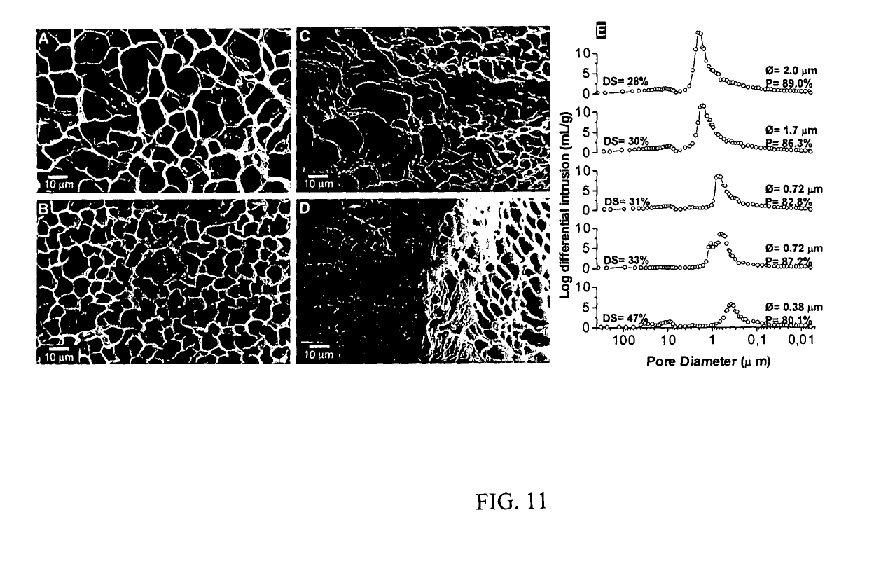

Three of the enzymes gave appreciable preliminary calculated conversion, with an alkaline protease from B. subtilis (Proleather FG-F) and two lipases (from C. rugosa and P. cepacia) yielding>58% conversion of the dextran. These reactions resulted in the formation of a gel, which did not form in control reactions in the absence of enzyme or in the presence of the aforementioned enzymes in the absence of DVA. FTIR spectra (FIG. 10) of the gels confirmed the presence of carbonyl groups (peak at 1730 cm⁻¹) arising from the DVA molecules attached to dextran. To complement these results, CP/MAS ¹³C NMR spectroscopy was undertaken (FIG. 11), and provided evidence of the carbonyl carbon at δ175.8 ppm and the adipate carbons at δ32.5 and 25.9 ppm further confirming the transesterification of dextran with DVA.

Interestingly, all three enzymes were soluble in the organic solvent, which was surprising given the well-known deactivation of enzymes in neat DMSO¹⁸⁻¹⁹. To confirm that the transesterification reaction was indeed enzymatic, several control reactions were performed using Proleather as the most reactive enzyme. Limited spontaneous reaction (conversion<15% with no gel formation) was observed in the absence of enzyme or with thermally-deactivated Proleather over a period of 72 h (FIG. 10A). Enzyme pre-inactivated by the serine protease inhibitor phenylmethanesulfonyl fluoride (PMSF) also gave minimal reactivity and again no gel formation was observed. The enzyme activity was not believed to be due to an underlying contaminant in the commercial preparation. The ability of Proleather to remain active while dissolved in DMSO was strongly dependent on the nature of the enzyme preparation. The commercial “Proleather” preparation contains only ca. 13% (w/w) protein. Upon dialysis to increase the protein content to 40% (w/w) the dextran conversion was reduced two-fold compared to the crude enzyme preparation. Proleather was also active in DMSO on simple compounds related to dextran, including the simple sugar O-methyl-β-D-glucoside. Interestingly, as the sugar size is increased to maltose and maltotriose, Proleather activity in DMSO increased (Table 5).

TABLE 5
Activity of Proleather FG-F in the transesterification
reaction of different sugars with DVA.

| | | Specific Activity | |
|---|---|---|---|
| | | (μmol/mg active | Conversion at 12 h |
| Nucleophile | Solvent | enzyme* min)^{a} | (%) |
| Sucrose | DMF | 3.24 | 100.0 |
| Maltose | DMF | 1.18 | 98.1 |
| α-Methyl glucose | DMSO | 0.52 | 17.3 |
| Sucrose | DMSO | 9.84 | 86.6 |
| Maltose | DMSO | 1.39 | 64.4 |
| Trehalose | DMSO | 1.06 | 23.1 |
| Raffinose | DMSO | 8.41 | 91.6 |
| Maltotriose | DMSO | 11.15 | 100.0 |

^{a}The total active enzyme was 2.89 ± 0.56% (w/w), as determined by N-trancinnamoylimidazole active sites titration in aqueous buffer (Ref. 26).

These results suggest that the presence of sugars of increasing size (including the polysaccharide dextran) help stabilize Proleather in the soluble state in DMSO and enable transesterification reactions to be performed.

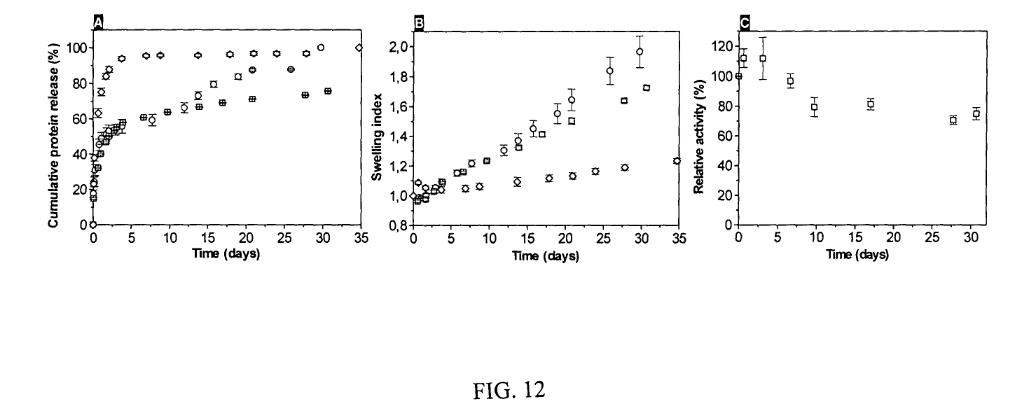

The time course of Proleather-catalyzed dextran acylation is depicted in FIG. 10A, where a rapid increase is observed in the degree of substitution (DS; defined as the number of adipate groups incorporated into dextran per 100 dextran glucopyranoside residues). This is concomitant with a decrease in the equilibrium swelling ratio of the hydrogels (FIG. 10B), which continues even past the point of an observable increase in the extent of acylation, and a substantial decrease in average pore diameter (FIGS. 10C and D) from 20.9±7.6 μm (average±SD, n=74) to 6.3+2.8 μm (n=159) along with an increase in pore wall thickness. This is likely due to the rapid acylation of dextran with DVA followed by a slower intra- and/or intermolecular reaction of the second vinyl ester group of DVA with another part of the dextran chain or other dextran chains to form adipate crosslinks. To distinguish between these two types of crosslinking, we examined the influence of dextran macromonomer molecular weight on the swelling behavior of the resulting hydrogels. Lower swelling is obtained with higher molecular weight dextran macromonomers; at 27.5% DS, the SR is ca. 20 for 110 kDa dextran and this increases to 28 for 70 kDa and 47 for 40 kDa dextran, respectively (FIG. 12 of Supplemental Material). Because it is unlikely that intramolecular crosslinks affect SR, as they will not affect polymer size, the dependence of SR on macromonomer size strongly suggests that intermolecular macrochain formation predominated over intramolecular crosslinking.

Physicochemical Properties of Dextran-DVA Hydrogels

The dextran-based gels were viscoelastic, with moduli ranging from 1.4 kPa for gels prepared with a DS of 20 to 5.8 kPa for gels prepared with a DS of 45, as measured by an indentation method²¹. Interestingly, the hydrogels prepared biocatalytically show a higher elastic modulus for a given swelling ratio than similar hydrogels synthesized chemically. For example, for hydrogels with calculated polymer volume fractions of 0.04-0.05, the elastic modulus of dexT70 biococatalytic hydrogels is twice as high of a gel obtained via chemical crosslinking with hexamethylenediisocyanate¹¹ (4.9 vs 2.2 kPa) and 4-fold higher than dexT40-methacrylate hydrogels²³ (2.6 vs 0.6 kPa). For a more direct comparison, dextran was acylated chemically with DVA using 4-dimethylaminopyridine (4-DMAP) as catalyst in DMSO at 50° C.¹². Hydrogel formation required 40% DS, in contrast to 20% needed for gel formation in the enzymatic process. Even at 40% DS, the chemically-generated hydrogels were fragile and easily fragmented preventing modulus determination. These results suggest that Proleather catalysis favors the formation of a greater number of intermolecular crosslinks as compared to the chemical route. Similar results have been described for the polytransesterification reaction of inulin with DVA, where transesterification of the inulin macromonomer resulted in an increase in the molecular weight of the hydrogel product²⁴.

Differences between the enzymatically and chemically (4-DMAP catalyzed) synthesized dexT70 hydrogels was further highlighted using SEM analysis of inner regions of the gels (FIG. 11). The biocatalytic dextran hydrogels can be characterized as having larger and more uniform pore sizes than those of chemically prepared hydrogels. The enhanced structural organization of biocatalytic hydrogels was also confirmed by mercury intrusion porosimetry (MIP) analysis (FIG. 11E). Hydrogels showed a unimodal distribution of pores with average diameters from 0.4-2.0 μm. The sharp peaks of the porograms show that the pore size distribution is narrow and relatively homogeneous. Finally, the porosity values were higher than 80% and show that hydrogels are formed by an interconnected structure. This finding is particularly relevant for the use of these hydrogels in tissue engineering field since porosities above 80% are desired for tissue integration²⁵.

The structural organization of biocatalytic dexT70-DVA hydrogels is also distinct to other dextran-based hydrogels prepared chemically. Chu and co-workers¹⁵ have reported differences in the pore size distribution between the surface and interior of dextran-methacrylate hydrogels. In our case, smaller pores were observed closer to the surface where most of the crosslinking occurred, while larger pores were observed closer to the core of the material. In addition, SEM and MIP showed a bimodal distribution of pore sizes either in mesoporous or macroporous regions. The homogeneity in dexT70-DVA hydrogels obtained enzymatically may be at least partly due to the regioselectivity achieved in the enzymatic process and the uniform crosslinking of the dextran promoted by a homogeneous distribution of the biological catalyst. Furthermore, we observed that hydrogel history had an important effect in its ultimate structural organization. For example, a drying step after hydrogel preparation enhances its structural organization²⁶.

Dextran-based Hydrogels as Protein Controlled-release Systems

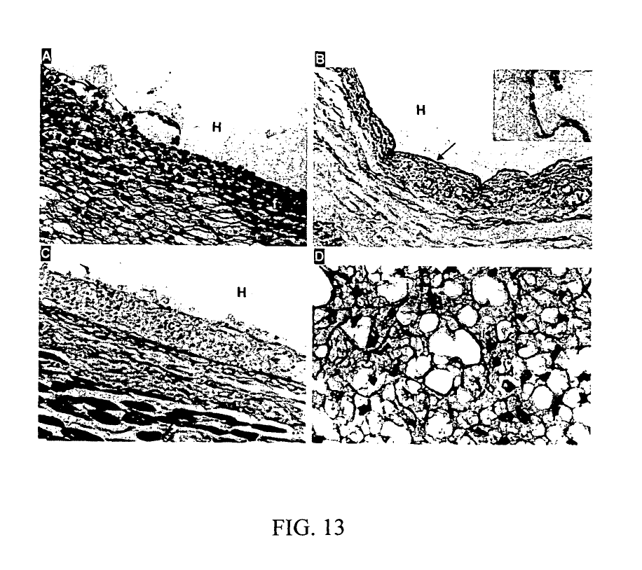

The macroporous structure of dexT70-DVA hydrogels may serve as ideal matrices for controlled release of proteins. To that end, the uptake and release of two model proteins, lysozyme and bovine serum albumin (BSA), were performed with dexT70-DVA DS 31% hydrogels, in phosphate buffer saline (PBS) pH 7.4. Lysozyme uptake was nearly twice as high as expected theoretically (taking into account the swelling of the hydrogel and the protein feed concentration) (Table 4, Supplemental material). The higher than theoretical protein loading may be due to electrostatic attraction between the hydrogel matrix and lysozyme that occurs during initial stages of hydrogel degradation. During degradation of Dext70-DVA hydrogels in PBS (FIG. 13 of Supplemental Material) adipate molecules singly attached to dextran are formed, carrying terminal carboxylate groups. At this pH, lysozyme is cationic and favors direct electrostatic interaction with the newly formed carboxylate groups in dexT70-DVA hydrogels, thereby increasing the partitioning of lysozyme into the hydrogel network. Conversely, BSA uptake was less than 10% of the theoretical level, and this was ascribed to the anionic characteri of BSA at pH 7.4.

FIG. 12A displays the release profile of lysozyme (14 kDa) and BSA (65 kDa) in 10 mM PBS pH 7.4, at 37° C., from dexT70-DVA DS 31% hydrogels. An initial burst release over the first 2 days was followed by a longer period (<30 days) of sustained release. Without being bound to any theory, two major factors may contribute to the protein release. First, the electrostatic desorption of the protein from the network. Indeed, when the electrostatic interactions between BSA and the hydrogel were prevented at pH 5.0 (at this pH the hydrogel degradation is nearly absent (FIG. 12B) and the net charge of BSA is ca. null), 90% of total BSA uptake by the hydrogel was released during the first 4 days (FIG. 12A). It should be noted that the sustained release is longer for lysozyme than BSA which is likely due to the net positive charge (+7)²⁹ which favors a stronger interaction with the carboxylate groups of hydrogel. Second, as hydrogel degrades, its swelling (FIG. 3B) and size increases and the concentration of the protein in the interior decreases. Both effects result in a decrease in protein concentration gradient, and therefore in a decreasing release rate.

These results show that it is possible to sustain protein release even with macroporous hydrogels which usually yield a faster protein release profile³⁰⁻³¹. Furthermore, the sustained release of dexT70-DVA hydrogels is achieved during the degradation of the network, involving electrostatic interactions, which is quite singular. A part quantitative release of proteins, preservation of its structural integrity and thus biological activity is a main requisite. The specific activity of lysozyme after being released from hydrogels only decreased ca. 25% from its initial activity (FIG. 12C), which shows that protein-hydrogel interaction did not affect significantly the biological activity of lysozyme.

Biocompatibility of dexT70-D VA Hydrogels

The biocompatibility of the dextran-based hydrogels were assessed via subcutaneous and intramuscularly (data not shown) implantation using gels of DS values ranging from 28-47%. The intensity of the inflammatory response to these foreign implants was monitored histologically and representative light micrographs of dexT70-DVA DS 31% hydrogel implanted subcutaneously are presented in FIG. 13. In general, the dextran-based hydrogels showed good in vivo biocompatibility. The 10-day wound healing response to the implants consisted initially of macrophages (foam cells) eroding the dextran hydrogel surface, fibroblasts depositing collagen into the region of the implant, and the presence of few foreign body giant (FBG) cells formed by fusion of macrophages, particularly in high DS dextran-based hydrogels. These tissue responses are consistent with those observed in other biocompatible hydrogel implants³²⁻³³. Specifically, the influence of DS was clearly evident in subcutaneous implantation. Foam cells were observed at 5, 10 and 40 days postimplantation for hydrogels with DS values of 28, 31, and 47%, respectively. These cells contained hydrogel particles, as confirmed by periodic acid-schiff staining, which suggests a phagocytosis process. As expected, this phenomenon starts earlier for hydrogels with lower DS, primarily because of the smaller number of crosslinks and the easier degradability. FBG cells were observed mainly in hydrogel DS 47%. The high number of FBG cells in this last network is likely due to its rough surface 32. Between the foam and FBG cells, a layer of collagen-depositing fibroblasts were observed. This collagen was neither continuous nor dense and, therefore, did not form a truly fibrous capsule around the hydrogel. The lack of fully intact fibrous capsule formation is relatively unique in comparison to other non-degradable³⁴ and degradable^{8,35} hydrogels, which favor the formation of fibrous capsule when implanted in vivo. The incomplete fibrous capsule formation in the dextran-based gels is critical in supporting ultimate gel integration within the surrounding tissue. Despite the lack of a fully intact fibrous capsule, the fibroblasts were able to adhere to hydrogels. While dextran has been shown to be a low-protein binding polysaccharide³⁶⁻³⁷ and a cell resistant material³⁸, the adhesion of fibroblasts in the dexT70-DVA hydrogels may be due to the charged adipic acid residues present on the hydrogel surface. Similar results were obtained in vitro using human foreskin fibroblasts.

During the time course of the implantation study, the subcutaneously implanted hydrogels showed measurable fragmentation; ca. 25-30% of the overall hydrogel size. This result may indicate that the dextran-based hydrogels have the propensity to act as scaffolds for new cell growth and tissue formation in vivo. Hydrogels DS 28%, 31% and 47% were degraded between 5 and 10 days, 20 and 30 days, and >40 days, respectively. It is likely that the hydrogel degradation is mainly caused by the hydrolysis of the ester linkage of the crosslink molecules, as observed in the in vitro studies. After hydrogel degradation, either epidermis or dermis presented a cellular organization apparently similar to the one observed before implantation.

In summary, this work shows that selected commercially available crude protease and lipase preparations are able to produce hydrogels by a simple one-step reaction. This enzymatic approach enables the formation of macroporous, structurally organized hydrogels with superior mechanical properties to dextran-based hydrogels obtained chemically with the same starting materials. Furthermore, these hydrogels were in vivo biocompatible, degradable and presented interesting properties for their use as protein release systems, particularly, cationic proteins. These hydrogels may find interesting applications in the biomedical field as cell-carriers (scaffolds) or implantable protein/peptide delivery systems, specifically for the release of growth factors.

### REFERENCES FOR EXAMPLE 2

01—Saito, N.; Okada, T.; Horiuchi, H.; Murakami, N.; Takahashi, J.; Nawata, M.; Ota, H.; Nozaki, K.; Takaoka, K. A biodegradable polymer as a cytokine delivery system for inducing bone formation. Nature Biotechnol. 19, 332-335 (2001).

02— Zhu, G.; Mallery, S. R.; Schwendeman S. P. Stabilization of proteins encapsulated in injectable poly(lactide-co-glycolide). Nature Biotechnol. 18, 52-57 (2000).

03—Richardson, T. P.; Peters, M. C.; Ennett, A. B.; Mooney, D. J. Polymeric system for dual growth factor delivery. Nature Biotechnol. 19, 1029-1034 (2001).

04—Shea, L. D.; Smiley, E.; Bonadio, J.; Mooney, D. J. DNA delivery from polymer matrices for tissue engineering. Nature Biotechnol. 17, 551-554 (1999).

05—Griffith, L. G.; Naughton, G. Tissue engineering-current challenges and expanding opportunities. Science 295, 1009-1014 (2002).

06—Wong, W. H.; Mooney, D. J. Synthesis and properties of biodegradable polymers used as synthetic matrices for tissue engineering. In: Synthetic biodegradable polymer scaffolds; Atala, A., Mooney, D. J., Eds; Birkhauser, Boston, 1997, pp. 49-82.

07—Agrawal, C. M.; Ray, R. B. Biodegradable polymeric scaffolds for musculoskeletal tissue engineering. J. Biomed. Mater. Res. 55, 141-150 (2001).

08—Hooper, K. A.; Macon, N. D.; Kohn, J. Comparative histological evaluation of new tyrosine-derived polymers and poly(L-lactic acid) as a function of polymer degradation. J. Biomed. Mater. Res. 41, 443-454 (1998).

09—Holmes, T. C. Novel peptide-based biomaterial scaffolds for tissue engineering. TIBTECH 20, 16-21 (2002).

10—Mehvar, R. Dextrans for targeted and sustained delivery of therapeutic and imaging agents. J. Control. Release 69, 1-25 (2000).

11—Brøndsted, H.; Hovgaard, L.; Simonsen, L. Dextran hydrogels for colon-specific drug delivery II. Synthesis and characterization. Eur. J. Pharm. Biopharm. 42, 85-89 (1996).

12—Van Dijk-Wolthuis, W.N.E.; Franssen, O.; Talsma, H.; van Steenbergen, M. J.; Kettenes-van den Bosch, J. J.; Hennink, W. E. Synthesis, characterization, and polymerization of glycidyl methacrylate derivatized dextran. Macromolecules 28, 6317-6322 (1995).

13—Kim, S.-H.; Chu, C.-C. Synthesis and characterization of dextran-methacrylate hydrogels and structural study by SEM. J. Biomed. Mater. Res. 49, 517-527 (2000).

14—Zhang, Y.; Won, C.-Y.; Chu, C.-C. Synthesis and characterization of biodegradable network hydrogels having both hydrophobic and hydrophilic components with controlled swelling behavior. J. Polym. Sci. A. Polym. Chem. 37, 4554-4569 (1999).

15—Kim, S.-H.; Chu, C.-C. Pore structure analysis of swollen dextran-methacrylate hydrogels by SEM and mercury intrusion porosimetry. J. Biomed. Mater. Res. (Appl. Biomater.) 53, 258-266 (2000).

16—Meyvis, T.; De Smedt, S.; Stubbe, B.; Hennink, W.; Demeester, J. On the release of proteins from degrading dextran methacrylate hydrogels and the correlation with the rheologic properties of the hydrogels. Pharm. Res. 18, 1593-1599 (2001).

17—Tang, A.; Wang, C.; Stewart, R. J.; Kopecek, J. The coiled coils in the design of protein-based constructs: hybrid hydrogels and epitope displays. J. Control. Release 72, 57-70 (2001).

18—Zaks, A.; Klibanov, A. M. Enzymatic catalysis in nonaqueous solvents. J. Biol. Chem. 263, 3194-3201 (1988).

19—Almarsson, O.; Klibanov, A. M. Remarkable activation of enzymes in nonaqueous media by denaturing organic cosolvents. Biotechnol. Bioeng. 49, 87-92 (1996).

20—Ferreira, L.; Gil, M. H.; Dordick, J. S. Enzymatic Synthesis of dextran-containing hydrogels. Biomaterials 23, 3957-3967 (2002).

21—Martin, B. D.; Linhardt, R. J.; Dordick, J. S. Highly swelling hydrogels from ordered galactose-based polyacrylates. Biomaterials 19, 69-76 (1998).

22—Mak, A. F. T.; Zhang M. Skin and muscle. In: Handbook of biomaterial properties; Black, J. and Hasting, G., Eds.; Chapman & Hall: London, 1998; pp.66-69.

23—De Smedt, S. C.; Lauwers, A.; Demeester, J.; Van Steenbergen, M. J.; Hennink, W. E.; Roefs, S. P. F. M. Characterization of the network structure of dextran glycidyl methacrylate hydrogels by studying the rheological and swelling behavior. Macromolecules 28, 5082-5088 (1995).

24—Ferreira, L.; Gil, M. H.; Carvalho, R.; Geraldes, C. F. G. C.; Kim, D.-Y.; Dordick, J. S. Biocatalytic polytransesterification of inulin with divinyladipate. Chem. Materials 14, 4009-4011 (2002).

25—Dziubla, T. D.; Torjman, M. C.; Joseph, J. I.; Murphy-Tatum, M.; Lowman, A. M. Evaluation of porous networks of poly(2-hydroxyethylmethacrylate) as interfacial drug delivery devices. Biomaterials 22, 2893-2899 (2001).

26—Ferreira, L. Enzymatic modification of carbohydrates with vinyl monomers. Application to the preparation of hydrogels. PhD thesis dissertation. University of Coimbra, Portugal, 2003.

27—Wissink, M. J. B.; Beemink, R.; Pieper, J. S.; Poot, A. A.; Engbers, G. H. M.; Beugeling, T.; van Aken, W, G.; Feijen, J. Binding and release of basic fibroblast growth factor from heparinized collagen matrices. Biomaterials 22, 2291-2299 (2001).

28—Tanihara, M.; Suzuki, Y.; Yamamoto, E.; Noguchi, A.; Mizushima, Y. Sustained release of basic fibroblast growth factor and angiogenesis in a novel covalently crosslinked gel of heparin and alginate. J. Biomed. Mater. Res. 56, 216-221 (2001).

29—Kuijpers, A. J.; Engbers, G. H. M.; van Wachem, P. B.; Krijgsveld, J.; Zaat, S. A. J., Dankert, J.; Feijen, J. Controlled delivery of antibacterial proteins from biodegradable matrices. J. Control. Release 53, 235-247 (1998).

30—Patil, N. S. Synthesis, characterization and applications of hydrogels based on sugar acrylates. PhD thesis dissertation. University of Iowa, USA, 1997.

31—Lu, S.; Anseth, K. S. Release behavior of high molecular weight solutes from poly(ethylene glycol)-based degradable networks. Macromolecules 33, 2509-2515 (2000).

32—Kuijpers, A. J.; Wachem, P. B.; Luyn, M. J. A.; Plantinga, J. A.; Engbers, G. H. M.; Krijgsveld, J.; Zaat, S. A. J.; Dankert, J.; Feijen, J. In vivo compatibility and degradation of crosslinked gelatin gels incorporated in knitted Dacron. J. Biomed. Mater. Res. 51, 136-145 (2000).

33—Cadee, J. A.; Luyn, M. J. A.; Brouwer, L. A.; Plantinga, J. A.; van Wachem, P. B.; Groot, C. J.; Otter, W.; Hennink, W. E. In vivo biocompatibility of dextran-based hydrogels. J. Biomed. Mater. Res. 50, 397-404 (2000).

34—Loebsack, A.; Greene, K.; Wyatt, S.; Culberson, C.; Austin, C.; Beiter, R.; Roland, W.; Eiselt, P.; Rowley, J.; Burg, K.; Mooney, D.; Holder, W.; Halberstadt, C. In vivo characterization of a porous hydrogel material for use as a tissue bulking agent. J. Biomed. Mater. Res. 57, 575-581 (2001).

35—Holder, W. D.; Gruber, H. E.; Moore, A. L.; Culberson, C. R.; Anderson, W.; Burg, K. J. L.; Mooney, D. J. Cellular ingrowth and thickness changes in poly-L-lactide and polyglycolide matrices implanted subcutaneously in the rat. J. Biomed. Mater. Res. 41, 412-421 (1998).

36—Österberg, E.; Bergstrom, K.; Holmberg, K.; Schuman, T. P.; Riggs, J. A.; Burns, N. L.; Van Alstine, J. M.; Harris, J. M. Protein-rejecting ability of surface-bound dextran in end-on and side-on configurations: comparison to PEG. J. Biomed. Mat. Res. 29, 741-747 (1995).

37—Frazier, R. A.; Matthijs, G.; Davies, M. C.; Roberts, C. J.; Schacht, E.; Tendler, S. J. B. Characterization of protein-resistant dextran monolayers. Biomaterials 21, 957-966 (2000).

38—Massia, S. P.; Stark, J. Immobilized RGD peptides on surface-grafted dextran promote biospecific cell attachment. J. Biomed. Mater. Res. 56, 390-399 (2001).

39—Ferreira, L.; Ramos, M. A.; Gil, M. H.; Dordick, J. S. Exquisite regioselectivity and transesterification activity enhancement of an immobilized Bacillus subtilis protease. Biotechnol. Prog. 18, 986-993 (2002).

40—Washburn, E. W. Note on a method of determining the distribution of pore sizes in a porous material. Proc. Natl. Acad. Sci. 7, 115-116 (1921).

41—De Jong, S. J.; van Eerdenbrugh, B.; van Nostrum, C. F.; Kettenes-van den Bosch, J. J.; Hennink, W. E. Physically crosslinked dextran hydrogels by stereocomplex formation of lactic acid oligomers: degradation and protein release behavior. J. Control. Release 71, 261-275 (2001).

42—Bancroft, J. D.; Stevens, A. Theory and practice of histological techniques. Edinburg: Churchill Livingstone, 1996; pp. 99-211.

### Example 3

Biocompatability of Chemoenzymatically Derived Dextran-Acrylate Hydrogels

Overview

Example 1 herein describes a novel chemoenzymatic strategy for the preparation of dextran acrylates.¹ The biocatalytic approach was highly regioselective in the sites of acrylic ester modification of the dextran backbone and resulted in controlled degree of acrylate substitution. Hydrogels were obtained upon free radical polymerization of aqueous solutions of dextran-acrylate. These hydrogels may have suitable applications as implantable protein delivery systems,^{2,3} due to the highly stabilizing effect of polysaccharides on proteins and other biological macromolecules,^{4,5} as protein- and cell-resistant coatings⁶⁻⁸, and as bioactive scaffolds for tissue engineering.^{9,10} In this last case, the cell adhesive properties of the scaffold can be promoted by the immobilization of cell adhesion peptides targeted to specific cell types.^{11,12} Indeed, dextran has multiple binding sites along its chain which can incorporate high concentrations of bioactive molecules.⁸

Irrespective of their specific use, the biocompatibility of dextran-based hydrogels makes them attractive materials for implantables. Nevertheless, few studies have been performed in vitro¹³ and in vivo¹⁴ on biocompatibility of dextran-based hydrogels. The present study was undertaken to assess the biocompatibility of dextran-acrylate hydrogels that were synthesized chemoenzymatically. In vitro biocompatibility tests were performed using human foreskin fibroblasts, which are known to play a major role in cutaneous wound healing.^{15,16} Hydrogel biocompatibility was evaluated according to the extract assay, direct or indirect contact assays and cell-adhesion.^{13,17,18} In vivo biocompatibility and degradability were determined following subcutaneous and intramuscular implantation into Wistar rats for up 40 days. These results indicate that dextran hydrogels are biocompatible and the inflammatory and healing responses of rat tissues were influenced by the initial water content and the degree of substitution (DS) of the hydrogels.

Experimental Protocol

Materials

Dextran (from Leuconostoc mesenteroides, dexT70, Mₙ=39,940, M_{w}=70,000, according to the manufacturer's specification) was obtained from Fluka Chemie AG (Buchs, Switzerland). Dimethylsulfoxide (DMSO), N,N,N′,N′-tetramethylenediamine (TEMED) and ammonium persulfate (APS) were purchased from Aldrich (Milwauke, Wis., USA). Dextran acrylates (dexT70-VA) with different degree of substitution (DS) were synthesized as described previously.¹ The products were characterized by ¹H NMR to assess DS. Dulbecco's Modified Eagle Medium (DMEM) with glutamax-I (Gibco, UK) was supplemented with 10% fetal bovine serum (Gibco), 1% of fungizone (250 μg/mL of amphotericin B, Gibco) and 0.5% of gentamicin (10 mg/mL, Gibco). This medium is further referred as DMEM complete medium. MTT (3-[4,5-dimethylthiazol-2-yl]-2,5-diphenyl-tetrazolium bromide) was obtained from Sigma (St. Louis, USA). Transwell plates (6 wells) were purchased from Coming (NY, USA) and were formed by a suspended tissue culture treated polycarbonate membrane (24 mm diameter; 8.0 μm pore size) and a polystyrene plate. All other chemicals and solvents used in this work were of the highest purity commercially available.

Gel Preparation

Dext70-VA hydrogels were obtained by free radical polymerization of aqueous solutions of dexT70-VA as a function of DS and monomer concentration. Dext70-VA (80 or 200 mg) was dissolved in 0.9 mL of 0.2 M phosphate buffer, pH 8.0, and bubbled with nitrogen for 2 min. The polymerization reactions, performed in a cell culture plate (diameter≅1.8 cm), were initiated by adding 50 μL APS (80 mg/mL in 0.2 M phosphate buffer, pH 8.0) and 50 μL TEMED solution (13.6% (v/v) in water; pH adjusted to 8.0 with 12 N HCl), and allowed to proceed for 24 h at 25° C. The hydrogels synthesized contained an initial water content of 92% (w/w) and 80% (w/w), when 80 mg and 200 mg of dexT70-VA macromonomer were used, respectively.

Swelling Ratio Determination

Following removal of the hydrogels from the plate were immersed in ca. 50 mL of 0.01 M citrate-phosphate buffer, pH 7.0, changing the buffer daily, at 25° C. (in some cases, after removing the hydrogels they were dried and weighed to determine their initial dry weight). At regular intervals, the swollen gels were removed, blotted with filter paper to remove surface water, weighed, and returned to the same container until weight stabilization (Wₛ) was observed (normally up to 7 days). In some cases the hydrogels disks were steam-sterilized for 20 min at 120° C. followed by equilibration of the hydrogels for 24 h at 25° C. The hydrogels were then dried at room temperature, under vacuum, in the presence of phosphorous pentoxide (until constant weight was achieved), and weighed to determine the dry weight, W_{d}. The swelling ratio at equilibrium (SRE) was calculated according to eq. (1):

$$SRE = \frac{{W}_{s} - {W}_{d}}{{W}_{d}} \qquad ( 1 )$$

The gel-fraction of hydrogels was calculated from the ratio of the dry weight of hydrogel after swelling and the initial dry weight immediately after the polymerization reaction. The sol-fraction was calculated from the subtraction of 1−gel fraction.

In vitro Biocompatibility Tests

Cell culture. Primary human skin fibroblasts were grown in DMEM complete medium, at 37° C. in a fully humidified air containing 5% CO₂ (IR auto Flow). The cells were fed every 2 to 3 days. When cells reached confluence, the culture medium was discarded and the cells washed with 5 mL of 10 mM phosphate buffered saline (PBS) pH 7.4 (Gibco). The cells were then detached with 2 mL of 0.05% (w/v) trypsin (1:250, from porcine pancreas (Sigma)) solution (PBS supplemented with 0.1% and 0.25% of α-D(+) glucose and EDTA, respectively) for 5 min at 37° C., and 3 mL of DMEM complete medium was added to inactivate the trypsin after cell detachment. The cells were centrifuged (10 min, 2500 rpm) and resuspended in culture medium before use. Cultures between the 3^{rd} and 7^{th} passages were used in the entire work.

Solutions of dextran, dexT70-VA, TEMED and APS. All solutions were prepared on the day of application. Solutions of 10, 20, 50 and 100 mg of dextran or dexT70-VA (DS 7.2% or DS 12.1%) per mL of DMEM complete and solutions of 2.5, 10, 30 and 50 μL of APS or TEMED per mL of DMEM complete medium were sterilized through a 0.22 μm filter (Schleicher & Schuell, Dassel, Germany), with the exception of 100 mg/mL dexT70-VA solutions where a 0.45 μm filter (Schleicher & Schuell) was used due to the higher viscosity.

Extraction assay. The extraction assay was performed in two sets of hydrogels. In one set, the hydrogels obtained after polymerization of dextran-acrylate were extracted for 2 days in 10 mM citrate-phosphate buffer pH 7.0 (3×40 mL), at 25° C., and afterwards autoclaved. In the second set the hydrogels obtained after the polymerization reaction were immersed in 10 mL of 10 mM citrate-phosphate pH 7.0 and immediately autoclaved. Extracts were obtained by immersing autoclaved hydrogels in DMEM (without phenol red) culture medium supplemented with 1% of fungizone and 0.5% of gentamicin, at 37° C., for 5 days with agitation (120 rpm). The ratio between the surface area of the material and the volume of extraction medium was 3 cm²/mL. Following incubation the medium containing the extract was collected, filtered (0.22 μm; to eliminate the possible presence of solid particles of the material) and supplemented with 10% of serum. Human skin fibroblasts were plated in 96-well plates (TPP, Switzerland) and grown to subconfluency. The culture medium was removed and replaced with the extract media for 24 h at 37° C. Phenol (64 g/L, BDH) which is considered cytotoxic¹⁷, was used as a positive control. Culture medium without extracts, incubated as described above, was used as a negative control. Following incubation the extracts were discarded and the mitochondrial metabolic activity of the cells was measured with the MTT assay. To test the cytotoxicity of dextran, dexT70-VA, TEMED, and APS solutions (see above), 100 PL of each test solution were added to each well plated with subconfluent cells.

Cell proliferation inhibition index (CPII) assay: direct contact assay. The CPII assay was performed according to De Groot et al. 13, which allows cytotoxicity evaluation on growing cells. To evaluate the CPII for solutions of dextran, dexT70-VA, TEMED, and APS, 100 μL of a fibroblast suspension containing 1.55×10⁴ cells/mL was plated into each well of a 96-well plate. Since each well has a surface area of 0.31 cm², the final seeding density was ca. 5×10³ cells/cm². After 4 h (the cells were adherent to the well bottom), the culture medium was discarded and 200 μL of each test solution was added to each well. For the control culture, the medium was refreshed. Seventy-two hours after the addition of test solutions, the extracts were discarded and the cell layer was washed with PBS to remove remaining materials and loose cells. The metabolic activity of the cells was then measured with the MTT test (see below). The CPII was calculated using eq. 6:

$$CPII = 100 - ( \frac{{OD}_{540} of test culture}{{OD}_{540} of control culture} \times 100 ) \qquad ( 6 )$$

To evaluate the CPII for dexT70-VA hydrogels (extracted for 3 days in water at 25° C. and then sterilized by autoclave), 3 mL of a fibroblast suspension containing 1.5×10⁴ cells/mL was plated into each well of a 6-well plate, to yield a final density of 5×10³ cells/cm². After 4 h, the culture medium was refreshed (3 mL) and the hydrogels were added to the wells, in direct contact with cells (FIG. 1A). For the control culture, the culture medium was refreshed. After 72 h of incubation, the metabolic activity of the cells was assessed as previously described and the CPII calculated according to eq.6.

CPII assay: indirect contact assay. The CPII of hydrogel samples was also evaluated using transwell plates, containing a final density of 5×10³ cells/cm² (FIG. 1B). In this case, after polymerization (without any extraction), the hydrogels were sterilized by autoclave, washed twice with DMEM complete medium, and seeded into the wells in indirect contact with the cells (total of 4 mL culture medium). After 72 h of incubation, the metabolic activity of the cells was determined as described for the direct contact assay, and the CPII calculated according to eq.6.

Cell adhesion assay. Filtered (0.45 μm) dexT70-VA solutions (450 μL) were placed into each well of a 24-well plate and polymerized by addition of filtered APS (25 μL) and TEMED (25 μL) solutions, in sterile conditions. After 24 h of polymerization, the hydrogels covering the bottom of the wells were washed with DMEM complete medium (3×450 μL) and 1.0×10⁴ cells/cm² were seeded onto the surface. After 24 h of incubation, the hydrogels were washed with PBS (2×450 mL) and the cells trypsinized and counted using a cell counting chamber (Nfubauer, Germany).

Mitochondrial metabolic activity assay. For 96-well plates, the cell layers were rinsed with PBS (110 μL) and 110 μL of MTT (0.45 mg/mL in DMEM complete medium w/o phenol red) was added to each well. For 6-well plates, after PBS washing (1 mL), 2.5 mL of MTT solution was added. After 3 h incubation at 37° C., the MTT solution was removed and the insoluble formazan crystals formed in the bottom of the wells were dissolved in 100 μL or 1 mL of DMSO, for 96 and 6-well plates respectively. The absorbance was measured at 540 nm using a plate reader (STL Spectra III, Austria).

In vivo Biocompatibility Studies

Animals. Male Wistar rats (8-10 weeks old) were obtained from the Faculty of Medicine of Coimbra University and used for all studies. Rats were given standard feed and water ad libitum and were on a 12 h light/dark cycle. European community guidelines (n° 86/609/CE; corresponding to decree n° 1005/92 of Portuguese legislation) for the care and use of laboratory animals were observed.

Implantation studies. Aseptic techniques were used for all surgical procedures. Animals were anesthetized, with Ketalar® (50 mgKg⁻¹, Parke-Davis) and an area of the back and front was shaved and washed with Betadine®. In the front, two incisions along the spine (ca. 1 cm in length) were made and two subcutaneous pockets were created. The gels (3 mm×2 mm, 3 mm thickness) were placed into the pocket away from the incision (ca. 0.5 cm) and the skin was closed with Mersilk® non-absorbable suture (3 Øs, Ethicon). In the back, two incisions along the spine (ca. 1 cm in length) were made and a further incision was performed in each case in the skeletal muscle (Gluteus superficialis) to implant the gel. The muscle incision was closed with sterile polypropylene suture (6 Øs, Ethicon) and the skin incision closed with Mersilk® suture (see above). The two areas were washed with Betadine® dermic solution and the rats kept warm with a heating pad for 1 h after the surgical procedure and finally transferred to a cage. Three rats were used for each time point. As controls, subcutaneous and intramuscular pockets were made without implants.

Histological examination. The rats were sacrificed and the implants with surrounding tissue where carefully dissected and fixed in 4% (v/v) neutral buffered formalin, for at least 3 days. The blocks were sliced perpendicular or cross-sectional to the implanted hydrogels. The implant and adjacent tissue were oriented and placed in processing cassettes, taken through a graded ethanol series (Shandon Citadel 1000) and embebbed in paraffin. The samples were then sectioned using a microtome (Shandon Retraction AS 325), and finally deparaffinized and stained either with hematoxylin/eosin (HE), periodic acid-schiff (PAS) or Masson's trichrome (MT)¹⁹. Multiple photographs were taken of each hydrogel and the surrounding tissues using a Nikon microscope (Eclipse E 600) with a Nikon camera (FOX-35). The tissue response was rated by two persons. Capsule thickness was measured on nine fields per section, obtained from different blocks.

Statistical Analysis

One-way ANOVA with Bonferroni post test was carried out for statistical tests by using GraphPad Prism 3.0 (San Diego, Calif., USA) software package. A p value of <0.01 was considered to be statistically significant.

Results

Several DexT70-VA hydrogels were prepared chemoenzymatically as described previously.¹ Equilibrium swelling ratio (SRE) and gel fraction were dependent on both the initial water content of the gel and the degree of substitution (DS), as well as further treatment by autoclaving. As shown in Table 6, the gel fractions of non-sterilized dexT70-VA hydrogels were between 59 and 83%, which indicates that incubation of these networks in water at 37° C. leads to considerable release of polymeric components.

TABLE 6
Characteristics of dexT70-VA hydrogels.

| | Gel Fraction^{a} | | | |
|---|---|---|---|---|
| DexT70-VA | (%) | | SRE^{d} | |
| hydrogel | BS^{b} | AS^{c} | BS^{b} | AS^{c} |
| 92%, DS 7.2% | 59.0 | 55.7 | 31.5 | 36.6 |
| 92%, DS 12.1% | 74.5 | 74.3 | 14.4 | 14.4 |
| 92%, DS 22.4% | — | — | 11.0 | 11.5 |
| 92%, DS 31.5% | — | 77.8 | 9.78 | 10.6 |
| 80%, DS 7.2% | 80.2 | 78.5 | 7.32 | 10.2 |
| 80%, DS 12.1% | 83.0 | 84.7 | 4.39 | 5.86 |
| 80%, DS 22.4% | — | — | 3.99 | 5.32 |
| 80%, DS 31.5% | — | 81.1 | 3.72 | 4.80 |

^{a}The hydrogels were extracted in water for 5 days (in some cases further sterilized by autoclave), dried and weighed. The final dry weights were compared with the sample initial dry weight (immediately after the polymerization reaction) to calculate the gel fraction. Average of two independent measurements.
^{b}Before sterilization by autoclave.
^{c}After sterilization by autoclave.
^{d}Swelling ratio at equilibrium. Average of two independent measurements.

Subsequent autoclaving did not alter significantly the gel fraction and thus showing that this step did not hydrolyze the hydrogel network. SRE values ranged from 3.7 to 31 for non-sterilized hydrogels (Table 6). In addition, the treatment by autoclaving slightly increased the SRE, particularly in hydrogels with low initial water content (80%).

In vitro Biocompatibility

Cytotoxicity Assays

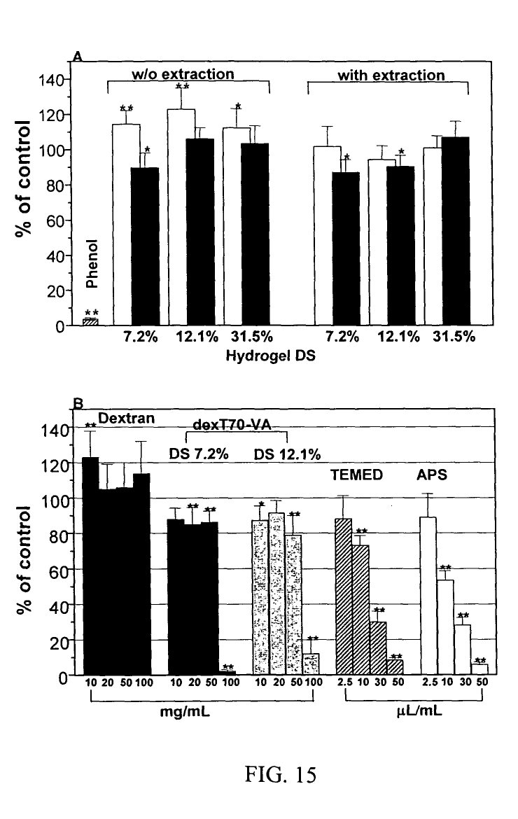

To identify whether components of the hydrogel synthesis procedure were cytotoxic, subconfluent human skin fibroblasts were exposed to extracts of hydrogels prepared with different DS values and initial water contents. Cellular viability was quantified using the standard MTT assay.²⁰ Two sets of hydrogels were used for the extraction assay. In one set, the hydrogels were extracted in 10 mM citrate-phosphate buffer, pH 7.0, for 2 days and then autoclaved. In the second set, the hydrogels obtained after polymerization were immediately autoclaved. Afterwards, both sets of hydrogels were extracted in cell culture medium for 5 days at 37° C. and the extracts incubated with fibroblast cultures for 24 h. The results of the MTT assays (FIG. 15A) show that the extracts of all hydrogels induce less than 20% change in the mitochondrial metabolic activity of fibroblasts (MMAF), as compared to the control. Non-extracted hydrogels with an initial water content of 92% exerted a statistically significant increase in MMAF (10-20%); however, no effect was observed in extracted hydrogels. In some cases, extracted or non-extracted hydrogels with an initial water content of 80% induced a ca. 10% reduction in MMAF.

Dextran hydrogels can release leachable products during the extraction assay (Table 6) and, therefore, the cytotoxicity of individual hydrogel components was evaluated. Solutions of dextran, dexT70-VA monomers with different DS values (7.2 and 12.1%), APS, and TEMED were incubated with fibroblast cultures for 24 h under the same conditions as described for dexT70-VA hydrogel extracts, and the cellular viability assessed by the MTT assay (FIG. 15B). Dextran solution at a concentration of 10 mg/mL induced a statistically significant (P<0.001) increase in the MMAF (ca. 20%), although this effect was not significant in the concentration range of 20-100 mg/mL. In contrast, dexT70-VA monomers with different DS values slightly reduced (10-20%) the MMAF at 50 mg/mL; however, did so extensively (>80%) at concentrations of 100 mg/mL. Finally, TEMED and APS exerted a significant (P<0.001) decrease in MMAF for concentrations above 2.5 μL/mL.

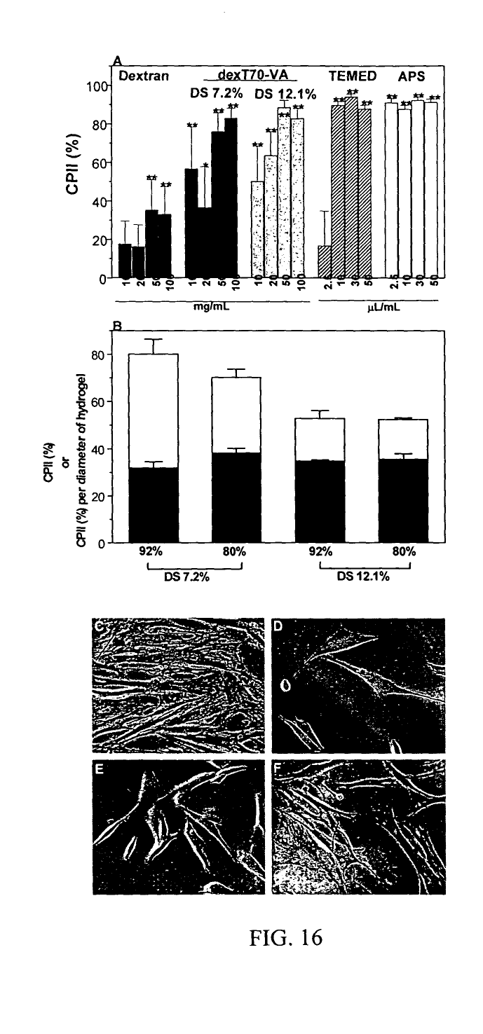

Cytotoxicity studies were extended to evaluate the cellular proliferation inhibition index (CPII). To that end, the toxicity of either hydrogels or single components of the hydrogels was evaluated in actively growing cell culture for 72 h. As shown in FIG. 16A, the CPII values of dextran solutions in the concentration range of 10-20 mg/mL were ca. 18%, not statistically different from the control; however, at higher concentrations the CPII significantly (P<0.001) increased to ca. 35%. CPII values of dexT70-VA monomers with DS 7.2% and 12.1% were between 36 and 64% for a concentration range of 10-20 mg/mL, and this was increased to ca. 80% at higher concentrations. Finally, APS caused a significant reduction in the MMAF for all concentrations (CPII values of ca. 90%), albeit with TEMED the same phenomenon was observed above 2.5 μL/mL.

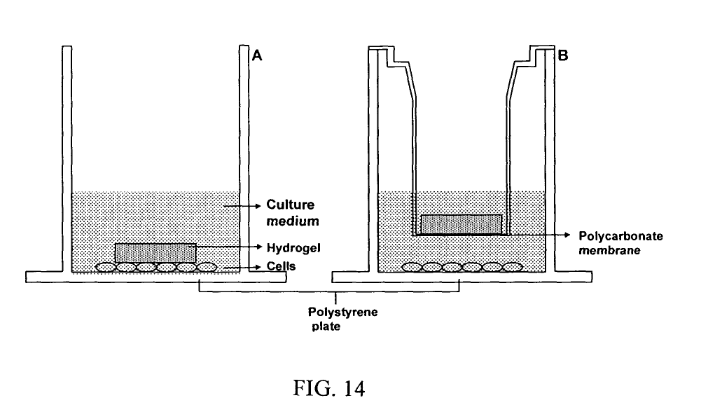

The cytotoxicity of dexT70-VA hydrogels was also assessed by the CPII test using a direct contact method (FIG. 16B). Specifically, the hydrogels were seeded into the cellular layer of a 6-well plate (FIG. 14A). DexT70-VA hydrogels with low DS (7.2%) yielded higher values of CPII (70-80%) than high DS hydrogels (ca. 52%). Since the hydrogels have different diameters after swelling, the CPII was normalized per diameter of the hydrogel. In this case, the CPII values were identical which indicates that hydrogel size played an important role.

Light microscopy examination was also undertaken to characterize the morphology of the cells under and in the proximity of the hydrogels (FIGS. 16C, 16D, 16E, and 16F). Cells in the proximity of the hydrogels appeared to have normal morphology, as compared to the control (FIG. 16C), and were well-spread on the polystyrene matrix (data not shown). However, for dextran hydrogels with low DS (7.2%), independent of the water content, the cells underneath the hydrogels were less elongated and less well-spread (FIGS. 16D and 16E). In contrast, the cells under the 80% dexT70-VA DS 12.1% hydrogel (FIG. 16F) did not present a significantly different morphology, as compared to the control cells albeit they were lower in number.

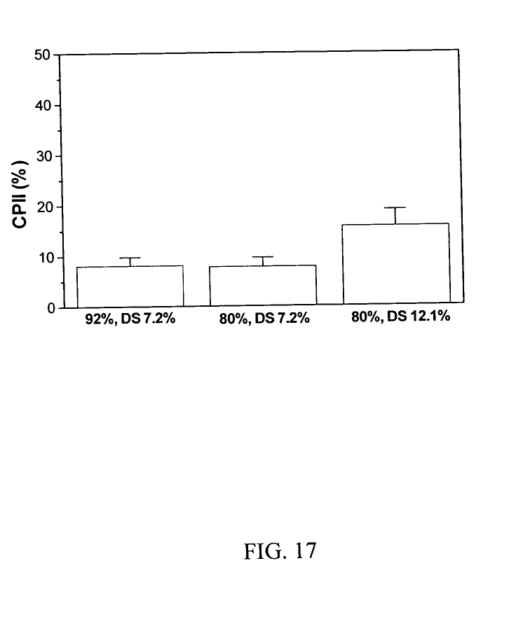

To assess whether the high values of CPII obtained by the direct contact assay were truly due to hydrogel toxicity or to other side effects, an indirect contact assay (transwell experiment, FIG. 14B) was performed. As shown in FIG. 17, the CPII decreased to values below 16% and were not statistically different from the control.

Cell-adhesion Assay

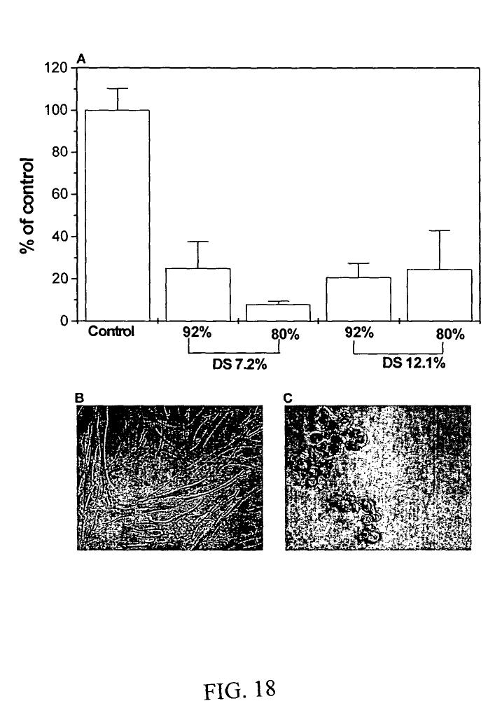

Cell adhesion onto hydrogels was evaluated and expressed as a percentage of control adhesion on tissue culture polystyrene (TCPS) (FIG. 18A). Fibroblast adhesion was reduced in all hydrogels tested with different DS values and initial water contents (below 28%). Because dextran hydrogels were transparent, cell morphology was evaluated by phase-contrast inverted light microscopy (FIGS. 18B and 18C). After 24 h of cell incubation in the presence of the hydrogels, the relatively few cells attached were rounded and formed clusters without filopodia to anchor the cells to the hydrogels, as observed by scanning electron microscopy (data not shown).

In vivo Biocompatibility

DexT70-VA hydrogel samples were subcutaneous and intramuscularly implanted in rats and the intensity of the inflammatory response to the foreign implants was monitored by histology at varying implantation times. Table 7 provides an overview of the nature and extent of the observed tissue reaction after implantation of hydrogels with different initial water contents and DS values.

Subcutaneous Implantation

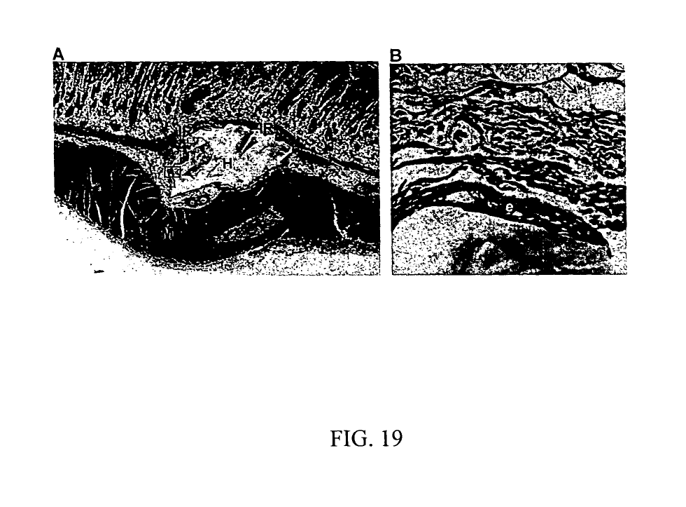

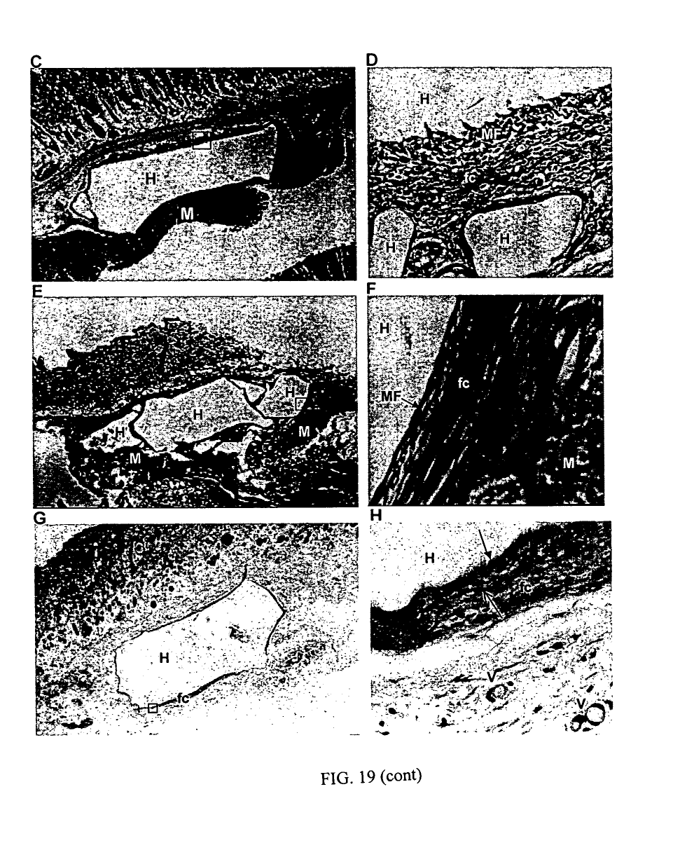

At day 2, 80% dexT70-VA DS 7.2% was mainly surrounded by fibroblasts. Some lymphocytes, but no granulocytes, were observed (FIGS. 19A and 19B). Fibrin and exudate were also identified in the proximity of the hydrogel. In contrast, the other dextran hydrogels did show the presence of granulocytes to different extents, which were attached to the interface of the hydrogel/tissue. Typically, dextran hydrogels with high initial water content attracted higher numbers of granulocytes, and also yielded greater vascularization.

At day 5 after implantation, the cell layer surrounding the 80% dexT70-VA hydrogel DS 7.2% consisted primarily of fibroblasts but also of smaller fractions of granulocytes and lymphocytes. The other dextran hydrogels did not present granulocytes. In 80% dexT70-VA DS 22.4% hydrogel some foreign-body giant cells were already observed.

At day 10, the start of fibrous capsule was observed in the 80% dexT70-VA DS 7.2% hydrogel, and macrophages and fibroblasts were found between this capsule and the hydrogel (FIGS. 19C and 19D). No granulocytes, exudate and fibrin were found at this stage. Lymphocytes were observed only occasionally and the number of blood vessels remained approximately constant as compared to the previous postimplantation time. Similar tissue reactions were observed for the other dextran hydrogels. For 92% dexT70-VA DS 7.2% hydrogel the fragmentation was higher than that observed for the other hydrogels (in general restricted to the outer limits), likely due to mechanical stress on this relatively soft hydrogel. Finally, giant cells were observed for hydrogels with lower initial water contents (80%), while they were absent in hydrogels with higher initial water contents (92% dexT70-VA DS 7.2%).

At day 30, 80% dexT70-VA DS 7.2% hydrogel was surrounded by a thin layer of macrophages and fibroblasts and a fibrous capsule had formed around this layer (thickness of ca. 54.50±23.28 μm, n=2) (FIGS. 19E and 19F). The same profile was observed in the other hydrogels, and the interface of the hydrogel/tissue was less vascularized than at day 10. Finally, in the 92% dexT70-VA DS 7.2%, the fragments were surrounded by a thin discontinuous capsule, while on 80% dexT70-VA DS 22.4% hydrogel giant cells were found.

At day 40, 80% dexT70-VA DS 7.2% hydrogel (FIGS. 19G and 19H) was covered by one or two layers of macrophages and fibroblasts including some giant cells. This cell layer was surrounded by a thin fibrous capsule (35.14±21.58 μm, n=4). Similar tissue reactions were observed for the other dextran hydrogels, except for 92% dexT70-VA DS 7.2% hydrogel where the network was highly fragmented and no continuous fibrous capsule was observed. The fragments were surrounded mainly by fibroblasts and no giant cells were observed. Furthermore, the cells appeared to begin to invade the hydrogel.

Intramuscular Implantation

After intramuscular implantation of hydrogels (Table 7), the observed tissue reactions were more severe than those described for subcutaneous implantation.

TABLE 7
Tissue reactions to dexT70-VA hydrogels with different degrees of substitution (DS) and initial water
content implanted subcutaneous (SC) and intramuscularly (IM).

| | | | | Mononuclear cells | | | | | | | | | | | | | | | |
|---|---|---|---|---|---|---|---|---|---|---|---|---|---|---|---|---|---|---|---|
| | Time | PMN | | MØ³ | | Lymph.⁴ | | F⁵ | | Fibrin | | Exudate | | Necrosis | | Vascularization | | Haemorrhage | |
| Hydrogel | (Days) | SC | IM | SC | IM | SC | IM | SC | IM | SC | IM | SC | IM | SC | IM | SC | IM | SC | IM |
| 92% DS | 2 | ++ | + | − | −/± | ± | ± | + | + | + | + | + | + | − | + | ++ | ± | ± | + |
| 7.2% | 5 | − | − | − | − | ± | ± | ++ | ++ | − | − | − | − | − | + | ± | ± | − | ± |
| | 10 | − | − | + | +² | − | − | ++ | ++ | − | − | − | − | − | ± | ± | ±/+ | − | ± |
| | 30 | − | * | ± | * | − | * | + | * | − | * | − | * | − | * | −¹ | * | − | * |
| | 40 | − | − | − | ± | − | − | + | ± | − | − | − | − | − | − | ± | ± | − | − |
| 80% DS | 2 | − | ++ | − | − | ± | ± | ++ | ± | ± | + | ± | + | − | + | + | ± | − | + |
| 7.2% | 5 | ± | − | − | − | ± | ± | ++ | ++ | ± | ± | ± | − | − | + | ± | ± | ± | ± |
| | 10 | − | − | +² | +² | ± | ± | ++ | ++ | − | − | − | ± | − | ± | ± | + | − | + |
| | 30 | − | − | + | ±² | − | − | + | + | − | − | − | − | − | − | −¹ | ± | − | − |
| | 40 | − | − | ±² | ±² | − | − | + | + | − | − | − | − | − | − | −¹ | −/± | − | − |
| 92% DS | 2 | ++ | + | − | −/± | ± | ± | + | + | + | + | + | + | − | + | ++ | ± | ± | + |
| 22.4% | 5 | − | − | + | ±² | ± | ± | ++ | ++ | ± | − | − | − | − | ± | + | −¹ | + | − |
| | 40 | − | − | ±² | ±² | − | − | + | ± | − | − | − | − | − | − | ± | −/± | − | − |
| 80% DS | 2 | ± | ++ | − | − | ± | ± | + | − | ± | ++ | + | + | − | + | + | ± | − | + |
| 22.4% | 5 | − | − | +² | −/± | ± | ± | ++ | ++ | ± | ± | − | ± | − | ± | + | ± | + | ± |
| | 10 | − | − | +² | +² | − | ± | ++ | ++ | − | − | − | + | − | ± | ± | ±/+ | − | + |
| | 30 | − | − | +² | +² | − | − | + | + | − | − | − | − | − | − | −¹ | ± | − | ± |
| | 40 | − | − | ±² | ±² | − | − | + | + | − | − | − | − | − | − | ± | − | − | − |

± to +++ = sporadic to severe;
− = not present;
PMN = Polymorph nucleocytes cells, i.e., granulocytes.
¹Regular number as compared to a normal tissue without any implantation.
²It includes some multinucleated cells (giant cells).
³Macrophages.
⁴Lymphocytes.
⁵ Fibroblasts.
*Not available.

At an early stage (day 2), an intense inflammatory process was observed at the implantation site with 80% dexT70-VA DS 7.2%. Granulocytes and mononucleated cells infiltrated into muscle tissue, and muscle cell necrosis (as measured by the presence of “ghost cells”, i.e., weakly stained cells) were observed. Similar tissue reactions were found for the remaining hydrogels; however, hydrogels with lower water contents (80%) showed slightly higher initial tissue response than dextran hydrogels with higher water contents (92%), as indicated by the increased infiltration of granulocytes (Table 7) into the implantation area. To check whether this response was due to the implant or to the extent or degree of defect created by the implantation procedure, histologic evaluation on rats with unfilled surgical implant was performed. Tissue responses with the same intensity and extent were observed, indicating that the procedure of implantation had a more significant effect on tissue response than the biomaterial itself.

The aforementioned tissue reaction had decreased by day 10, and granulocytes originally at the implantation site were replaced by mononucleated cells with the start of fibrous capsule observed. At days 30 and 40, the cellular infiltration of muscle cells was delimited to small areas and the necrosis process ceased. A thin fibrous capsule (<40 μm) was observed at days 30 and 40 for all hydrogels tested with the exception of 92% dexT70-VA DS 7.2% hydrogel, where a continuous fibrous capsule was not observed even after 40 days. At these implantation times the hydrogels were surrounded by fibroblasts, macrophages, and some giant cells. Higher numbers of giant cells were observed in dextran hydrogels with lower initial water contents (80%).

In general, the fragmentation process was large in 92% dexT70-VA DS 7.2% hydrogel, reaching in some cases ca. 80% of the overall area. This process was minimal with the other hydrogels, normally occurring only at the outer limits of the gel networks. As expected, the hydrogel fragmentation was higher for intramuscular than subcutaneous implantation, presumably due to the mechanical stress promoted by the movement of the animal.

To assess the degradability of hydrogels the tissue surrounding hydrogels was stained with Periodic Acid Schiff (PAS).^{14,19} All tissue sections (subcutaneous or intramuscular implants) were PAS staining negative, which indicated that cells did not uptake (e.g. by phagocytosis) the hydrogels, during the time frame of this study.

Discussion

The information obtained from sol-gel fractions is important for biocompatibility assessment, as either unreacted monomers or polymerization initiators may be cytotoxic. Our results show that hydrogels prepared with lower initial water contents in the polymerization reaction had higher gel fractions, and consequently lower sol fractions (i.e., unreacted monomers that can be released from the hydrogel). This is not surprising since the reaction of highly concentrated solutions of dextran acrylate monomers is favored over low concentrated ones.¹

The likely leachable products from dexT70-VA hydrogels showed different in vitro biocompatibility profiles. Dextran had a minimal effect in cell viability. The only effect was observed at low concentrations (10 mg/mL) and consisted of a slight increase in MMAF. This effect can be due to the increase of either cell viability or the mitochondrial succinic dehydrogenase enzyme activity (responsible for MTT metabolism) or possibly both by an unknown mechanism. Regarding cell proliferation, dextran exhibited a relative inhibition of 18-35% depending on its concentration. Similar CPII values (25±7%) were reported previously¹³ for dextran with a molecular weight of 40 kDa (100 mg/mL) using human skin fibroblasts and using similar conditions as used in the current study.

Considering that the sol fraction of hydrogels was below 22% and 44% (Table 6) for 80% and 92% dexT70-VA hydrogels, respectively, the maximum amount of dexT70-VA released will be <43 mg/mL. In this concentration range, dexT70-VA monomer with different DS values slightly decreased (10-20%) cell viability; however, exerted a pronounced effect on cell proliferation, which was significantly different from that exhibited by dextran itself. This effect may be related to the presence of vinyl groups that may interfere with cell proliferation. Finally, as expected, either APS or TEMED reduced substantially cell viability and dramatically cell proliferation. This effect was expected, as free radicals can react with biological molecules (lipids, proteins, carbohydrates and nucleic acids) and thus interfere with cell viability and proliferation.²¹

Dextran hydrogels showed good in vitro biocompatibility,. Using the extraction assay, dextran hydrogels did not reduce cell viability by more than ca. 10% (suggesting that APS and TEMED were not released during extraction), and this effect was only observed with hydrogels prepared with an initial water content of 80%. Interestingly, non-extracted hydrogels with an initial water content of 92% induced a significant increase in the MMAF. This may be due to the release of small amounts of dextran (or dexT70-VA that is degraded in solution into dextran by hydrolysis of the acrylate ester linkages) from these hydrogels, which increases the MMAF as already demonstrated in hydrogel component cytotoxicity tests. Unexpectedly, the CPII assay (direct contact) results showed that these polymeric networks exerted pronounced cell proliferation inhibition (53-80%) when compared to the control, and this effect was dependent on the size of hydrogels. Hydrogels with larger diameters presented higher CPII; however, the differences among hydrogels were found to be negligible upon normalizing the CPII as a function of the hydrogel diameter. In addition, differences in cell morphology and number were observed for cells underneath the hydrogels but not in their proximity. This effect may be related to mechanical stress of the hydrogels on the cells or poor O₂/CO₂ exchange due to the physical presence of the network. Indeed, the CPII values obtained by the indirect contact assay were <16% and not significantly different from the control. Hence, dextran hydrogels only slightly reduced cell viability (<10%) and cell proliferation (<16%) and, therefore, may be considered as non-cytotoxic polymer networks.

The study of dextran hydrogel interaction with fibroblasts showed that the hydrogels were non-adhesive compared to TCPS. This resistance to cell adhesion was presumably due to poor protein adsorption^{6,7} onto the hydrophilic and nonionic dextran. Similar results were obtained by Massia & Stark⁸ in surfaces grafted with dextran, using endothelial and smooth muscle cells, and 3T3 fibroblasts. Moreover, these authors reported cell adhesion values below 26% for all types of cells, which agrees well with our results. The cell adhesion resistance of dextran hydrogels should not be interpreted as a sign of non-biocompatibility, as other polymers such as PEG have similar properties yet are widely recognized as biocompatible.¹¹

Finally, we demonstrated that dextran hydrogels are biocompatible in vivo, as determined through subcutaneous and intramuscular implantation studies in rats. The inflammatory and healing responses of rat tissues were influenced either by the implantation process (subcutaneous versus intramuscular) or the DS values and initial water content of the hydrogels. After subcutaneous implantation of dexT70-VA hydrogels, the hydrogels with higher water contents (92%) showed slightly higher inflammatory response, as expressed by a higher number of attached neutrophils, when compared to hydrogels with lower water contents (80%). It is well-known that the primary role of neutrophils is phagocytosis, and these cells are attracted to the implantation site by several factors, including chemoattraction due to the coating of the foreign body with opsonins.²² We speculate that the higher number of neutrophils in the proximity of hydrogels with higher water contents may be due to the more open structure of these hydrogels¹ as compared to lower water content hydrogels, and this would likely promote the entrapment of opsonins. Furthermore, the release of leachable products, due to a higher fragmentation process in these hydrogels (mainly in 92% dexT70-VA DS 7.2% hydrogel), may attract a high number of granulocytes.

After 10 days, a normal wound healing response to subcutaneously implanted hydrogels occurred, which varied according to the properties of the hydrogel. Hydrogels with higher DS values presented a higher number of foreign-body giant cells (FBGC) than those with lower DS values. FBGC are formed by fusion from macrophages²³ and that process is dependent on the form, composition, and topography of the implanted surface.^{15,24} In general, rough surfaces induce higher FBGC formation than smooth and flat surfaces.^{24,25} According to light microscopy observations, the surface of dextran hydrogels with higher DS values was rougher than those with lower DS values, and this may explain the higher number of FBGC observed in those hydrogels. Furthermore, the wound healing response was also characterized by the formation of a fibrous capsule involving the hydrogel, except for 92% dexT70-VA DS 7.2% hydrogel. In this case, a higher degree of hydrogel fragmentation affected the formation of a continuous fibrous capsule, and the surrounding cells began to invade the hydrogel, which is a typical response of biocompatible polymers that do not undergo biodegradation or bioresorption.^{15,16} The thickness of the fibrous capsule at day 40 (below 55 μm for all hydrogels) is comparable to²⁶ or slightly lower^{27,28} than other biocompatible materials described in the literature when implanted subcutaneously into rats during ca. 6 weeks. Indeed, this parameter is important in evaluating the performance of implantable drug delivery systems and scaffolds, as thick fibrous capsule may impede the diffusion of therapeutic substances and prevent effective implant integration. Along those lines, it has been demonstrated that fibrous capsule thickness of 10-35 μm does not circumvent the release of insulin from poly(hydroxyethyl methacrylate) sponges implanted in rats.²⁹ Therefore, it is likely that the capsule thickness values obtained herein would not interfere in therapeutic protein release from hydrogels. Further studies are now underway to demonstrate protein release from implanted dextran-based hydrogels.

The tissue response by intramuscular implantation of hydrogels was more severe than the subcutaneous implantation. The degeneration process during the first 5-10 days was mostly likely ascribed to surgical trauma³⁰ caused by the intramuscular implantation, and this was confirmed by similar tissue reaction in the unfilled surgical implant site control. Therefore, the higher degree of surgical trauma during the first 10 days precludes our ability to clearly interpret the biological response to the hydrogels. At days 30 and 40, the tissue response showed the formation of a thin fibrous capsule (<40 μm) for all hydrogels excluding 92% dexT70-VA DS 7.2% hydrogel, as well as minor and well-confined infiltration of fibroblasts and macrophages at the muscle tissue, likely due to the movement of the hydrogel relatively to the tissue. Thus, dextran hydrogels present acceptable muscle tissue compatibility. Finally, after ca. 6 weeks of either subcutaneous or intramuscular implantation, the hydrogels did not show signs of degradation. Hence, the acrylate ester linkages are stable at physiologic pH and the presence of esterolytic enzymes in tissues. This agrees with previous studies showing the non-degradability of dextran-methacrylate hydrogels¹⁴ obtained by chemical routes.

In summary, in vitro biocompatibility studies have demonstrated that chemoenzymatcally-generated dextran-based hydrogels do not significantly promote cell adhesion and minimally impact cell viability and proliferation. These hydrogels, therefore, may be considered non-cytotoxic. In vivo studies indicated that all hydrogels elicited a mild inflammatory response after subcutaneous implantation; however, intramuscular implantation resulted in trauma, which partially masked a potential inflammatory response from hydrogel implantation. Nevertheless, the foreign body reaction was normal for subcutaneous and intramuscular implantation and varied according to the DS value and initial water content of hydrogels. Thus dextran hydrogels can be considered as biocompatible networks, since the cellular response after implantation was normal, the fibrous capsule surrounding hydrogels had a thickness similar to those of other biocompatible materials, and neither damage nor necrosis of the surrounding tissues of the implant was observed.

### REFERENCES FOR EXAMPLE 3

01—Ferreira L, Gil MH, Dordick JS. Enzymatic Synthesis of dextran-containing hydrogels. Biomaterials 2002;23:3957-3967.

02—Peppas NA, Bures P, Leobandung W, Ichikawa H. Hydrogels in pharmaceutical formulations. Eur J Pharm Biopharm 2000;50:27-46.

03—Park K, Park H. Biocompatibility issues of implantable drug delivery systems. Pharm Res 1996; 13(12):1770-1776.

04—Novick SJ, Dordick JS. Preparation of active and stable biocatalytic hydrogels for use in selective transformation. Chem Materials 1998;10(4):955-958.

05—Letourneur D, Machy D, Pelle A, Marcon-Bachari E, Dangelo G, Vogel M, Chaubert F, Michel J-B. Heparin and non-heparin-like dextrans differentially modulate endothelial cell proliferation: In vitro evaluation with soluble and crosslinked polysaccharide matrices. J Biomed Mater Res 2002;60:94-100.

06—Österberg E, Bergstrom K, Holmberg K, Schuman TP, Riggs JA, Burns NL, Van Alstine JM, Harris JM. Protein-rejecting ability of surface-bound dextran in end-on and side-on configurations: comparison to PEG. J Biomed Mat Res 1995;29:741-747.

07—Frazier RA, Matthijs G, Davies MC, Roberts CJ, Schacht E, Tendler SJB. Characterization of protein-resistant dextran monolayers. Biomaterials 2000;21:957-966.

08—Massia SP, Stark J. Immobilized RGD peptides on surface-grafted dextran promote biospecific cell attachment. J Biomed Mater Res 2001;56:390-399.

09—Wong WH, Mooney DJ. Synthesis and properties of biodegradable polymers used as synthetic matrices for tissue engineering. In Atala A, Mooney DJ, editors. Synthetic biodegradable polymer scaffolds. Birkhauser, Boston, 1997, p.50.

10—Lee KY, Mooney DJ. Hydrogels for tissue engineering. Chemical Reviews 2001; 101(7):1869-1879.

11—Hern DL, Hubbell JA. Incorporation of adhesion peptides into nonadhesive hydrogels useful for tissue resurfacing. J Biomed Mater Res 1998;39:266-276.

12—Mann BK, Gobin AS, Tsai AT, Schmedlen RH, West JL. Smooth muscle cell growth in photopolymerized hydrogels with cell adhesive and proteolytically degradable domains: synthetic ECM analogs for tissue engineering. Biomaterials 2001; 22:3045-3051.

13—De Groot CJ, Van Luyn MJA, Van Dijk-Wolthuis WNE, Cadee JA, Plantinga JA, Den Otter W, Hennink WE. In vitro biocompatibility of biodegradable dextran-based hydrogels tested with human fibroblasts. Biomaterials 2001;22:1197-1203.

14—Cadée JA, Luyn MJA, Brouwer LA, Plantinga JA, van Wachem PB, Groot CJ, Otter W, Hennink WE. In vivo biocompatibility of dextran-based hydrogels. J Biomed Mater Res 2000;50:397-404.

15—Anderson JM. In vivo biocompatibility of implantable delivery systems and biomaterials. Eur J Pharm Biopharm 1994;40(1):1-8.

16—Anderson JM. Inflammation, wound healing, and the foreign body reaction response. In: Ratner BD, Hoffman AS, Schoen FJ, Lemons JE, editors. Biomaterials Science. Academic Press, 1996. p 165-173.

17—ISO 10993-5: Biological evaluation of medical devices. Test for cytotoxicity: In vitro methods. Geneva, Switzerland: ISO; 1992.

18—Pizzoferato A, Ciapetti G, Stea S, Cenni E, Arciola CR, Granchi D, Savarino L. Cell culture methods for testing biocompatibility. Clinical Materials 1994; 15:173-190.

19—Bancroft JD, Stevens A. Theory and practice of histological techniques. Edinburg: Churchill Livingstone; 1996. p 99-211.

20—Mosmann T. Rapid colorimetric assay for cellular growth and survival: application to proliferation and cytotoxicity assays. J Imunol Methods 1983;65:55-63.

21—Moreau MF, Chappard D, Lesourd M, Montheard JP, Basle MF. Free radicals and side products released during methylmethacylate are cytotoxic for osteoblastic cells. J Biomed Mater Res 1998;40:124-131.

22—Black J. The inflammatory response. In Black J editor. Biological performance of materials. Marcel Dekker, New York, 1999, p.131.

23—Murch AR, Grounds MD, Marshall Calif., Papadimitriou JM. Direct evidence that inflammatory multinucleate giant cells form by fusion. J Pathol 1982;137:177-180.

24—Kuijpers AJ, Wachem PB, Luyn MJA, Plantinga JA, Engbers GHM, Krijgsveld J, Zaat SAJ, Dankert J, Feijen J. In vivo compatibility and degradation of crosslinked gelatin gels incorporated in knitted Dacron. J Biomed Mater Res 2000;51:136-145.

25—Kuijpers AJ, van Wachem PB, Luyn MJA, Brouwer LA, Engbers GHM, Krijgsveld J, Zaat SAJ, Dankert J, Feijen J. In vitro and in vivo evaluation of gelatin-chondroitin sulphate hydrogels for controlled release of antibacterial proteins. Biomaterials 2000;21:1763-1772.

26—Hooper KA, Macon N. Dak., Kohn J. Comparative histological evaluation of new tyrosine-derived polymers and poly(L-lactic acid) as a function of polymer degradation. J Biomed Mater Res 1998;41:443-454.

27—Bos RRM, Rozema FR, Boering G, Nijenhuis AJ, Pennings AJ, Verwey AB, Nieuwenhuis P, Jansen HWB. Degradation of and tissue reaction to biodegradable poly(L-lactide) for use as internal fixation of factures: a study in rats. Biomaterials 1991;12:32-36.

28—Nakamura T, Veda H, Tsuda T, Li Y—H, Kiyotani T, Inoue M, Matsumoto K, Sekine T, Yu L, Hyon S-H, Shimizu Y. Long-term implantation test and tumorigenicity of polyvinyl alcohol hydrogel plates. J Biomed Mater Res 2001;56:289-296.

29—Dziubla TD, Torjman MC, Joseph JI, Murphy-Tatum M, Lowman AM. Evaluation of porous networks of poly(2-hydroxyethylmethacrylate) as interfacial drug delivery devices. Biomaterials 2001;22:2893-2899.

30—Spector M, Lalor PA. In vivo assessment of tissue compatibility. In: Ratner BD, Hoffman AS, Schoen FJ, Lemons JE, editors. Biomaterials Science. Academic Press, 1996. p 220-228.

### Example 4

Enzymatic Synthesis of Inulin-Containing Hydrogels

Introduction

Enzymes are important catalysts in a wide range of reactions because of their catalytic rates, specificity and function under mild conditions². This is particularly evident in biotransformations catalyzed by hydrolases (e.g. proteases, lipases, etc) wherein a variety of nucleophiles act as substrates for enzyme-catalyzed acyl transfer in nearly anhydrous organic solvents³⁻⁷. This reaction breadth has been extended to polymer synthesis. In particular lipases have been shown to catalyze polytransesterification⁸⁻¹⁰ and ring opening polymerisation¹⁰⁻¹¹ in organic solvents, and proteases have been used for regioselective synthesis of sugar polyesters in the nonaqueous milieu¹²⁻¹³.

Although the traditional uses of enzymes for synthetic applications involve small molecules, the benefits of enzyme technology have been used in the modification of synthetic and natural polymers, particularly those that are soluble in organic solvents. For example, lipase from Candida antarctica was shown to catalyze the selective epoxidation of polybutadiene in organic solvents in the presence of hydrogen peroxide and catalytic quantities of acetic acid ¹⁴. Unlike synthetic polymers, polyhydroxylated compounds such polysaccharides are either sparingly soluble in only the most polar organic solvents or are incompletely insoluble. Nevertheless, enzymatic derivatization of polysaccharides have been performed either in non-polar organic solvents using insoluble polysaccharides with soluble¹⁵ and suspended enzymes¹⁶ or aqueous solution using insoluble polysaccharide and soluble enzyme¹⁷. The results obtained from non-aqueous enzymatic approaches showed that only surface chains could be enzymatically acylated whereas in aqueous enzymatic solutions it was impossible to control and characterize the reaction products. The rationale of the current work is to overcome these limitations.

Herein we report the first successful enzyme catalyzed modification of an organic solvent-soluble polysaccharide, inulin, using anhydrous DMF as the reaction medium. Inulin is composed by a mixture of oligomers and polymers containing 2 to 60 (or more) β-2,1 linked D-fructose molecules having a glucose unit as the initial residue¹⁸⁻¹⁹. While inulin is not digested in the upper gastrointestinal tract, it is hydrolyzed in the colon by intestinal flora. Thus, inulin-based materials may have use as drug delivery matrices for colonic targeting. These site-specific delivery systems can be used in the treatment of colonic disorders such as Crohn's disease or colon carcinoma's, reducing undesirable side effects caused by the therapeutic drugs used^{18,20}. For that purpose, we modified inulin with vinyl acrylate (Scheme 1) and then used free radical polymerization to yield crosslinked hydrogels. Hydrogels with different swelling and physical properties were obtained.

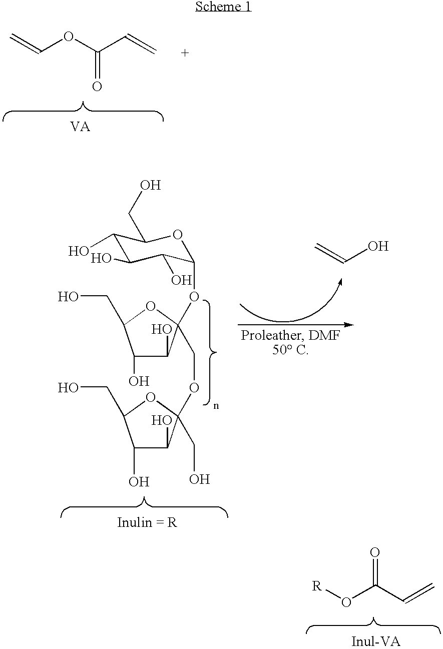

Experimental Section

Materials

Proleather FG-F, a protease from Bacillus sp. was a generous gift from Amano Enzyme Co. (Troy, Va., USA). Chicory inulin was purchased from Fluka Chemie AG (Buchs, Switzerland). Vinyl acrylate (VA), N,N-dimethylformamide (DMF), N,N,N′,N′-tetramethylenethylenediamine (TEMED), and ammonium persulfate (APS) were supplied by Aldrich (Milwauke, Wis., USA). DMF was dried with 3 Å molecular sieves at least overnight before use. Regenerated Cellulose dialysis tubes with a 1000 MWCO were purchased from Spectrum (CA, USA). All other chemicals and solvents used in this work were of the highest purity commercially available.

Methods

^{1H} and ¹³C NMR spectra were recorded on a Varian Unity spectrometer (Palo Alto, Calif.) at 300 MHz and 75 MHz, respectively. ¹H NMR spectra were recorded in D₂O (60-100 mg in 0.7 mL) using a pulse angle of 90° and a relaxation delay of 30 s. The water signal, used as reference line, was set at δ 4.75 ppm and was suppressed by irradiation during the relaxation delay. The number of scans in the spectra acquisition was 16. ¹³C NMR spectra were recorded in D₂O using a pulse angle of 30° and relaxation delay of 1 s. tert-Butanol (tb) was used as reference, which was set at δ 31.2 ppm versus tetramethylsilane. Generally, the number of scans was 16,000. Bi-dimensional spectra were recorded on a Varian Unity 500 MHz spectrometer (Palo Alto, Calif.). ¹H-¹H COSY spectra were collected as a 1,024×416 matrix covering a 2,500 Hz sweep width using 32 scans/increment. Before Fourier transformation, the matrix was zero filled to 2,048×2,048 and standard sine-bell weighting functions were applied in both dimensions. ¹H-¹³C HMQC spectra were collected as a 1,024×256 matrix covering sweep widths of 2,500 Hz and 11,500 Hz in the first and second dimensions, respectively. Before Fourier transformation, the matrix was zero-filled to 1,024×1,024 and standard gaussian weighting functions were applied in both dimensions.

FTIR spectra were recorded with a Nicolet Magna-IR 550 spectrometer (Madison, Wis.). The dry samples were powdered, mixed with KBr and pressed into pellets under reduced pressure. The FTIR spectra were obtained by recording 128 scans between 4000 and 450 cm⁻¹ with a resolution of 2 cm⁻¹.

The CP/MAS ¹³C NMR spectra were recorded on a 360 MHz Chemagnetics spectrometer (90.5 MHz) equipped with CP-MAS (cross-polarization magic-angle-spinning) accessories at 25° C. The sample (ca. 200-300 mg) was placed in a 7.5 mm Zirconia rotor (Chemagnetics PENCIL, Fort Collins, Colo.) and spun at 3 KHz. The contact time was 3 ms and a recycle time of 5 s was applied. The number of scans was set at 4,000. The ¹³C shifts were calibrated by substitution using external hexamethylbenzene.

Gel permeation chromatography (GPC) was performed with a Shimadzu LC-10 AT (Columbia, Md.) equipped with a Waters 410 refractive index detector (Milford, MA). The eluent was DMF at a flow rate of 0.5 mL/min. Waters 500 Å and 100 Å Ultrastyragel (7.5×300 mm), and Styragel HR 5E (4.6×300 mm) were installed in series to achieve effective separation of polymers. Calibration was made with polystyrene standards of narrow polydispersity in the molecular weight range from 762-44,000 Da. The GPC chromatograms were obtained from samples dissolved in DMF over a concentration range of 2.1-2.4% (w/v).

In some cases (as stated in the text), the determination of DS¹ was performed by titration based on a method described by Vervoort et al.²¹. Inulin derivatives (50 mg) were dissolved in 0.1 N NaOH (4 mL) and stirred for 72 h, at 20° C., to obtain alkaline hydrolysis of the ester. The molar consumption of NaOH was determined by back titration with 0.1 N HCl after adding 2 drops of phenolphthalein solution as indicator. Underivatized inulin was used as blank.

Pretreatment of Proleather FG-F and Inulin

Proleather FG-F was “pH-adjusted” in the presence of 20 mM phosphate buffer at pH 8.0, corresponding to the enzyme optimum pH according to the supplier. After flash-freezing in liquid nitrogen, the sample was lyophilized on a Labconco freeze drier (Labconco Corp., Kansas City, Mo.) for 48 h. Active site titration was performed before and after lyophilization according to the method of Schonbaum²² using N-transcinnamoylimidazole as the titrant. The percentage of active enzyme in the powder before and after lyophilization was 4.11±0.09% and 2.89±0.56% (average+SD, n=3), respectively.

Proleather FG-F thermally deactivated was prepared suspending the enzyme in 250 mL of 20 mM phosphate buffer pH 8.0 in a 500 mL round-bottomed flask fitted with a water-cooled condenser. The enzyme solution was refluxed for 5 h, after which was allowed to cool to room temperature and then lyophilized. The proteolytic activity of Proleather FG-F and its thermally deactivated form were determined with casein as the substrate. The enzyme solution (0.1 mL, 80 mg/mL) was added to the reaction media formed by a mixture of 1 mL of 0.1 M phosphate buffer pH 8.0 with 5 mL of 1.0% (w/v) casein solution. The mixture was incubated for 3 min at 37° C., with magnetic stirring (200 rpm), and a 0.5 mL aliquot was taken and added to an equal volume of 0.4 M trichloroacetic acid. The resulting precipitate was removed by centrifugation (5000 rpm, 2 min) after standing for 25 min at 25° C. The supernatant (0.5 mL) was placed in a test tube containing 5 mL of 0.4 M sodium carbonate and 0.5 mL of 5-fold diluted Folin's reagent. After thorough mixing, the solution was allowed to stand for 20 min at 37° C., and the absorbance measured spectrophotometrically at 660 nm. The absorbance values were then converted to equivalent tyrosine concentration using a tyrosine calibration curve. One unit of protease activity (U) is defined as quantity of enzyme needed to produce the amino acid equivalent of 1 □g of tyrosine per minute.

In some cases, as stated on the text, inulin was treated before reaction. Inulin (6.7%, w/v) was dissolved in 300 mL DMF and further centrifuged at 4000 rpm for 5 min. The supernatant was precipitated in 500 mL acetone and the precipitate dissolved in water and lyophilized for 48 h.

Enzymatic Synthesis of Inulin Ester Monomers

Preparative-scale reactions were performed in 60 mL of anhydrous DMF containing 0.017 M (6.7%, w/v) Inulin and variable concentrations of VA. The reaction mixtures were shaken (250 rpm) at 50° C. in a temperature-controlled New Brunswick Scientific C24 orbital shaker (Edison, N.J., USA) for 96 h. The reactions were terminated by the removal of the enzyme (which is insoluble in DMF) by centrifugation at 4,000 rpm for 10 min. The supernatants were precipitated in a 4-fold excess of acetone and further washed with the same solvent. The precipitate was subsequently dissolved in Milli-Q water and dialyzed using a regenerated cellulose dialysis tube with a 1000 MWCO for 2 days, at 4° C., against water. Afterwards, the aqueous solutions of Inul-VA were lyophilized for 48 h.

Time course reactions of Inulin ester synthesis by Proleather FG-F (10, 20 and 30 mg/mL) were performed independently in 15 mL of anhydrous DMF containing 0.017 M (6.7%, w/v) inulin and 0.204 M VA (molar ratio of vinyl monomer to inulin fructofuranoside residues x 100=50) at 250 rpm and 50° C. The purification of the products was performed as before.

Preparation of Inul-VA Gels

Inul-VA gels were obtained by free radical polymerization of aqueous solutions of Inul-VA as a function of DS and monomer concentration. Inul-VA (100, 200 or 400 mg) was dissolved in 0.9 mL of 0.2 M phosphate buffer pH 8.0 and the polymerization reaction performed in the Eppendorfe tubes (radius≅0.5 cm) was initiated by adding 50□L APS (80 mg/mL in 0.2 M phosphate buffer pH 8.0) and 50 □L TEMED solution (13.6% (v/v) in water; adjusted to pH 8.0 with 12 N HCl) for 24 h at 25° C. The gels were subsequently removed from the Eppendorf® tubes and immersed in 100 mL of 0.010 M citrate-phosphate buffer pH 7.0 for 5 days at 25° C., changing the buffer daily. At regular intervals, the swollen gels were removed, blotted with filter paper to remove surface water, weighed, and returned to the same container until weight stabilization was observed (5 days). The gels were then dried at room temperature, under vacuum, in the presence of phosphorous pentoxide, and weighed to determine the dried weight, W_{d}. The swelling ratio at equilibrium (SRE) was calculated according to Eq. 1.

$$SRE = \frac{{W}_{s} - {W}_{d}}{{W}_{d}} \qquad ( 1 )$$

The molecular weight between crosslinks (\overline{M}_{c}) was calculated with the model of Flory and Rehner²³, modified by Peppas et al.²⁴, according to Eq.3:

$$\frac{1}{{\overline{M}}_{c}} = \frac{2}{{\overline{M}}_{n}} - \frac{{( \frac{\overline{v}}{{V}_{1}} ) [ ln ( 1 - {v}_{2 , s} ) + {v}_{2 , s} + {{χ}_{1} ( {v}_{2 , s} )}^{2} ] [ 1 - \frac{1}{{χ}_{c}} {( \frac{{v}_{2 , s}}{{v}_{2 , r}} )}^{\frac{2}{3}} ]}^{3}}{{{v}_{2 , r} [ {( \frac{{v}_{2 , s}}{{v}_{2 , r}} )}^{\frac{1}{3}} - 0.5 ( \frac{{v}_{2 , s}}{{v}_{2 , r}} ) ] [ 1 + \frac{1}{{χ}_{c}} {( \frac{{v}_{2 , s}}{{v}_{2 , r}} )}^{\frac{1}{3}} ]}^{2}} \qquad ( 3 )$$

where \overline{M}ₙ is number average molecular weight of the inulin used (3,620 Da), v is the partial specific volume of inulin (0.601 cm³/g)²⁵, V₁ is the molar volume of water (18 cm³/g), χ₁ is the Flory polymer-solvent interaction parameter (0.473 taken from dextran/water system²⁶), χ_{c} is the number of links of the chain (χ_{c}=2\overline{M}_{c}/Mᵣ, where Mᵣ is the molecular weight of the inulin repeating unit, 162.14), v_{2,r} is the polymer fraction of the gel after gel formation and v_{2,r} is the polymer fraction at equilibrium swelling. V_{2,r} and v_{2,s} were calculated from the weight of the gels before exposure to the buffer solution and after equilibrium swelling, respectively, assuming volume additivity of water and inulin. The average mesh size, ξ, was calculated through the use of Eqs. 7 and 8²⁴:

\overline{r}₀²=Cₙχ_{c}b² (7)

ξ=v_{2,s}^{-1/3}(\overline{r}ₒ²)^{1/2} (8)

where \overline{r}ₒ² represents the average end-to-end subchain length (in Å) when the gel is unswollen, Cₙ is the polymer rigidity factor, assumed to be 8.9 by analogy to polar poly(vinyl alcohol)²⁷ and b is the characteristic bond length of the polymer backbone (=1.54 Å, corresponding to the C—C bond length). The crosslinking density, ρx, was determined from Eq. 14 (this expression does not take into account the effect of physical entanglements)²⁴:

$${ρ}_{x} = \frac{1}{{\overline{vM}}_{c}} \qquad ( 14 )$$

The theoretical crosslinking density was also calculated from eq. 14, nevertheless the theoretical number average molecular weight between crosslinks was calculated from Eq. 15.

$${\overline{M}}_{c , theor} = \frac{{M}_{r} \times 100}{DS} \qquad ( 15 )$$

Results and Discussion

Synthesis of VA Derivatized Inulin

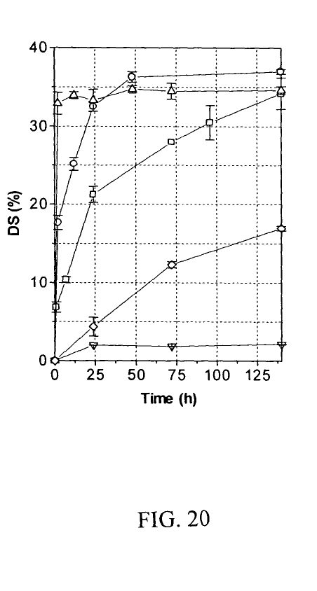

Recently we found that Proleather FG-F enzyme was able to acylate inulin with divinyl adipate in DMF (unpublished results)²⁸, from a range of eleven enzymes (including proteases and lipases), and therefore was chosen in this present work. VA was selected as an activated acrylate acyl donor that is known for its high reactivity in enzyme-catalyzed transesterification reactions²⁹. The time-course reaction of inulin with VA, at 50° C., at increasing concentrations of enzyme is shown in FIG. 20. As expected, the initial reaction rate increases as a function of the enzyme concentration. Quantitative measurement of acrylate incorporation onto the inulin backbone was possible in 2 h, when 30 mg/mL of Proleather was used. Further analysis of FIG. 20 reveals that DS (corresponding to a conversion³⁰ of ca. 70%) of Inul-VA is practically unchanged after 50 h, for Proleather FG-F concentrations of 20 and 30 mg/mL, indicating that all the reactive sites on inulin have acrylate functionalities attached.

In parallel to these time-course reactions with Proleather, control reactions in the absence or with thermally deactivated enzyme were also carried out (FIG. 20). In the absence of added active enzyme no significant (DS ca. 2%) inulin derivatization occurred in 140 h of reaction. However, unexpectedly, the addition of Proleather thermally deactivated for 5 h at 100° C. did catalyze a noticeable transesterification of inulin with VA to give a DS of ca. 17% after 140 h. To assess whether this conversion could be a result of nonspecific reactions due to nucleophilic functionalities in the protein preparation, or due to a true intrinsic catalytic residual enzymatic activity, the proteolytic activity of the heat-treated enzyme preparation was measured using casein as substrate. It was found that active Proleather FG-F and its deactivated preparation had activity values of 104.9+7.9 and 10.3+0.3 U per mL of enzyme solution, respectively, and this corresponded to a similar ratio of reactivities using 20 mg/mL Proleather on inulin. Thus, it may be concluded that the residual inulin activity present in the heat-treated Proleather was due to intrinsic activity, and this further suggests that the enzyme is thermostable. Furthermore, we found that some enzymes did not present any activity on the polytransesterification reaction of inulin with divinyl adipate²⁸, further corroborating the absence of nonspecific reactions due to their external aminoacids (not involving the catalytic site). Based on these results, we performed preparative-scale synthesis of inulin esters for 96 h in the presence of 20 mg/mL Proleather at 50° C.

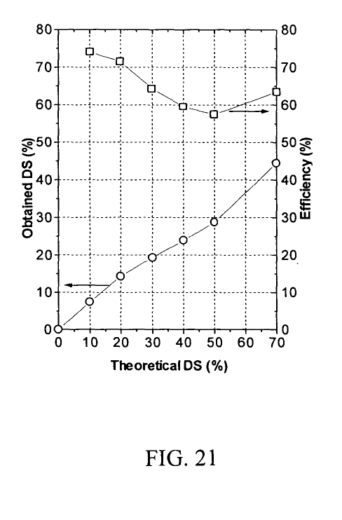

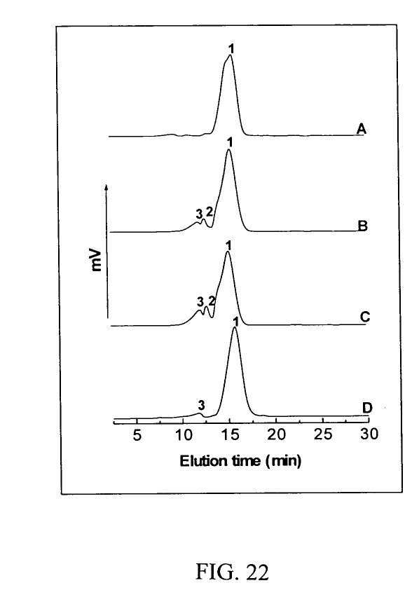

FIG. 21 shows the relationship between the molar ratio of VA to inulin fructose units in the reaction mixture (theoretical DS) and the degree of substitution of the products, as determined by ¹H NMR (obtained DS). From these results, Inul-VA can be obtained with different DS, ranging the concentration of the acyl donors. The efficiency of the coupling reaction (calculated as the ratio of the obtained DS to the theoretical DS) was above 57.4%.

The two-step purification procedure adopted in this work, based in a precipitation with acetone followed by dialysis against water, revealed to be an efficient way to obtain ester products with no impurities, as detected by ¹H NMR spectroscopy (Isolated yield of 44-51%, except for Inul-VA DS 44.4%, which had an isolated yield of 27.7%). Yet due to the easy removal of Proleather enzyme from the reaction mixture (insoluble in DMF), the purification protocol is faster than the one presented by Vervoort et al.²¹ which employed an extensive dialysis process for 10 days to remove the catalyst 4-dimethylaminopyridine in the metracrylation reaction of inulin.

GPC Analysis

GPC analysis of Inulin and Inul-VA derivatives showed different elution profiles. Representative chromatograms of these polymers are presented in FIG. 22. Inulin GPC-chromatogram shows a single peak corresponding a Mₙ of 3,620 Da (M_{w}/Mₙ=1.2) and an average degree of polymerization of 25. The same profile was not observed for Inul-VA samples with different DS's. Chromatograms B and C present besides a major peak (BI or C1) two other minor peaks (B2 and B3, Mₙ of 13,450 Da and 35,160 Da, respectively; C2 and C3, Mₙ of 12,180 Da and 31,640 Da, respectively). The major inulin peak in those samples is shifted to higher molecular weight from Mₙ 4,100 Da to Mₙ 4,440 Da when DS values increase. This is likely due to introduction of acrylate groups in the inulin backbone. The minor peaks in chromatogram B and C (representing 12.4% and 19.7% of the sample, respectively) could be ascribed to enzyme contaminants from the crude enzyme preparation. However, GPC analysis of Proleather FG-F dissolved in DMF (data not shown) showed no contribution of the biocatalyst in the appearance of that minor peaks. Another possible explanation was the presence of high molecular weight polymers in the inulin which were not originally soluble in DMF but soluble after derivatization with VA. It is noteworthy to mention that traces of original inulin were not completely soluble in DMF and therefore its GPC analysis (chromatogram A), using DMF as eluent, shows just the soluble moiety. As the transesterification reaction proceeds, high molecular weight polymers may become soluble in DMF due to modification with the relatively hydrophobic VA. This was further verified removing the DMF-insoluble fraction of inulin by centrifugation and precipitating the supernatant in acetone. The precipitated-inulin was reacted with VA in the presence of Proleather and the reaction product characterized by GPC. The GPC-chromatogram obtained (D) shows that one of the minor peaks is totally removed (corresponding to B2 and C2) and the other one is partially removed (corresponding to B3 and C3). Even if the removal of minor peaks was not complete the results suggest that those peaks are related to high molecular weight polymers. Similar results showing small amounts of high molecular weight polymers on Inulin were described by Verraest et al.³¹ by GPC analysis using 0.1 M NaNO₃ as eluent.

Characterization by NMR Spectroscopy

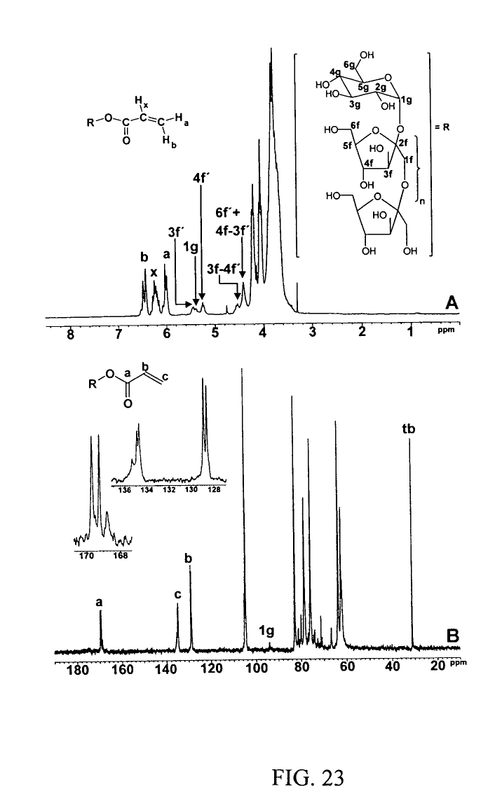

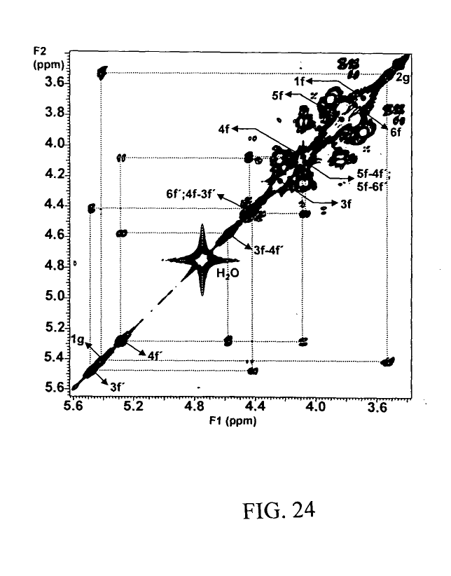

The structure of Inul-VA was analyzed by NMR spectroscopy. FIG. 23 displays ¹H (A) and ¹³C (B) NMR spectra of Inul-VA. In the ¹H NMR spectrum (spectrum A) the intense peaks between δ 3.38-4.23 ppm are attributed to protons of unreacted inulin, including the anomeric proton at δ 5.42 ppm belonging to the D-glucopyranosyl units. The assignments of each proton signals are clearly shown in the ¹H-¹H COSY displayed in FIG. 5. Furthermore, from the ¹H NMR spectrum of Inul-VA the signals from the acrylate groups are observed at δ 6.4 ppm (H_{B}, ³J_{BX}=17.21 HZ,²J_{BA}=1.47 Hz), δ6.2 ppm (H_{X}, ³J_{XA}=10.38 Hz,³J_{XB}=17.21 Hz),δ6.0 ppm (H_{A}, ³J_{AX}=10.38 Hz,²J_{AB}=1.47 Hz).

The formation of Inul-VA is also confirmed by its ¹³C NMR spectrum (FIG. 23, spectrum B). The fructofuranosyl and acrylate carbons are displayed in the range of 62.0-105.2 ppm and 128.6-169.7 ppm, respectively. Except for carbon α (denoted as b in FIG. 23) of the double bond (duplicate: 128.9 and 128.6 ppm), all other signals are in triplicate (Cₐ:169.7, 169.2 and 168.8 ppm; C_{c},:135.3, 134.9 and 134.7). This indicates the presence of three different positional isomers in the Inul-VA product.

The ester positions on the fructofuranosyl ring were assigned based on the additional signals presented in ¹³C NMR spectrum (FIG. 23) of Inul-VA ranging from δ 105.2 to 62.0 ppm. According to the literature³² chemical shifts of acylated carbons suffer a downfield shift and the respective adjacent carbons a concomitant upfield shift. The chemical shifts of the other carbon atoms are hardly affected. As shown in the ¹³C NMR spectrum there is no upfield shift of C-2 carbons, which appears to indicate no positional isomer at position 3. Therefore, the two acylated isomers in the main inulin backbone are at positions 6 and 4 in the fructofuranosyl ring. The respective ¹³C-NMR assignments are presented in Table 8.

Table 8—¹³C NMR assignments of the fructofuranosyl ring carbons (δ, ppm) on Inul-VA with DS 28.7%.

| | | Inul-VA | | | | | |
|---|---|---|---|---|---|---|---|
| | Inulin | 6-substituted | | 4-substituted | | 3-substituted | |
| | Obs. | Obs. | | Obs. | | Obs. | |
| Carbon | signal | signal | Δδ | signal | Δδ | signal | Δδ |
| 1 | 62.4 | 62.1 | −0.3 | 61.9 | −0.5 | 61.7 | −0.7 |
| 2 | 104.8 | 104.7 | −0.1 | 105.0 | +0.2 | 105.3 | +0.5 |
| 3 | 78.6 | 78.4 | −0.2 | 76.9 | −1.7 | 80.6 | +2.0 |
| 4 | 75.8 | 76.1 | +0.3 | 78.2 | +2.4 | 74.6 | −1.2 |
| 5 | 82.6 | 80.0 | −2.6 | 81.2 | −1.4 | 82.4 | −0.2 |
| 6 | 63.7 | 66.8 | +3.1 | 63.5 | −0.2 | 63.9 | +0.2 |

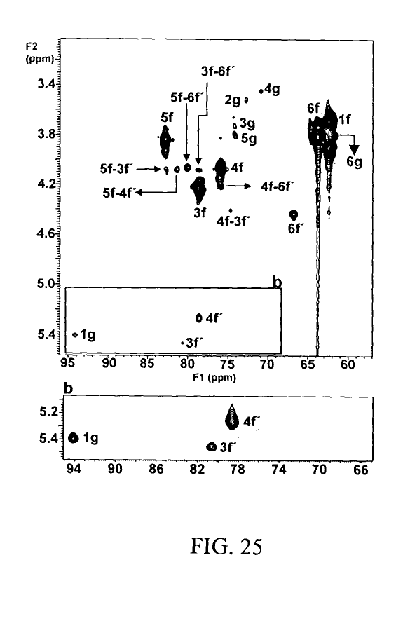

An ¹H-¹³C HMQC NMR experiment was acquired in order to correlate those ¹³C signals with ¹H signals (FIG. 25). In this spectrum the ¹³C peaks at δ 66.8 ppm (modification at 6 position) and δ 78.2 ppm (modification at 4-position) are correlated with ¹H signals at δ 4.41 ppm (6f′) and δ 5.24 ppm (4f′), respectively. From ¹H-¹H COSY (FIG. 25) the signal at δ 5.24 ppm has two cross-peaks at 4.58 ppm and 4.08 ppm corresponding to the vicinal protons at positions 3 (denoted as 3f-4f′) and 5 (5f-4f′), while the signal at δ 4.41 ppm has a single correlation with a peak at 4.09 ppm corresponding to a vicinal proton at position 5 (5f-6f′). However, still remaining is the assignment of the third isomer. In the ¹H NMR spectrum of Inul-VA there is a small signal at δ 5.45 ppm that overlaps with the D-glucopyranosyl anomeric proton at 5.42 ppm, the latter correlating in the ¹H-¹³C HMQC spectrum with a ¹³C signal at δ 80.6 ppm. This signal corresponds to an acylated carbon at position 3 (Table I). This is further confirmed by the ¹H-¹H COSY spectrum, which shows a single cross-peak for this signal at 4.42 ppm corresponding to a vicinal proton at position 4 (4f-3f′). Interestingly, as previously mentioned, there is no upfield shift in the ¹³C NMR spectrum corresponding to the C-2 position, as would be expected according to the literature³². This might be due to the absence of protons attached to that carbon, thereby mitigating the observed upfield shift.

Based on the ¹H NMR assignments, the DS was determined using Eq. 9:

DS=(7x/y)*100 (9)

where x is the average integral of the protons from vinyl group (δ 6.0-6.4 ppm) and y is the integral of all inulin protons.

Distribution of the Acrylate Substituents

Based on the ¹H NMR assignments (FIG. 23, spectrum A) the relative DS (DSᵢ) of modified individual hydroxyl groups attached to the C-3, C-4 and C-6 carbons have been estimated from the following equations:

DS₃=[x(DS)]/y (10)

DS₄=[z(DS)]/y (11)

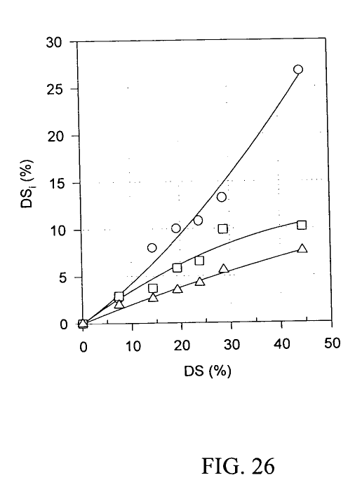

DS₆=[(x+z)−DS]/y (12)

where x and z are the integral of the proton signals at δ 5.45 ppm and δ 5.24 ppm, respectively; y is the average integral of the protons from vinyl group (δ 6.0-6.4 ppm); and DS is the total degree of substitution calculated from Eq. 9. Using these equations the distribution of substituents in Inul-VA samples with different DS was calculated and presented in FIG. 26. The results obtained indicate that the reactivity's of hydroxyl groups towards acylation reaction decreases in the order C6f>C4f>C3f, where f indicates the fructosyl moiety. Since ¹H-¹H COSY clearly indicates that all fructose units substituted are mono-substituted (no cross-peaks are shared by the three positional isomers), the relative reactivities of the hydroxyl groups are not influenced by substitution of other positions in the unit. Furthermore, as shown in FIG. 26, the relative reactivities of the hydroxyl groups are independent of the DS, which demonstrates that the acylation of a particular hydroxyl group follows an independent trend.

The substitution pattern achieved in the Inul-VA samples with different DS shows the expected enzyme's preference for primary hydroxyl groups, and agreeing with results described in the literature showing that enzymatic acylation of small nucleophiles, including sugars occuring preferentially at the primary hydroxyl groups³⁻⁵. However, there is also the derivatization of secondary hydroxyl groups at positions 3 and 4, albeit to a lesser degree. Another interesting remark from the enzymatic derivatization of inulin is the monoester formation per fructofuranoside residue which seems to be distinct from the chemical derivatization of inulin reported in the literature. In fact, di-substituted fructofuranoside residues were founded in the inulin backbone when molar ratio's of acylating agent and inulin reported in this work were 33 used in carboxymethyl and cyanoethylation reactions

Preparation and Characterization of Inul-VA Gels

The acrylate groups in Inul-VA were polymerized to form a cross-linked network. The polymerization proceeded quickly, and within ca. 5 min the solution started to gell. The minimal DS necessary to gell 40, 20 and 10% (w/v) Inul-VA solutions in the presence of a free radical initiator was 7.4, 14.3 and 23.8%, respectively.

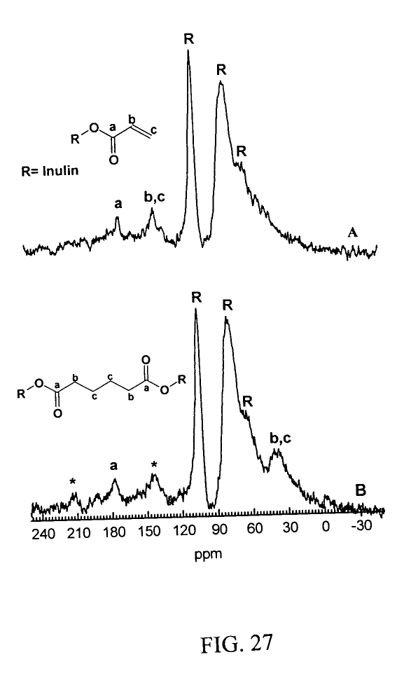

To follow the polymerization reaction, ¹³CP MAS NMR spectroscopy was performed (more conventional FTIR spectroscopy could not be used due to the overlapping inulin bands with vinyl monomer bands at 1635 cm⁻¹ (stretching of C═C bond) and ca. 811 cm⁻¹ (twisting of CH bond, from vinyl group) (data not shown)). ¹³CP MAS NMR spectra of unreacted Inul-VA and after 24 h of polymerization is displayed in FIG. 27. Upon polymerization the carbon from carbonyl group is shifted from δ 169.3 pm to δ 177.8 ppm as a result of a hybridization change in the adjacent carbons (vinyl carbons) from sp² to sp³. Furthermore, the tertiary methine carbon from the vinyl group shifts from δ 139.1 ppm to δ 39.8 ppm, while the secondary methylene carbon at 131.7 ppm is shifted to around 55 ppm²¹, overlapped by inulin carbons. Even if the degree of conversion of the acrylate groups could not be quantitatively determined by CPMAS, the results confirm the polymerization of vinyl monomers attached to inulin.

The determination of structural properties of crosslinked structures is crucial for gel characterization. The determination of the polymer volume fraction before and after swelling allows the calculation of the molecular weight between crosslinks, \overline{M}_{c}, according to Eq. 3. This equation developed by Peppas et al.²⁴ describes the swelling of a highly crosslinked, moderately swollen polymeric network. This approach takes into account the small average chain length between crosslinks (fewer than 100 repeating units), which deviates from a Gaussian distribution²⁴. Another critical parameter of gels is their average mesh size,ξ, which is important to assess the transport properties of solutes. ξ was calculated from \overline{M}_{c} by Eqs 7 and 8.

SR, \overline{M}_{c} and ξ from Inul-VA gels were determined as a function of monomer concentration and degree of substitution of the monomers, and are given in Table 9.

TABLE 9
Network properties of Inul-VA gels as a function of the initial monomer
concentration and the DS.

| | W₀^{a} | DS^{b} | | | \overline{M}_{c}^{e} | ξ^{f} |
|---|---|---|---|---|---|---|
| Gel | (%, w/v) | (%) | SRE^{c} | □_{2,s}^{d} | (g/mol) | (A⁰) |
| 1 | 10 | 28.7 | 19.80 ± 0.09 | 0.048 ± 0.001 | 1640.6 ± 1.6 | 56.83 ± 0.09 |
| 2 | 20 | 19.3 | 12.03 ± 0.30 | 0.077 ± 0.002 | 1535.2 ± 12.4 | 47.04 ± 0.36 |
| 3 | 20 | 23.8 | 8.04 ± 0.21 | 0.111 ± 0.003 | 1268.6 ± 20.8 | 37.86 ± 0.29 |
| 4 | 20 | 28.7 | 5.64 ± 0.01 | 0.151 ± 0.001 | 956.4 ± 1.1 | 29.65 ± 0.01 |
| 5 | 40 | 14.3 | 6.05 ± 0.13 | 0.142 ± 0.003 | 1209.9 ± 18.1 | 34.03 ± 0.21 |
| 6 | 40 | 19.3 | 3.59 ± 0.06 | 0.218 ± 0.003 | 769.3 ± 13.8 | 23.53 ± 0.11 |
| 7 | 40 | 23.8 | 3.31 ± 0.11 | 0.232 ± 0.006 | 709.4 ± 24.5 | 22.12 ± 0.19 |
| 8 | 40 | 28.7 | 2.73 ± 0.15 | 0.269 ± 0.010 | 583.7 ± 31.2 | 19.11 ± 0.25 |
| 9 | 40 | 44.4 | 2.71 ± 0.03 | 0.270 ± 0.002 | 579.3 ± 6.7 | 19.00 ± 0.05 |

^{a}Initial monomer concentration.
^{b}Degree of substitution, i.e., the amount of vinyl groups per 100 fructose units, determined by ¹H NMR.
^{c}Swelling ratio at equilibrium (average ± SD, n = 3).
^{d}Polymer fraction at equilibrium swelling (average ± SD, n = 3).
^{e}Molecular weight between crosslinks (average ± SD, n = 3).
^{f}Average mesh size (average ± SD n = 3).

SR, \overline{M}_{c} and ξ decrease as the monomer concentration increases from 10 to 40% (w/v), while maintaing a constant DS of Inul-VA monomer (28.7%). In this case, SR and ξ decreases from 19.80 to 2.73 and from 56.83 Å to 19.11 Å, respectively. These results can be explained by the increasing number of intermolecular crosslinks formed at higher monomer (Inul-VA) concentrations, which restricts network expansion upon swelling. In Inul-VA gels inter- and intramolecular crosslinking is likely to occur (besides some acrylates that do not react³⁴); however, intermolecular crosslinking is promoted by concentrated solutions, whereas intramolecular crosslink is predominantly formed from dilute solutions³⁴. Since intramolecular crosslinks do not contribute to the elastic effectiveness of the network, the SR is determined by the intermolecular crosslinks³⁵. The decreased \overline{M}_{c}, and therefore ξ, as monomer concentration increases can also be explained in the same way as SR, by the increasing of intermolecular crosslinking. Assuming 100% conversion of vinyl bonds on Inul-VA (DS 28.7%) by intermolecular crosslinking formation, a theoretical \overline{M}_{c} value (see eq.6) of 564 g/mol ([100*162.14]/DS) is expected. From table 9, changing initial Inul-VA concentration from 10% to 40% corresponds to a decreasing on \overline{M}_{c} from 1640.6 to 583.7 g/mol which further confirms the increasing number of intermolecular crosslinks. Finally, and because the \overline{M}_{c} calculated on the upper case was slightly lower than the theoretical value, there is a contribution of polymer chain entanglements at higher initial monomer concentration. These entanglements act as additional crosslinks to reduce SR, \overline{M}_{c} and ξ. Indeed, it has been reported²⁴ that \overline{M}_{c} calculated by swelling measurements is lower than that obtained from tensile strength measurements due to the effect of physical entanglements.

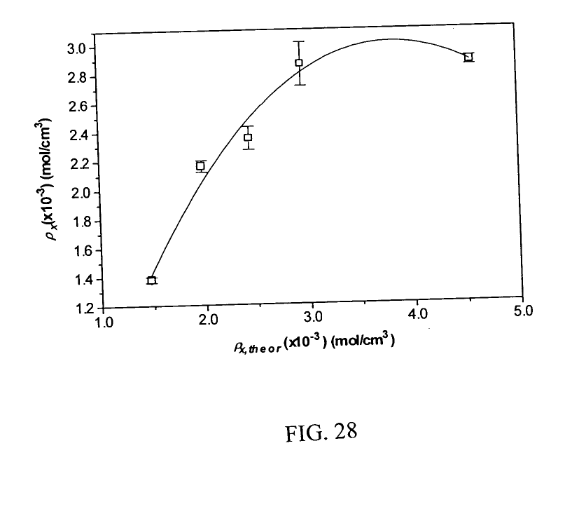

As summarized in Table 9, maintaining a constant starting monomer concentration (40%, w/v), the SR, \overline{M}_{c}, and ξ decrease as monomer DS increases in the starting polymerizing solutions. This agrees with the increasing number of intermolecular crosslinks formed by the high number of vinyl groups attached to inulin. From FIG. 28, the experimental crosslinking density (ρₓ) is shown to be slightly higher than the theoretical crosslinking density (ρ_{x,theor}) upon ρ_{x,theor} of ca. 2.0×10-3 mol cm⁻³ (corresponding to gels 5 and 6) due to the physical entanglements. However, for ρ_{x,theor} values higher than 2.0×10⁻³ mol cm⁻³ (corresponding to gels 7, 8 and 9) the ρₓ values are lower than ρ_{x,theor}, which indicates that some vinyl groups did not react and therefore do not contribute to effective crosslinking.

The calculation of both □_{2,s} and □ for Inul-VA gels allows us to establish a correlation among these parameters. Since □_{2,s} can be easily determined by the swelling of the crosslinked network a correlation between □_{2,s} and □it will be important to determine which solute could be applied in these gels. According to deGennes³⁶ for semidilute polymer solutions (□_{2,s}≦0.01) □ is related to □_{2,s} by a power-law exponent of −0.75. At high polymer concentrations (□_{2,s}>0.01) power-law exponents of −0.5³⁷ and −1²⁷ were reported on the literature. Inul-VA gels were analyzed using Eq. 13, using a linear regression with a predetermined exponent n:

ξ=k₁+k₂v_{2,s}ⁿ (13)

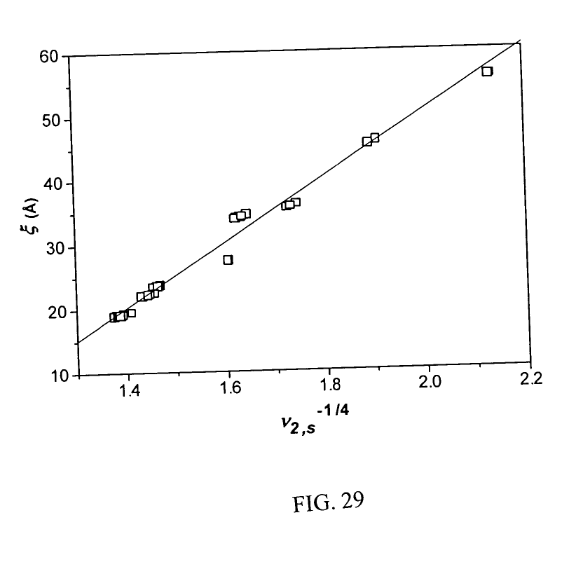

For the data of Inul-VA hydrogels with □_{2,s} between 0.048 and 0.270, eq. 13 with n=−0.25, k₁=−50.4, k₂=50.4 gives a good correlation (r²=0.9923) (FIG. 29). Other power-law exponents were fitted (n=−1, −0.75 and −0.5); however, the correlation coefficients were lower than the obtained for n=−0.25. It is noteworthy that all hydrogel samples were prepared from different initial polymer and crosslinker concentrations, and the correlation obtained seems to extent to all gels prepared. The average mesh size range achieved for Inul-VA gels suggest that they may have applications in controlled release of compounds with low molecular weight (high crosslinked gels) or macromolecular compounds such as proteins (low crosslinked gels). In this case, globular proteins with a molecular weight of 30,000 Da which have a diameter³⁸ of ≅42 Å could be administered through gels 1 and 2 (Table 9).

Conclusions

This work reports the first successful enzyme catalyzed modification of a soluble-polysaccharide, in this case inulin, in anhydrous DMF. Incorporation of vinyl groups in the inulin backbone was accomplished by transesterification of inulin with vinyl acrylate catalyzed by Proleather. The efficiency of the transesterification reaction and the isolated yield were above 57.4 and 44.0%, respectively. The structure of inulin esters revealed one predominant positional isomer in the fructofuranoside residue at the 6 position and two minor isomers at the 3 and 4 positions. Upon free radical polymerization of aqueous solutions of Inul-VA, hydrogels were obtained, which may be used as colon-specific drug delivery systems. The calculated values of

\overline{M}_{c} varied between 579.3 and 1640.6 g/mol, which corresponded to an average mesh size of 19.00 Å to 56.83 Å. A correlation was established between ξ and □_{2,s}. The exponent of this correlation was found to be −0.25 and it allows to define which drug may be loaded in the inulin gels by the simple determination of their swelling characteristics.

The enzymatic process described herein can be envisioned as a new method for the modification of polymers in nonaqueous media. We are presently using this enzymatic approach to derivatize other polysaccharides and hydroxylated polymers.

### REFERENCES AND NOTES FOR EXAMPLE 4

01—The degree of substitution (DS) is defined as the amount of acrylate groups per 100 inulin fructofuranoside residues.

02—Schmid, A.; Dordick, J. S.; Hauer, B.; Kiener, A.; Wubbolts, M.; Witholt, B. Nature, 2001, 409 (11), 258-267.

03—Patil, D. R.; Dordick, J. S.; Rethwisch, D. G. Macromolecules 1991, 24(11), 3462-3463.

04—Rich, J. O.; Bedell, B. A.; Dordick, J. S. Biotechnol. Bioeng. 1995, 45, 426-434.

05—Riva, S.; Chopineau, J.; Kieboom, A. P. G.; Klibanov, A. M. J. Am. Chem. Soc. 1988, 110, 584-589.

06—Carrea, G.; Riva, S. Angew. Chem. Int. Ed. 2000, 39, 2226-2254.

07—Koeller, K. M.; Wong, C.-H. Nature 2001, 409, 232-240.

08—Chaudhary, A. K.; Beckman, E:J.; Russell, A. J. Biotechnol. Bioeng. 1997, 55(1), 227-239.

09—Chaudhary, A. K.; Beckman, E:J.; Russell, A. J. J. Am. Chem. Soc. 1995, 117, 3728-3733.

10-Al-Azemi, T.; Bisht, K. S. Macromolecules 1999, 32, 6536-6540.

11-Bisht, K. S.; Svirkin, Y. Y.; Henderson, L. A.; Gross, R. A. Macromolecules 1997, 30, 7735-7742.

12-Patil, D. R.; Rethwisch, D. G.; Dordick, J. S. Biotechnol. Bioeng. 1991, 37, 639-646.

13—Park, O.-J.; Kim, D.-Y.; Dordick, J. S. Biotechnol. Bioeng. 2000, 70(2), 208-216.

14—Jarvie, A. W. P.; Overton, N.; Pourcain, C. B. S. J. Chem. Soc. Perkin Trans. 1, 1999, 2171-2176.

15—Bruno, F. F.; Akkara, J. A.; Ayyagari, M.; Kaplan, D. L.; Gross, R.; Swift, G.; Dordick, J. S. Macromolecules 1995, 28, 8881-8883.

16—Li, J.; Xie, W.; Cheng, H. N.; Nickol, R. G.; Wang, P. G. Macromolecules 1999, 32, 2789-2792.

17—Kumar, G.; Bristow, J. F.; Smith, P. J.; Payne, G. F. Polymer 2000, 41, 2157-2168.

18—Verraest, D. L. Ph.D. Thesis, Delft University Press, Netherlands, 1997.

19—Roberfroid, M. B.; Van Loo, J. A. E.; Gibson, G. R. J. Nutr. 1998, 128, 11-19.

20—Niness, K. R. J. Nutr. 1999, 129S (7S), 1402S-1406S.

21—Vervoort, L.; Van den Mooter, G.; Augustijns, P.; Busson, R.; Toppet, S.; Kinget, R. Pharm. Res. 1997, 14 (12), 1730-1737.

22—Schonbaum, G. R.; Zemer, B.; Bender, M. L. J. Biol. Chem. 1961, 236, 2930-2935.

23—Flory, P. J.; Rehner, R. J. Chem. Phys. 1943, 11, 521-526.

24—Peppas, N. A.; Moynihan, H. J.; Lucht, L. M. J. Biomed. Mater. Res. 1985, 19, 397-411.

25—Azis, B. H.; Chin, B.; Deacon, M. P.; Harding, S. E.; Pavlov, G. M. Carbohydr. Polym. 1999, 38(3), 231-234.

26—Gekko, K. In ACS Symposium Series Vol.150; Brant, D. A., Ed.; American Chemical Society: Washington, D.C., 1981; p.415-438.

27—Canal, T.; Peppas, N. A. J. Biomed. Mater. Res. 1989, 23, 1183-1193.

28—Ferreira, L.; Gil, M. H.; Dordick, J. S. (manuscript in preparation).

29—Uyama, H.; Kobayashi, S. Chem. Lett. 1994, 1687-1690.

30—The conversion is defined as the percentage of acrylate groups attached to inulin taking into account the initial molar ratio of VA to inulin fructose units in the reaction mixture (×100).

31—Verraest, D. L.; Peters, J. A.; Batelaan, J. G.; Van Bekkum, H. Carbohydr. Res. 1995, 271, 101-107.

32—Yoshimoto, K.; Itatani, Y.; Tsuda, Y. Chem. Pharm. Bull. 1980, 28(7), 2065-2076.

33—Verraest, D. L.; Peters, J. A.; Kuzee, H. C.; Raaijmakers, H. W. C.; van Bekkum, H. Carbohydr. Res. 1997, 302, 203-212.

34—Van Dijk-Wolthuis, W. N. E.; Franssen, O.; Talsma, H.; Steenbergen, M. J.; Kettene-van den Bosch, J. J.; Hennink, W. E. Macromolecules 1995, 28, 6317-6322.

35—Hennink, W. E.; Talsma, H.; Borchert, J. C. H.; De Smedt, S. C. Demeester, J. J. Contr. Release 1996, 39, 47-55.

36—deGennes, P. G. Macromolecules 1976, 9, 587-593.

37—Muthukumar, M.; Edwards, S. F. Polymer 1982, 23,345-348.

38—Chapman, J. D.; Hultin, H. O. Biotechnol. Bioeng. 1975, 17, 1783-1795.

### Example 5

Biocatalytic Polytransesterification of Inulin with Divinylapidate

Enzyme-catalyzed polytransesterification reaction of inulin, a natural polysaccharide, with divinyladipate (DVA) in dimethylformamide (DMF) was investigated. To our knowledge, this is the first report dealing with enzyme-catalyzed polycondensation reactions using a polysaccharide as starting material. Of eleven proteases and lipases screened, Proleather FG-F from Bacillus subtilis (EC 3.4.21.62) yielded the highest conversion of inulin to give polyesters with Mₙ greater than 20,000 Da (corresponding to the polymerization of five units of inulin consisting of a total of ca. 110 units of fructose), according to gel permeation chromatography. The structure of the polyester was established by ¹H, ¹³C, ¹H-¹H COSY, and ¹H-¹³C HMQC NMR spectroscopy. The polyester consisted of DVA molecules attached to the inulin backbone mainly through both ester groups of the DVA, but also through one of the ester moieties of the DVA to yield the crosslinked inulin molecules bridged by adipate and terminal vinyladipate groups, respectively. The polytransesterification reaction occurred mainly at the 6-position on the fructofuranoside ring, and to a lesser degree at the 3- and 4-positions. Thus, the enzymatic reaction was largely regioselective. The effect of the DVA and enzyme concentration, and reaction time was also evaluated.

Enzymatic synthesis of polymers has attracted significant attention in recent years¹ due to high inherent selectivity under mild reaction conditions. A wide range of polymers have been synthesized using purely enzymatic means, including polyphenols², polyesters³, and polycarbonates⁴. Although the vast majority of polymers have been prepared from rather simple monomers, enzymes offer the opportunity to incorporate complex polyfunctional compounds, such as sugars and polysaccharides into polymer backbones⁵, thereby extending the synthetic repertoire of polymer chemistry.

In the current work, we report the enzyme-catalyzed polytransesterification of inulin with divinyladipate (DVA) in DMF to produce inulin polyesters. Inulin is composed by a mixture of oligomers and polymers containing 2 to 60 (or more) β2-1 linked D-fructose molecules having a glucose unit as the initial residue⁶. Six proteases and five lipases, all commercially available, were tested for their abilities to catalyze the polytransesterification of inulin with DVA in anhydrous DMF (Scheme 2), at 50° C., for 72 h (Table 10)⁷.

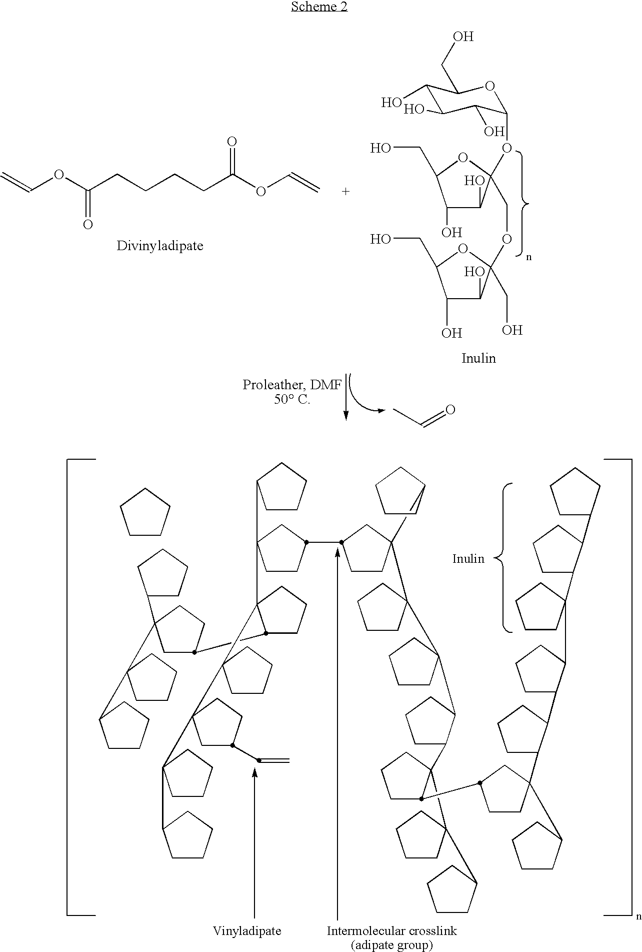

TABLE 10
Enzyme screening for the polytransesterification
reaction of inulin with DVA.

| | | | Conv. | | M_{w}/ |
|---|---|---|---|---|---|
| Entry | Enzyme | Origin | (%)^{c} | Mₙ | Mₙ |
| 1 | Proleather FG-F^{a} | Bacillus subtilis | 56.8 | 14,310 | 2.5 |
| 2 | Protease A^{a} | Aspergillus oryzae | 11.2 | 6,130 | 1.8 |
| 3 | Protease N^{a} | Bacillus subtilis | 8.4 | 5,420 | 1.9 |
| 4 | Protease P^{a} | Aspergillus melleus | 17.2 | 7,590 | 2.1 |
| 5 | Protease S^{a} | Bacillus | 3.8 | 5,560 | 1.1 |
| | | stearothermophilus | | | |
| 6 | Protease | Bacillus | 6.2 | 5,780 | 2.3 |
| | Subtilisin | licheniformis | | | |
| | Carlsberg^{b} | | | | |
| 7 | Lipase A^{a} | Aspergillus niger | 14.4 | 6,640 | 2.2 |
| 8 | Lipase AY^{a} | Candida rugosa | 36.4 | 9,820 | 3.9 |
| 9 | Lipase M^{a} | Mucor javanicus | 20.0 | 8,170 | 2.3 |
| 10 | Lipase PS^{a} | Pseudomonas | 21.6 | 8,000 | 2.3 |
| | | cepacia | | | |
| 11 | Lipase Porcine | Porcine pancreas | 2.4 | ND | ND |
| | Pancreas^{b} | | | | |

^{a}Obtained from Amano Enzyme Co (Troy, VA).
^{b}Obtained from Sigma Chemical Co (St Louis, MO).
^{c}Determined by titration.
ND = Not determined.

There was significant variation in the inulin conversion and molecular weight⁸ obtained as a function of the enzyme, but in all cases the products were water soluble. “Proleather”, an alkaline protease from Bacillus subtilis, showed the highest conversion⁹.

The number average molecular weight (Mₙ) of the polymer formed was also influenced by the source of the enzyme, and this was mainly due to the extent of reaction conversion, an expected finding given the mechanism of AA-BB polycondensation reactions¹⁰. The relatively high polydispersities are expected with such a mechanism given the large size of the inulin “monomers” in the polymerization reaction. The polymer obtained using Proleather consisted of ca. 3-4 inulin molecules linked through adipate moieties, yet remained water soluble indicating that it was not heavily crosslinked.

Poly(Inul-DVA) synthesized by Proleather was further analyzed by NMR (¹H, ¹³C NMR, and 2-dimensional ¹H-¹³H COSY and ¹H-¹³C HMQC NMR; see Supporting Information). The calculation¹¹ of DSₜₒₜₐₗ (total degree of substitution, defined as the number of DVA molecules incorporated into inulin through single or double ester bonds per 100 inulin fructofuranoside residues) and DS_{vinyl} (defined as the number of DVA incorporated to inulin by single ester bonds, and hence retaining a vinyl ester moiety, per 100 inulin fructofuranoside residues) yielded 45.8 and 8.6% (the initial molar ratio of DVA to inulin fructofuranoside residues was 0.5), respectively, which means that most of the DVA is incorporated into the inulin through double ester bonds. Hence, adipate esters were incorporated as inter- or intramolecular crosslinks on the inulin structure.

The structure of poly(Inul-DVA)¹² revealed one predominant positional isomer in the fructofuranoside residue at the 6-position and two minor isomers at the 3 and 4 positions (24.3:11.0:10.5, at the 6, 4, and 3 positions, respectively), showing the enzyme's preference for primary hydroxyl groups¹³. Furthermore, the ¹H-¹H COSY NMR experiment indicated that the reacted fructose residues are mono-substituted since no cross-peaks were shared by the three positional isomers. Hence, the intramolecular crosslinks were between different fructose residues on the same inulin chain¹⁴.

Encouraged by these results, we proceeded to study the effect of DVA concentration on DSₜₒₜₐₗ, DS_{vinyl}, and Mₙ of Poly(Inul-DVA) (Table 11).

TABLE 11
DSₜₒₜₐₗ, DS_{vinyl}, Mₙ and M_{w}/Mₙ of poly(Inul-DVA) as a function of
initial concentration of DVA added to the reaction^{a}.

| | Theoretical DS^{b} | Obtained | Obtained | Efficiency^{d} | | |
|---|---|---|---|---|---|---|
| Entry | (%) | DSₜₒₜₐₗ^{c} (%) | DS_{vinyl}^{c} (%) | (%) | Mₙ | M_{w}/Mₙ |
| 1 | 10 | 8.5 | 1.7 | 85.0 | 6,690 | 2.6 |
| 2 | 20 | 17.5 | 2.1 | 87.5 | 8,760 | 3.1 |
| 3 | 30 | 25.0 | 4.1 | 83.3 | 11,360 | 3.3 |
| 4 | 40 | 39.1 | 7.7 | 97.8 | 14,610 | 3.5 |
| 5 | 50 | 45.8 | 8.6 | 91.6 | >14,610^{e} | — |

^{a}Reactions were performed in 30 mL of anhydrous DMF containing 17 mM inulin and a calculated amount of DVA. The reaction mixtures were shaken at 250 rpm and 50° C., for 140 h, after which were purified as before⁷ (isolated yields: 44-69%).
^{b}Calculated from the initial molar ratio of DVA to inulin fructofuranoside residues.
^{c}Degree of substitution of the products (determined by ¹H NMR).
^{d}Calculated as the ratio of the obtained DSₜₒₜₐₗ to the theoretical DS.
^{e}Higher than the exclusion limit of the GPC column, circumventing any precise determination of Mₙ.

In all cases, water-soluble derivatized inulin polymers were obtained with different DSₜₒₜₐₗ, depending on the concentration of the acyl donor, and with a coupling reaction efficiency>83%. Furthermore, the ratio of the DS_{adipate} to DS_{vinyl} is relatively constant as a function of the molar ratio of DVA to inulin employed. This indicates that the reactions of diester formation and monoester formation proceed independently. Finally, increasing the DVA concentration resulted in poly(Inul-DVA) with higher Mₙ, such that at 40% theoretical DS, 3-4 inulin monomers are crosslinked together.

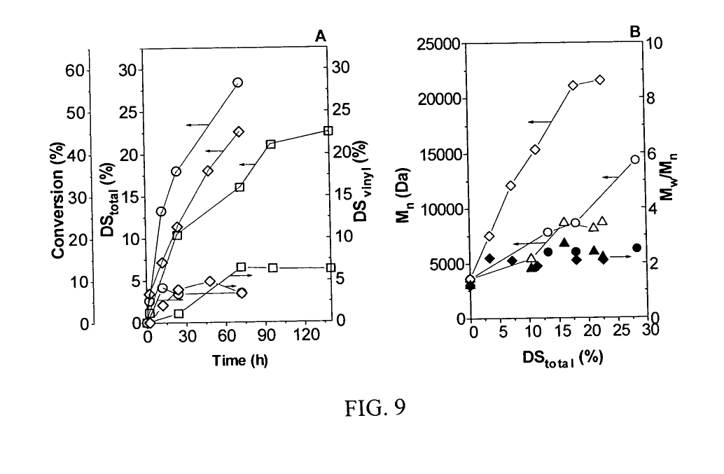

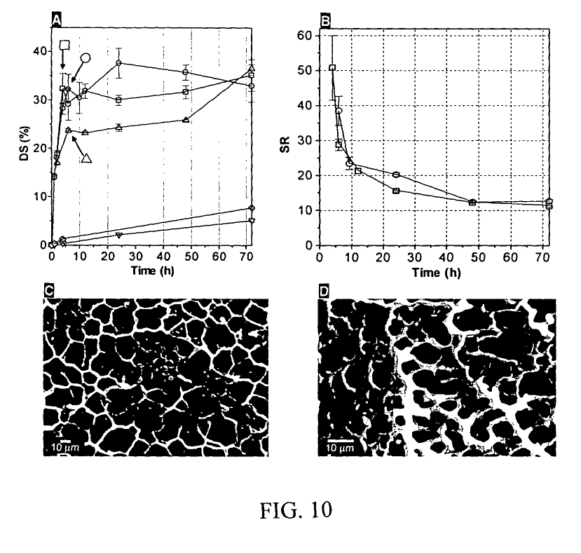

The time-course reactions of inulin with DVA, at 50° C., with different enzyme concentrations is shown in FIG. 9A. Taking into account the DSₜₒₜₐₗ, the initial incorporation of DVA into inulin molecules (reaction times<2 h) increases with increased enzyme concentration; 1.2, 2.6 and 3.5% for 10, 20, and 40 mg/mL Proleather, respectively. However, the rate of adipate incorporation into the inulin structure changes significantly at later times. Furthermore, the observed reactivity at an enzyme concentration of 40 mg/mL was lower than with 20 mg/mL. This unusual behavior may be explained by the presence of a competing reaction that results in the hydrolysis of DVA. Such a competing reaction has been observed in other polycondensation reactions performed in organic media, where traces of water associated with the enzyme promotes the hydrolysis of the highly activated divinyl esters such as DVA¹⁵. The water content of the freeze-dried Proleather was 5.6% (w/w)¹⁶; hence, sufficient amounts of water are present in the reaction mixture, and this water content would be expected to increase as the enzyme concentration is increased, thereby resulting in lower yields of polycondensation product. Finally, the incorporation of DVA molecules by a single ester moiety (DS_{vinyl}) followed almost the same trend for the different concentrations of enzyme (FIG. 9A).

The variation of Mₙ and the polydispersity of poly(Inul-DVA) versus DSₜₒₜₐₗ (FIG. 9B) was studied for the same set of experiments described in FIG. 9A. As expected, the Mₙ of poly(Inul-DVA) increased with DSₜₒₜₐₗ. Interestingly, there is a strong dependence of enzyme concentration on Mₙ; larger polymers are formed in the presence of higher enzyme concentrations. The reason for this enzyme concentration dependence is not clear.

The acylation of inulin with DVA could be conducted chemically, and this provides us an opportunity to compare directly the enzymatic and chemical approaches. To that end, we followed a chemical synthesis procedure¹⁷. The incorporation efficiency of DVA in the inulin backbone by the chemical approach (53.4%) was similar to the results achieved for the enzymatic reaction (56.7%, Proleather concentration of 20 mg/mL, reaction time of 72 h); however, poly(Inul-DVA) obtained chemically had an Mₙ of 9,580 Da (M_{w}/Mₙ=2.1), ca. 50% lower than that generated enzymatically. Thus, the enzymatic transformations yield higher molecular weight polymers than are achieved chemically. It is possible that the high degree of regioselectivity achieved enzymatically favors the formation of higher molecular weight inulin-based polymers, and our continuing work on this subject is underway.

In summary, we have demonstrated the enzyme-catalyzed polycondensation of a low molecular weight polysaccharide. To our knowledge, this is the first report dealing with enzyme-catalyzed polycondensation reactions using a polysaccharide as a monomer. These polymers may have commercial significance as polymeric drug carriers¹⁸, and carriers for magnetic resonance imaging contrast agents such as Gd¹¹¹ chelates¹⁹, and as hydrogels^{20,21}.

Supporting Information Available: Plot of Mₙ as a function of reaction conversion for the different enzymes and NMR spectra of poly(Inul-DVA). This material is available free of charge via the Internet at http://pubs.acs.org.

### REFERENCES AND NOTES FOR EXAMPLE 5

(1) Akkara, J. A.; Ayyagari, M. S. R.; Bruno, F. F. TIBTECH 1999,17, 67. Kaplan, D. L.; Dordick, J. S.; Gross, R. A., Swift, G. Enzymes in Polymer Synthesis; Gross, R. A., Kaplan, D. L., Swift, G., Eds.; ACS 684, 1996, pp. 1-15.

(2) Dordick, J. S.; Marletta, M. A.; Klibanov, A. M. Biotechnol Bioeng 1987, 30, 31.

(3) Kline, B. J.; Lele, S. S.; Lenart, P. J.; Beckman, E. J.; Russell, A. J. Biotechnol Bioeng 2000, 67, 424. Bisht, K. S.; Henderson, L. A.; Gross, R. A.; Kaplan, D. L.; Swift, G. Macromolecules 1997, 30, 2705.

(4) Al-Azemi, T. F.; Bisht, K. S. Macromolecules 1999, 32, 6536. Bisht, K. S.; Svirkin, Y. Y.; Henderson, L. A.; Gross, R. A.; Kaplan, D. L.; Swift, G. Macromolecules 1997, 30, 7735.

(5) Patil, D. R.; Rethwisch, D. G.; Dordick, J. S. Biotechnol Bioeng 1991, 37, 639. Park, O.-J.; Kim, D.-Y.; Dordick, J. S. Biotechnol Bioeng 2000, 70, 208.

(6) Roberfroid, M. B.; Van Loo, J. A. E.; Gibson, G. R. J Nutr 1998, 128, 11.

(7) The enzymes were “pH-adjusted” prior to use in the presence of 20 mM phosphate buffer at pH 8.0 (Proleather, Protease S, and subtilisin Carlsberg) or at pH 7.5 (Proteases A, N, P and Lipases A, AY, M, PS and Porcine Pancreas) following the procedure by Klibanov (Klibanov, A. M. CHEMTECH 1986, 16, 354). After flash-freezing in liquid nitrogen, the samples were lyophilized on a Labconco freeze drier (Labconco Corp., Kansas City, Mo.) for 48 h. Enzymes were screened for their reactivity on inulin by adding 300 mg of lyophilized enzyme powder (130 mg for subtilisin) to 15 mL of anhydrous DMF containing 17 mM inulin (Mₙ=3,620 Da, M_{w}/Mₙ=1.2, obtained from Fluka Chemie AG, Buchs, Switzerland) and 200 mM DVA (TCI, Portland, Oreg.). The reaction mixtures were shaken (250 rpm) at 50° C. in a temperature-controlled New Brunswick Scientific C24 orbital shaker (Edison, N.J.) for 72 h. The reactions were terminated by removing the enzyme (all enzymes were insoluble in DMF) by centrifugation at 4,000 rpm for 10 min. The supernatants were precipitated in a 4-fold excess of acetone and the precipitates were subsequently dissolved in Milli-Q water and dialyzed using a regenerated cellulose dialysis tube with a 1000 MWCO (Spectrum, Calif.) for 2 days, at 4° C., against water. Afterwards, the aqueous solutions of Inulin polyesters [poly(Inul-DVA)] were lyophilized for 48 h. The conversion was determined by back titration with 0.1 N HCl using phenolphthalein as indicator.

(8) Gel permeation chromatography (GPC) was performed with a Shimadzu LC-10AT (Columbia, Md.) equipped with a Waters 410 refractive index detector (Milford, MA). The eluent was DMF at a flow rate of 0.5 mL/min. Waters 500 and 100 Å Ultrastyragel (7.5×300 mm), and Styragel HR 5E (4.6×300 mm) were installed in series to achieve effective separation of polymers. Calibration was made with polystyrene standards of narrow polydispersity in the molecular weight range from 762 to 44,000 Da. The GPC chromatograms were obtained from samples dissolved in DMF over a concentration range of 2.1-2.4% (w/v).

(9) Two controls were performed: in the absence of enzyme, <7% conversion was obtained, while the use of thermally deactivated Proleather (i.e., boiled for 5 h followed by lyophilization) in place of the active enzyme (50° C., 24 h), conversions of ca. 5% were obtained. These results indicate that the polytransesterification reaction proceeded through enzymatic catalysis.

(10) A plot of Mₙ as a function of reaction conversion for the different enzymes studied is given in the supplemental information. The high linearity is strongly indicative of an AA-BB polycondensation reaction catalyzed by the different enzymes.

(11) Based on the ¹H NMR assignments, the DSₜₒₜₐₗ was calculated from: DSₜₒₜₐₗ=(7*z/4*y)*100 and DS_{vinyl} from: DS_{vinyl}=(7*w/y)*100, where w is the integral of the vinyl proton at δ 7.15 ppm, z is the average integral of the protons from adipate group in the range of δ 2.45-1.63 ppm and y is the integral of all inulin protons between δ 5.38-5.05 ppm and δ 4.50-3.38 ppm (see supporting information).

(12) Poly(Inul-DVA): ¹H-NMR results (δ, D₂O, ppm): δ 7.15 (dd, 1H, Hₓ), 5.38 (m, 2H, H_{1g} and H_{3f}), 5.16 (m, 2H, H_{4f} and H_{1g}), 4.94 (dd, 1H, H_{b}), 4.69 (dd, 1H, Hₐ), 4.43 (d, 1H, H_{3f-4f}), 4.23 (m, 3H, H_{6f} and H_{4f-3f}), 4.20 (d,1H, H_{3f}), 4.04 (t, 1H, H_{4f}), 3.90-3.50 (m, 5H, H_{5f}, H_{6f} and H_{1f}), 2.45 (s, 4H, adipate), 1.63 (s, 4H, adipate). Poly(Inul-DVA). ¹³C-NMR results: (δ, D₂O, ppm): δ 177.4-174.1 (C═O), 142.7 (HC═CH₂), 104.7 (C_{2f} and C_{2f-3f}), 100.7 (HC═CH₂), 94.3 (C_{1g}), 82.6 (C_{5f}), 81.3 (C_{5f-4f}), 80.2 (C_{3f}), 79.9 (C_{5f-6f}), 78.8 (C_{4f}), 78.5 (C_{3f}), 76.8 (C_{3f-4f}), 76.6 (C_{4f}), 75.8 (C_{4f}), 74.3 (C_{4f-3}f), 66.5 (C_{6f}), 63.6 (C_{6f}), 62.4 (C_{1f}), 34.8 and 34.6 (—CH₂—CH₂—CH₂—CH₂—, adipate), 25.2 and 24.9 (—CH₂—CH₂—CH₂—CH₂—, adipate). Inulin ¹³C NMR results: (δ, D₂O, ppm): δ 104.8 (C_{2f}), 82.6 (C_{5f}), 78.6 (C_{3f}), 75.8 (C_{4f}), 63.7 (C_{6f}), 62.4 (C_{1f}). The shifts observed from that of inulin are a downfield shift in C_{6f}, C_{4f} and C_{3f}. This indicates the acylation of C6f, C_{4f} and C_{3f} (denoted as C_{6f}, C_{4f} and C_{3f}) according to Yoshimoto et al. (Yoshimoto, K.; Itatani, Y.; Tsuda, Y. Chem Pharm Bull 1980, 28, 2065).

(13) Patil, D. R.; Dordick, J. S.; Rethwisch, D. G. Macromolecules 1991, 24, 3462. Riva, S.; Nonini, M.; Ottolina, G.; Danieli, B. Carbohydr Res 1998, 314, 259. Rich, J. O.; Bedell, B. A.; Dordick, J. S. Biotechnol Bioeng 1995, 45, 426.

(14) It was not possible to determine the intramolecular crosslinks content by NMR spectroscopy.

(15) Chaudhary, A. K.; Beckman, E. J.; Russell, A. J. Biotechnol Bioeng 1997, 55, 227. Brazwell, E. M.; Filos, D. Y.; Morrow, C. J. J Polym Sci Part A. Polym Chem 1995, 33, 89.

(16) As determined with a Metler LJ16 moisture analyzer (Mettler-Toledo AG, Switzerland).

(17) Following a procedure by Dijk-Wolthuis (van Dijk-Wolthuis, W. N. E.; Franssen, O.; Talsma, H.; Steenbergen, M. J.; Kettene-van den Bosch, J. J.; Hennink, W. E. Macromolecules 1995, 28, 6317), the reaction was performed in 15 mL of anhydrous DMF containing 17 mM Inulin and 200 mM DVA and initiated by addition of 200 mg 4-DMAP as catalyst. The mixture was shaken (250 rpm) at 50° C. for 72 h and then stopped by adding an equimolar concentration of concentrated HCl to neutralize the 4-DMAP. Afterwards, the reaction mixture was precipitated and washed with acetone. The precipitate was dissolved in Milli-Q water and dialyzed for 10 days at 4° C. against the same solvent. Finally, the solution was lyophilized yielding 0.129 g (yield: 9.0%, DSₜₒₜₐₗ of 26.7% and DS_{vinyl} of 3.0%) of product.

(18) Vermeersch, J.; Schacht, E. Makromol Chem 1986, 187, 125.

(19) Corsi, D. M.; Elst, L. V.; Muller, R. N.; van Bekkum, H.; Peters, J. A. Chem Eur J 2001, 7, 64.

(20) Vervoort, L.; van den Mooter, G.; Augustijns, P.; Busson, R.; Toppet, S.; Kinget, R. Pharm Res 1997, 14, 1730.

(21) We have used the free vinyl moieties that are present on the enzymatically-derivatized inulin as “monomers” for free radical polymerization. Two aqueous solutions of Poly(Inul-DVA) presenting DS_{vinyl} of 8.6% and 18.7% gel after ca. 10 min. The swelling ratio of these hydrogels in 0.01 M citrate-phosphate buffer pH 7.0 (at 25° C., for 5 days) was 34.71 and 10.83 for Poly(Inul-DVA) DS_{vinyl} 8.6% and 18.7%, respectively. Furthermore, under this pH, inulin hydrogels undergo partial ester hydrolysis as confirmed by FT-IR. Therefore, these inulin hydrogels are attractive networks for designing drug delivery systems or matrix for tissue engineering.

Modifications and variations of the invention will be obvious to those skilled in the art from the foregoing detailed description of the invention. Such modifications and variations are intended to come within the scope of the appended claims.

All patents, patent application publications and articles cited herein are incorporated by reference in their entirety.

We claim:

1. A method for making a polysaccharide derivative comprising reacting an acyl donor and a polysaccharide to form an acyl ester of the polysaccharide in a reaction medium comprising an organic solvent in the presence of a hydrolytic enzyme.

2. The method of claim 1 wherein the hydrolytic enzyme is a lipase or a protease.

3. The method of claim 1 wherein the hydrolytic protease is a bacterial protease.

4. The method of claim 1 wherein the hydrolytic protease is an alkaline protease.

5. The method of claim 1 wherein the hydrolytic enzyme is subtilisin or Proleather.

6. The method of claim 1 wherein the hydrolytic protease is a lyophilized protease.

7. The method of claim 1 wherein the reaction medium solubilizes the polysaccharide.

8. The method of claim 1 wherein the organic solvent solubilizes the polysaccharide and the acyl donor.

9. The method of claim 1 wherein the solubility of polysaccharide in the organic solvent is at least about 1 g of polysaccharide per liter of solvent at 20° C.

10. The method of claim 1 wherein the solubility of the polysaccharide in the organic solvent is at least about 10 g polysaccharide per liter of solvent at 20° C.

11. The method of claim 1 wherein the organic solvent is selected from the group consisting of pyridine, dimethylformamide, morpholine, N-methylpyrrolidone and dimethylsulfoxide.

12. The method of claim 1 wherein the reaction medium contains less than about 5% by volume water.

13. The method of claim 1 wherein the reaction medium contains less than about 1% by volume water.

14. The method of claim 1 wherein the reaction medium contains less than about 0.25% by volume water.

15. The method of claim 1 wherein the reaction medium is anhydrous.

16. The method of claim 1 wherein the polysaccharide is inulin or dextran.

17. The method of claim 1 wherein the acyl donor comprises an acyl group and at least one enzymatically-cleaved group.

18. The method of claim 17 wherein the enzymatically-cleaved group is a vinyloxy group.

19. The method of claim 17 wherein the acyl donor is a polymerizable moiety.

20. The method of claim 1 wherein the acyl donor is vinyl acrylate or methyl ester.

21. The method of claim 1 wherein the acyl donor reacts with two molecules of polysaccharide.

22. The method of claim 1 wherein the acyl donor is divinyl adipate.

23. The method of claim 1 wherein the polysaccharide has a molecular weight of at least about 700 Da.

25. A method for making a polymer comprising reacting a polymerizable acyl donor and a polysaccharide in a reaction medium comprising an organic solvent in the presence of a hydrolytic enzyme thereby making a polysaccharide monomer and polymerizing the monomer, thereby making a polymer.

26. The method of claim 24 wherein the polysaccharide is inulin and the polymer is a hydrogel.

27. The method of claim 25 wherein the inulin polymer polymerizing step is a free radical polymerization.

28. The method of claim 25 wherein the free radical polymerization is initiated by an initiator selected from the group consisting of potassium persulfate, hydrogen peroxide, azobisisobutyronitrile, benzoyl peroxide and tert-butyl peroxide.

29. The method of claim 25 wherein the polymerizing step is conducted in a reaction medium comprising an organic solvent.

30. The method of claim 25 further comprising the step of removing the enzyme from the reaction medium.

31. The method of claim 25 wherein the polymerization is a dimerization.

32. The method of claim 25 wherein the polymerizable acyl donor comprises two terminally located vinyl groups.

33. The method of claim 24 wherein the polysaccharide is characterized by a molecular weight of at least about 700 Da.

34. The method of claim 24 wherein the enzymatic esterification reaction is regiospecific.

35. The method of claim 24 wherein the Degree of Substitution is at least about 10%.

36. The method of claim 24 wherein the Swelling Ratio at Equilibrium is at least about 2.

37. The method of claim 24 wherein the average mesh size is between about 10 and 100 Å.

38. A polysaccharide derivative made by the method of claim 1.

39. A polysaccharide polymer made by the method of claim 24.

40. A cross-linked inulin characterized by a Degree of Substitution of at least about 10%, a Swelling Ratio at Equilibrium of at least about 2 and an average mesh size between about 10 and 100 Å.

41. The cross-linked inulin of claim 39 wherein inulin is cross-linked by a diester.

42. The cross-linked inulin of claim 39 wherein inulin is cross-linked with a dimerized vinyl acrylate.

43. A pharmaceutical composition comprising an active agent and a cross-linked inulin characterized by a Degree of Substitution of at least about 10%, a Swelling Ratio at Equilibrium of at least about 2 and an average mesh size between about 10 and 100 Å.

44. The pharmaceutical composition of claim 42 wherein the active agent is dispersed within the cross-linked inulin.

45. The pharmaceutical composition of claim 43 wherein the active agent is absorbed into the cross-linked inulin.

46. A method of delivering an active agent to a patient comprising administering to the patient a pharmaceutical composition comprising an active agent and a cross-linked inulin characterized by a Degree of Substitution of at least about 10%, a Swelling Ratio at Equilibrium of at least about 2 and an average mesh size between about 10 and 100 Å.

47. The method of claim 45 wherein the pharmaceutical composition is administered orally.

48. The method of claim 46 wherein the active agent is absorbed in the intestine.

49. The method of claim 47 wherein the active agent treats inflammatory bowel disorder or Crohn's disease.

50. A method of conducting an enzymatic reaction in anhydrous DMSO comprising contacting one or more enzymatic substrates solubilized in DMSO with an alkaline protease under reaction conditions thereby conducting an enzymatic reaction.

51. The method of claim 49 wherein the alkaline protease is Proleather.

52. The method of claim 49 wherein the enzymatic substrates include a polysaccharide and an acyl donor.

53. The method of claim 51 further comprising recovering an acylated polysaccharide from the reaction medium.

54. The method of claim 51 wherein the polysaccharide is selected from the group consisting of inulin and dextran.
